(19) United States
(12) Patent Application Publication
(10) Pub. No.: US 2010/0160323 A1
(43) Pub. Date: Jun. 24, 2010

(54) NOVEL PIPERAZINE DERIVATIVES AS INHIBITORS OF STEAROYL-CoA DESATURASE

(76) Inventors: Alexander Bischoff, Smithtown, NY (US); Hosahalli Subramanya, Karnataka (IN); Kumar Sundaresan, Karnataka (IN); Srinivasa Raju Sammeta, Andhra Pradesh (IN); Anil Kumar Vaka, Andhra Pradesh (IN)

Correspondence Address: Forest Laboratories, Inc.;Attn: Charles S. Ryan, 48 Mall Drive, Commack, NY, 11725, US

(21) Appl. No.: 12/644,162

(22) Filed: Dec. 22, 2009

Related U.S. Application Data
(60) Provisional application No. 61/140,288, filed on Dec. 23, 2008.

(51) Int. Cl. A61K 31/496; C07D 401/12; C07D 413/12; C07D 403/12; C07D 401/14; C07D 405/12; C07D 241/04; C07D 487/08; C07D 471/04; C07D 413/14; A61K 31/495; A61K 31/4155; A61K 31/5377; A61P 3/04; A61P 3/10

(52) U.S. Cl. 514/235.8; 544/360; 544/367; 544/371; 544/364; 544/366; 544/379; 544/387; 548/453; 544/362; 544/121; 514/253.01; 514/254.04; 514/254.05; 514/253.09; 514/252.13; 514/255.01; 514/406; 514/253.04

(57) ABSTRACT

The present invention relates to piperazine derivatives that act as inhibitors of stearoyl-CoA desaturase. The invention also relates to methods of preparing the compounds, compositions containing the compounds, and to methods of treatment using the compounds.

### FIELD OF THE INVENTION

The present invention relates to piperazine derivatives that act as inhibitors of stearoyl-CoA desaturase. The invention also relates to methods of preparing the compounds, compositions containing the compounds, and to methods of treatment using the compounds.

### BACKGROUND OF THE INVENTION

Metabolic syndrome has become one of the leading health problems in the world. As a component of metabolic syndrome, obesity also has causal roles in other components of the syndrome, including insulin resistance, dyslipidemia, and cardiovascular diseases. Effective treatments for metabolic syndrome in general and obesity in particular have been lacking. Effective therapies for the treatment of obesity, a key element of metabolic syndrome, are urgently needed.

A number of mammalian stearoyl-coenzyme A desaturase (SCD) genes have been cloned. For example, two genes have been cloned from rat (SCD1, SCD2) and four SCD genes have been isolated from mouse (SCD1, 2, 3, and 4). While the basic biochemical role of SCD has been known in rats and mice since the 1970's (see, e.g., Jeffcoat, R. et al., Elsevier Science, Vol. 4, pp. 85-112, 1984; de Antueno, R J, Lipids, Vol. 28, No. 4, pp. 285-290, 1993), it has only recently been directly implicated in human disease processes.

A single SCD gene, stearoyl-coenzyme A desaturase-1 (SCD1) has been characterized in humans. SCD1 is described, for example, in International Publication No. WO 01/62954. A second human SCD isoform has recently been identified, and because it bears little sequence homology to alternate mouse or rat isoforms it has been named human SCD5 or hSCD5 (See, e.g., International Publication No. WO 02/26944).

SCD-1 catalyzes conversion of saturated fatty acids, stearoyl-CoA and palmitoyl-CoA, to monounsaturated fatty acids, oleoyl-CoA and pamitoleoyl-CoA, respectively. These fatty acids are components of membrane phospholipids, triglycerides, and cholesterol esters. Changes in SCD activity ultimately change membrane fluidity, lipoprotein metabolism, and adiposity. SCD-1 inhibition can lead to decreased adiposity and thus be a potential therapy for metabolic syndrome.

Since obesity is becoming increasingly prevalent worldwide, much effort is being devoted to understanding its pathogenesis and treatment. In recent years, several candidate genes have been proposed as therapeutic targets. However, stearoyl-CoA desaturase 1 is of special significance, because it is the major gene target of leptin—a central mediator of energy homeostasis. There is evidence that SCD1 deficiency activates metabolic pathways that promote b-oxidation and decrease lipogenesis in liver and skeletal muscles. One mechanism is via increased activation of AMP-activated protein kinase. SCD1 mutation results also in global changes in expression of genes involved in lipid metabolism. SCD1 deficient mice have increased energy expenditure, reduced body adiposity, and are resistant to diet-induced obesity.

Thus, SCD1 inhibition represents a new and important target for the treatment of various disorders such as obesity and related metabolic disorders. Accordingly, there is a need in the art for new derivatives that act as inhibitors of stearoyl-CoA desaturase, such as SCD 1.

### SUMMARY OF THE INVENTION

The present invention relates to piperazine derivatives that act as inhibitors of stearoyl-CoA desaturase. The invention also relates to methods of preparing the compounds, compositions containing the compounds, and to methods of treatment using the compounds.

### DETAILED DESCRIPTION OF THE INVENTION

In one aspect, the present invention includes compounds with chemical structure:

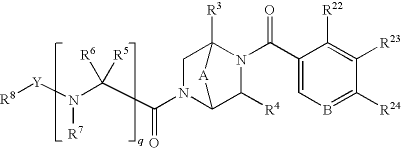

wherein

R²² is hydrogen, halogenated alkyl or halogen;

B is N or CR²⁵ where R²⁵ is hydrogen or halogen;

Y is —C(O)— or —CH₂—;

A is absent or is —CH₂—;

R³ and R⁴ are each, independently, hydrogen or alkyl;

R⁵ to Ware each, independently, hydrogen or alkyl;

R⁸ is aryl or heteroaryl;

q is 0 or 1;

provided, however, that when q is 0, then y is —C(O)—, A is absent, and B is CR²⁵;

provided, however, that when R²² is hydrogen or halogen, then B is CR²⁵, and R²⁵ is halogen;

provided, however, that when R²² is hydrogen, then R²³ to R²⁵ are each independently halogen, and B is CR²⁵;

provided, however, that when R²² is halogenated alkyl, then R²³ to R²⁴ are both hydrogen and B is CR²⁵ or N;

wherein, when present, any aryl, heteroaryl, or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkylhio, arylthio, alkylsulfinyl, alkylsulfonyl, arylsulfinyl, arylsulfonyl, heteroarylsulfinyl, heteroarylsulfonyl alkoxycarbonyl, aryloxycarbonyl, heteroaryloxycarbonyl, and combinations thereof;

and pharmaceutically acceptable salts or solvates or N-oxides thereof, or solvates of pharmaceutically acceptable salts thereof, or pharmaceutically acceptable salts or solvates of N-oxides thereof; or prodrugs thereof.

In another aspect, compounds having formula I-III are represented in the chemical structure

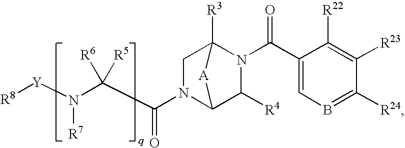

wherein the variables are as described above.

In another aspect, the present invention includes compounds of formula I:

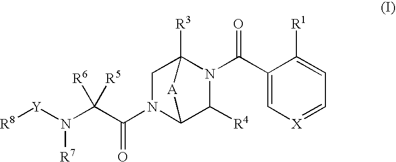

wherein

R¹ is halogenated alkyl (e.g., CF₃);

X is N or CR² where R² is hydrogen or halogen;

Y is —C(O)— or —CH₂—;

A is absent or is —CH₂—;

R³ and R⁴ are each, independently, hydrogen or alkyl;

R⁵ to Rare each, independently, hydrogen or alkyl; and

R⁸ is aryl or heteroaryl;

wherein, when present, any aryl, heteroaryl, or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkylhio, arylthio, alkylsulfinyl, alkylsulfonyl, arylsulfinyl, arylsulfonyl, heteroarylsulfinyl, heteroarylsulfonyl alkoxycarbonyl, aryloxycarbonyl, heteroaryloxycarbonyl, and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof, or prodrugs thereof.

In certain embodiments, R¹ is CF₃.

In additional embodiments, X is N, CH or C-halogen (e.g., C—F, C—Cl).

In certain embodiments, R¹ is CF₃ and X is CF. In other embodiments, R¹ is CF₃ and X is N.

In further embodiments, R³ and R⁴ are hydrogen. In other embodiments, R³ and R⁴ are alkyl (e.g., methyl).

In additional embodiments, Z is absent. In other embodiments, Z is —CH₂—. In additional embodiments, when Z is —CH₂—, R³ and R⁴ are hydrogen.

In further embodiments, R⁵ and R⁶ and hydrogen. In other embodiments, R⁷ is hydrogen.

In certain embodiments R⁸ is aryl or heteoraryl. For example, R⁸ is optionally substituted phenyl, optionally substituted naphthalenyl, optionally substituted isoxazolyl, optionally substituted triazolyl, optionally substituted pyrazolyl, optionally substituted pyridinyl or optionally substituted imidazopyridinyl.

For further example, R⁸ is naphthalenyl (e.g., naphthalene-2-yl), (pyridinyl)phenyl (e.g., 4-pyridin-3-yl-phenyl), (hydroxyphenyl)isoxazolyl (e.g., (2-hydroxyphenyl)isoxazol-3-yl), (phenyl)triazolyl (e.g., 1-phenyl-1H-[1,2,3]triazol-4-yl), (thienyl)phenyl (e.g., 4-thiophen-3-yl-phenyl), (fluorophenyl)triazolyl (e.g., 2-fluorophenyl-1H-[1,2,3]triazol-4-yl), ((methyl)isoxazolyl)triazolyl (e.g., (5-methyl-isoxazol-3-yl)-1H-[1,2,3]triazol-4-yl), (hydroxyphenyl)triazolyl (e.g., 1-(2-hydroxyphenyl)-1H-[1,2,3]triazole-4-yl), (fluorophenyl)pyrazolyl (e.g., 5-(3-fluorophenyl)-1H-pyrazole-3-yl, 5-(4-fluorophenyl)-1H-pyrazole-3-yl), phenylpyrazolyl (e.g., 4-phenyl-pyrazol-1-yl), (fluorophenyl)isoxazolyl (e.g., 5-(4-fluorophenyl)-isoxazol-3-yl), (pyrrolidinyl)pyridinyl (e.g., 6-(pyrrolidin-1-yl)pyridin-3-yl), (pyridinyl)triazolyl (e.g., 1-pyridin-3-yl-1H-[1,2,3]triazol-4-yl), (hydroxyphenyl)pyrazolyl (e.g., 5-(3-hydroxyphenyl)-1H-pyrazol-3-yl, 5-(4-hydroxyphenyl)-1H-pyrazol-3-yl), (oxadiazolyl)phenyl (e.g., 3-[1,3,4]oxadiazol-2-yl)phenyl), (aminophenyl)isoxazolyl (e.g., 5-(2-aminophenyl)-isoxazol-3-yl), (phenyl)pyrazolyl (e.g., 5-phenyl-1H-pyrazol-3-yl), (tetrazolyl)phenyl (e.g., 4-(1H-tetrazol-5-yl)phenyl), phenyl(triazolyl) (e.g., 1-phenyl-1H-[1,2,3]triazol-4-yl), imidazo[1,2-a]pyridinyl (e.g., imidazo[1,2-a]pyridine-2-yl) or (pyridinyl)pyrazolyl (e.g., 5-(pyridine-3-yl)-1H-pyrazol-3-yl)).

In another embodiment, the present invention includes compounds of formula I wherein

R¹ is halogenated alkyl (e.g., CF₃);

X is N or CR² where R² is hydrogen or halogen;

Y is —C(O)— or —CH₂—;

A is absent or is —CH₂—;

R³ and R⁴ are each, independently, hydrogen or alkyl;

R⁵ to R⁷ are hydrogen;

R⁸ is aryl or heteroaryl;

wherein, when present, any aryl, heteroaryl, or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkylhio, arylthio, alkylsulfinyl, alkylsulfonyl, arylsulfinyl, arylsulfonyl, heteroarylsulfinyl, heteroarylsulfonyl alkoxycarbonyl, aryloxycarbonyl, heteroaryloxycarbonyl, and combinations thereof.

In a further embodiment, the present invention includes compounds of formula I wherein

R¹ is halogenated alkyl (e.g., CF₃);

X is N or CR² where R² is hydrogen or halogen;

Y is —C(O)— or —CH₂—;

A is absent or is —CH₂—;

R³ and R⁴ are each, independently, hydrogen or alkyl;

R⁵ to Ware hydrogen;

R⁸ is aryl or heteroaryl;

wherein, when present, any aryl, heteroaryl, or heterocycle group may optionally be substituted by halogen, amino, nitro, alkyl, aryl, heteroaryl, heterocycle, and combinations thereof.

In further embodiments, formula I is represented by subformula Ia:

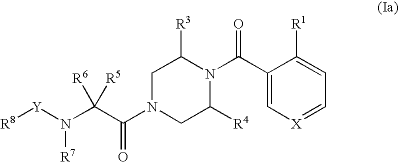

In yet further embodiments, formula I is represented by subformula Ib:

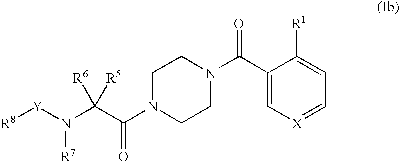

In certain embodiments, the compound of formula I is selected from:
N-{2-Oxo-2-[4-(2-trifluoromethyl-benzoyl)-piperazin-1-yl]-ethyl}-4-pyridin-3-yl-benzamide,
5-(2-Hydroxy-phenyl)-isoxazole-3-carboxylic acid {2-[4-(5-fluoro-2-trifluoromethyl-benzoyl)-piperazin-1-yl]-2-oxo-ethyl}-amide,
5-(2-Amino-phenyl)-isoxazole-3-carboxylic acid {2-oxo-2-[4-(2-trifluoromethyl-benzoyl)-piperazin-1-yl]-ethyl}-amide,
3-[1,3,4]Oxadiazol-2-yl-N-{2-oxo-2-[4-(2-trifluoromethyl-benzoyl)-piperazin-1-yl]-ethyl}-benzamide,
Naphthalene-2-carboxylic acid {2-[4-(5-fluoro-2-trifluoromethyl-benzoyl)-piperazin-1-yl]-2-oxo-ethyl}-amide,
5-Phenyl-1H-pyrazole-3-carboxylic acid {2-[3,5-dimethyl-4-(2-trifluoromethyl-benzoyl)-piperazin-1-yl]-2-oxo-ethyl}-amide,
1-Phenyl-1H-[1,2,3]triazole-4-carboxylic acid {2-oxo-2-[4-(4-trifluoromethyl-pyridine-3-carbonyl)-piperazin-1-yl]-ethyl}-amide,
5-Phenyl-1H-pyrazole-3-carboxylic acid {2-[5-(5-fluoro-2-trifluoromethyl-benzoyl)-2,5-diaza-bicyclo[2.2.1]hept-2-yl]-2-oxo-ethyl}-amide,
1-[4-(5-Fluoro-2-trifluoromethyl-benzoyl)-piperazin-1-yl]-2-(4-thiophen-3-yl-benzylamino)-ethanone,
N-{2-[4-(5-Fluoro-2-trifluoromethyl-benzoyl)-piperazin-1-yl]-2-oxo-ethyl}-4-(1H-tetrazol-5-yl)-benzamide,
1-(2-Fluoro-phenyl)-1H-[1,2,3]triazole-4-carboxylic acid {2-[4-(5-fluoro-2-trifluoromethyl-benzoyl)-piperazin-1-yl]-2-oxo-ethyl}-amide,
1-(5-Methyl-isoxazol-3-yl)-1H-[1,2,3]triazole-4-carboxylic acid {2-[4-(5-fluoro-2-trifluoromethyl-benzoyl)-piperazin-1-yl]-2-oxo-ethyl}-amide,
1-[4-(5-Fluoro-2-trifluoromethyl-benzoyl)-piperazin-1-yl]-2-[(1-phenyl-1H-[1,2,3]triazol-4-ylmethyl)-amino]-ethanone,
1-(2-Hydroxy-phenyl)-1H-[1,2,3]triazole-4-carboxylic acid {2-[4-(5-fluoro-2-trifluoromethyl-benzoyl)-piperazin-1-yl]-2-oxo-ethyl}-amide,
5-(3-Fluoro-phenyl)-1H-pyrazole-3-carboxylic acid {2-[4-(5-fluoro-2-trifluoromethyl-benzoyl)-piperazin-1-yl]-2-oxo-ethyl}-amide,
5-(4-Fluoro-phenyl)-1H-pyrazole-3-carboxylic acid {2-[4-(5-fluoro-2-trifluoromethyl-benzoyl)-piperazin-1-yl]-2-oxo-ethyl}-amide,
4-Phenyl-pyrazole-1-carboxylic acid {2-oxo-2-[4-(4-trifluoromethyl-pyridine-3-carbonyl)-piperazin-1-yl]-ethyl}-amide,
5-(4-Fluoro-phenyl)-isoxazole-3-carboxylic acid {2-[4-(5-fluoro-2-trifluoromethyl-benzoyl)-piperazin-1-yl]-2-oxo-ethyl}-amide,
N-{2-[4-(5-Fluoro-2-trifluoromethyl-benzoyl)-piperazin-1-yl]-2-oxo-ethyl}-6-pyrrolidin-1-yl-nicotinamide,
1-Pyridin-3-yl-1H-[1,2,3]triazole-4-carboxylic acid {2-[4-(5-fluoro-2-trifluoromethyl-benzoyl)-piperazin-1-yl]-2-oxo-ethyl}-amide,
Imidazo[1,2-a]pyridine-2-carboxylic acid {2-[4-(5-fluoro-2-trifluoromethyl-benzoyl)-piperazin-1-yl]-2-oxo-ethyl}-amide,
5-(3-Hydroxy-phenyl)-1H-pyrazole-3-carboxylic acid {2-[4-(5-fluoro-2-trifluoromethyl-benzoyl)-piperazin-1-yl]-2-oxo-ethyl}-amide,
5-(4-Hydroxy-phenyl)-1H-pyrazole-3-carboxylic acid {2-[4-(5-fluoro-2-trifluoromethyl-benzoyl)-piperazin-1-yl]-2-oxo-ethyl}-amide,
5-Pyridin-3-yl-1H-pyrazole-3-carboxylic acid {2-[4-(5-fluoro-2-trifluoromethyl-benzoyl)-piperazin-1-yl]-2-oxo-ethyl}-amide,
9H-Carbazole-3-carboxylic acid {2-[4-(5-fluoro-2-trifluoromethyl-benzoyl)-piperazin-1-yl]-2-oxo-ethyl}-amide,
5-(3-Hydroxy-phenyl)-isoxazole-3-carboxylic acid {2-[4-(5-fluoro-2-trifluoromethyl-benzoyl)-piperazin-1-yl]-2-oxo-ethyl}-amide,
5-(4-Hydroxy-phenyl)-isoxazole-3-carboxylic acid {2-[4-(5-fluoro-2-trifluoromethyl-benzoyl)-piperazin-1-yl]-2-oxo-ethyl}-amide,
5-(3-Fluoro-phenyl)-isoxazole-3-carboxylic acid {2-[4-(5-fluoro-2-trifluoromethyl-benzoyl)-piperazin-1-yl]-2-oxo-ethyl}-amide,
5-Pyridin-2-yl-1H-pyrazole-3-carboxylic acid {2-[4-(5-fluoro-2-trifluoromethyl-benzoyl)-piperazin-1-yl]-2-oxo-ethyl}-amide,
1-(3-Fluoro-phenyl)-1H-[1,2,3]triazole-4-carboxylic acid {2-[4-(5-fluoro-2-trifluoromethyl-benzoyl)-piperazin-1-yl]-2-oxo-ethyl}-amide,
1-(3-Fluoro-phenyl)-1H-[1,2,3]triazole-4-carboxylic acid {2-[4-(2-chloro-5-fluoro-benzoyl)-piperazin-1-yl]-2-oxo-ethyl}-amide,
4-(2-Fluoro-phenyl)-pyrazole-1-carboxylic acid {2-oxo-2-[4-(4-trifluoromethyl-pyridine-3-carbonyl)-piperazin-1-yl]ethyl}-amide,
1-m-Tolyl-1H-[1,2,3]triazole-4-carboxylic acid {2-[4-(5-fluoro-2-trifluoromethyl-benzoyl)-piperazin-1-yl]-2-oxo-ethyl}-amide,
1-o-Tolyl-1H-[1,2,3]triazole-4-carboxylic acid {2-[4-(5-fluoro-2-trifluoromethyl-benzoyl)-piperazin-1-yl]-2-oxo-ethyl}-amide,
4-(5-Fluoro-2-trifluoromethyl-benzoyl)-1-{2-[(5-phenyl-1H-pyrazole-3-carbonyl)-amino]-acetyl}-piperazine-2-carboxylic acid, and
1-(5-Fluoro-2-trifluoromethyl-benzoyl)-4-{2-[(5-phenyl-1H-pyrazole-3-carbonyl)-amino]-acetyl}-piperazine-2-carboxylic acid,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In additional embodiments, the compound of formula I is selected from:
1-[4-(5-Fluoro-2-trifluoromethyl-benzoyl)-piperazin-1-yl]-2-(4-thiophen-3-yl-benzylamino)-ethanone hydrochloride, and
1-[4-(5-Fluoro-2-trifluoromethyl-benzoyl)-piperazin-1-yl]-2-[(1-phenyl-1H-[1,2,3]triazol-4-ylmethyl)-amino]-ethanone hydrochloride,

wherein a compound listed above can also be in the form of a solvate (such as a hydrate),

wherein a compound listed above can also be in the form of an N-oxide,

wherein a compound listed above (in a solvate or N-oxide thereof) can also be in the form of a polymorph, and

wherein if the compound exhibits chirality it can be in the form of a mixture of enantiomers such as a racemate or a mixture of diastereomers, or can be in the form of a single enantiomer or a single diastereomer.

According to another aspect, the present invention includes compounds of formula II:

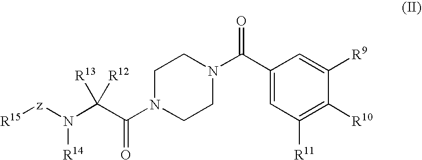

wherein

Z is —C(O)— or —CH₂—;

R⁹, R¹⁰ and R¹¹ are each, independently, halogen (e.g., F);

R¹², R¹³ and R¹⁴ are each independently hydrogen or alkyl; and

R¹⁵ is aryl or heteroaryl;

wherein, when present, any aryl, heteroaryl, or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkylhio, arylthio, alkylsulfinyl, alkylsulfonyl, arylsulfinyl, arylsulfonyl, heteroarylsulfinyl, heteroarylsulfonyl alkoxycarbonyl, aryloxycarbonyl, heteroaryloxycarbonyl and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof.

In certain embodiments, Z is —C(O)—. In additional embodiments, Z is —CH₂—.

In further embodiments, R⁹, R¹⁰ and R¹¹ are each F.

In yet further embodiments, R¹², R¹³ and R¹⁴ are each hydrogen.

In certain embodiments R¹⁵ is aryl or heteoraryl. For example, R¹⁵ is optionally substituted pyrazolyl, optionally substituted phenyl or optionally substituted triazolyl.

For further example, R¹⁵ is (hydroxyphenyl)pyrazolyl (e.g., 5-(2-hydroxyphenyl)-1H-pyrazol-3-yl), biphenyl (e.g., 4-biphenyl), (pyridinyl)triazolyl (e.g., 1-pyridin-3-yl-1H-[1,2,3]triazol-4-yl, 1-pyridin-2-yl-1H-[1,2,3]triazol-4-yl) or (cyclopentyl)triazolyl (e.g., 1-cyclopentyl-1H-[1,2,3]triazol-4-yl).

In another embodiment, the present invention includes compounds of formula II wherein

Z is —C(O)— or —CH₂—;

R⁹, R¹⁰ and R¹¹ are each, independently, halogen (e.g., F);

R¹², R¹³ and R¹⁴ are hydrogen; and

R¹⁵ is aryl or heteroaryl;

wherein, when present, any aryl, heteroaryl, or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkylhio, arylthio, alkylsulfinyl, alkylsulfonyl, arylsulfinyl, arylsulfonyl, heteroarylsulfinyl, heteroarylsulfonyl alkoxycarbonyl, aryloxycarbonyl, heteroaryloxycarbonyl and combinations thereof.

In a further embodiment, the present invention includes compounds of formula II wherein

Z is —C(O)— or —CH₂—;

R⁹, R¹⁰ and R¹¹ are each, independently, halogen (e.g., F); R¹², R¹³ and R¹⁴ are hydrogen; and

R¹⁵ is aryl or heteroaryl;

wherein, when present, any aryl, heteroaryl, or heterocycle group may optionally be substituted by hydroxy, cycloalkyl, aryl, heteroaryl, and combinations thereof.

In certain embodiments, the compound of formula II is selected from:
5-(2-Hydroxy-phenyl)-1H-pyrazole-3-carboxylic acid {2-oxo-2-[4-(3,4,5-trifluoro-benzoyl)-piperazin-1-yl]-ethyl}-amide,
2-[(Biphenyl-4-ylmethyl)-amino]-1-[4-(3,4,5-trifluoro-benzoyl)-piperazin-1-yl]-ethanone,
1-Pyridin-3-yl-1H-[1,2,3]triazole-4-carboxylic acid {2-oxo-2-[4-(3,4,5-trifluoro-benzoyl)-piperazin-1-yl]-ethyl}-amide,
1-Pyridin-2-yl-1H-[1,2,3]triazole-4-carboxylic acid {2-oxo-2-[4-(3,4,5-trifluoro-benzoyl)-piperazin-1-yl]ethyl}-amide,
1-Cyclopentyl-1H-[1,2,3]triazole-4-carboxylic acid {2-oxo-2-[4-(3,4,5-trifluoro-benzoyl)-piperazin-1-yl]-ethyl}-amide,
1-(2-Fluoro-phenyl)-1H-[1,2,3]triazole-4-carboxylic acid {2-[4-(2-chloro-5-fluoro-benzoyl)-piperazin-1-yl]-2-oxo-ethyl}-amide, and
1-m-Tolyl-1H-[1,2,3]triazole-4-carboxylic acid {2-oxo-2-[4-(3,4,5-trifluoro-benzoyl)-piperazin-1-yl]-ethyl}-amide,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In additional embodiments, the compound of formula II is selected from:
2-[(Biphenyl-4-ylmethyl)-amino]-1-[4-(3,4,5-trifluoro-benzoyl)-piperazin-1-yl]-ethanone hydrochloride,

wherein the compound listed above can also be in the form of a solvate (such as a hydrate),

wherein the compound listed above can also be in the form of an N-oxide,

wherein the compound listed above (in a solvate or N-oxide thereof) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In a further aspect, the present invention includes compounds of formula III:

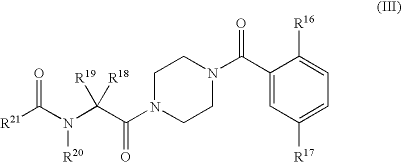

wherein

R¹⁶ and R¹⁷ are each, independently, halogen (e.g., F, Br, Cl);

R¹⁸, R¹⁹ and R²⁰ are each, independently, hydrogen or alkyl; and

R²¹ is aryl or heteroaryl;

wherein, when present, any aryl, heteroaryl, or heterocycle group may optionally be substituted by halogen, hydroxy, cyano, nitro, amino, alkylamino, dialkylamino, arylamino, oxo, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkylhio, arylthio, alkylsulfinyl, alkylsulfonyl, arylsulfinyl, arylsulfonyl, heteroarylsulfinyl, heteroarylsulfonyl alkoxycarbonyl, aryloxycarbonyl, heteroaryloxycarbonyl, and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof.

In certain embodiments, R¹⁶ is Cl and R¹⁷ is F.

In additional embodiments, R¹⁸, R¹⁹ and R²⁰ are hydrogen.

In certain embodiments R²¹ is aryl or heteoraryl. For example, R²¹ is optionally substituted imidazolyl, optionally substituted phenyl or optionally substituted triazolyl.

For further example, R²¹ is (pyridinyl)triazolyl (e.g., 1-pyridin-3-yl-1H-[1,2,3]triazol-4-yl, 1-pyridin-2-yl-1H-[1,2,3]triazol-4-yl), (phenyl)imidazolyl (e.g., 1-phenyl-1H-imidazol-4-yl), (cyclopropyl)triazolyl (e.g., 1-cyclopropyl-1H-[1,2,3]triazol-4-yl), (morpholinyl)triazolyl (e.g., 1-morpholin-4-yl-1H-[1,2,3]triazol-4-yl), (oxopyrrolidinyl)phenyl (e.g., 4-(2-oxo-pyrrolidin-1-yl)phenyl or (cyclopentyl)triazolyl (e.g., 1-cyclopentyl-1H-[1,2,3]triazol-4-yl).

In certain embodiments, the compound of formula III is selected from:
1-Pyridin-3-yl-1H-[1,2,3]triazole-4-carboxylic acid {2-[4-(2-chloro-5-fluoro-benzoyl)-piperazin-1-yl]-2-oxo-ethyl}-amide,
1-Pyridin-2-yl-1H-[1,2,3]triazole-4-carboxylic acid {2-[4-(2-chloro-5-fluoro-benzoyl)-piperazin-1-yl]-2-oxo-ethyl}-amide,
1-Phenyl-1H-imidazole-4-carboxylic acid {2-[4-(2-chloro-5-fluoro-benzoyl)-piperazin-1-yl]-2-oxo-ethyl}-amide,
1-Cyclopropyl-1H-[1,2,3]triazole-4-carboxylic acid {2-[4-(2-chloro-5-fluoro-benzoyl)-piperazin-1-yl]-2-oxo-ethyl}-amide,
1-Morpholin-4-yl-1H-[1,2,3]triazole-4-carboxylic acid {2-[4-(2-chloro-5-fluoro-benzoyl)-piperazin-1-yl]-2-oxo-ethyl}-amide,
N-2-[4-(2-Chloro-5-fluoro-benzoyl)-piperazin-1-yl]-2-oxo-ethyl-4-(2-oxo-pyrrolidin-1-yl)-benzamide,
1-Cyclopentyl-1H-[1,2,3]triazole-4-carboxylic acid {2-[4-(2-chloro-5-fluoro-benzoyl)-piperazin-1-yl]-2-oxo-ethyl}-amide,
1-(2-Fluoro-phenyl)-1H-[1,2,3]triazole-4-carboxylic acid {2-[4-(2-chloro-5-fluoro-benzoyl)-piperazin-1-yl]-2-oxo-ethyl}-amide,
1-m-Tolyl-1H-[1,2,3]triazole-4-carboxylic acid {2-[4-(2-chloro-5-fluoro-benzoyl)-piperazin-1-yl]-2-oxo-ethyl}-amide,
1-(2-Cyano-phenyl)-1H-[1,2,3]triazole-4-carboxylic acid {2-[4-(2-chloro-5-fluoro-benzoyl)-piperazin-1-yl]-2-oxo-ethyl}-amide,
5-(4-Fluoro-phenyl)-isoxazole-3-carboxylic acid {2-[4-(2-chloro-5-fluoro-benzoyl)-piperazin-1-yl]-2-oxo-ethyl}-amide, and
6-Pyrazol-1-yl-imidazo[1,2-a]pyridine-2-carboxylic acid {2-[4-(2-chloro-5-fluoro-benzoyl)-piperazin-1-yl]-2-oxo-ethyl}-amide,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In certain embodiments, the compounds of the present invention are chosen from:
2-{2-Oxo-2-[4-(2-trifluoromethyl-benzoyl)-piperazin-1-yl]-acetylamino}-benzoic acid methyl ester,
2-Oxo-N-(6-phenyl-pyridin-3-yl)-2-[4-(2-trifluoromethyl-benzoyl)-piperazin-1-yl]-acetamide,
N-Biphenyl-4-yl-2-[4-(5-fluoro-2-trifluoromethyl-benzoyl)-piperazin-1-yl]-2-oxo-acetamide,
[4-(5-Fluoro-2-trifluoromethyl-benzoyl)-piperazin-1-yl]-oxo-acetic acid ethyl ester,
3-{2-[4-(5-Fluoro-2-trifluoromethyl-benzoyl)-piperazin-1-yl]-2-oxo-acetylamino}-N-methyl-benzamide,
2-[4-(5-Fluoro-2-trifluoromethyl-benzoyl)-piperazin-1-yl]-2-oxo-N-(4-phenoxy-phenyl)-acetamide,
N-Biphenyl-4-yl-2-[4-(2-bromo-benzoyl)-piperazin-1-yl]-2-oxo-acetamide, and
N-Biphenyl-4-yl-2-oxo-2-[4-(3,4,5-trifluoro-benzoyl)-piperazin-1-yl]-acetamide,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

As used herein the term “halogen” means F, Cl, Br, and I.

The term “alkyl” means a substituted or unsubstituted saturated hydrocarbon radical which may be straight-chain or branched-chain and may comprise about 1 to about 20 carbon atoms, for instance 1 to 12 carbon atoms, such as 1 to 8 carbon atoms, e.g., 1 to 4 carbon atoms. Suitable alkyl groups include, but are not limited to, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to, 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, methylhexyl, dimethylpentyl, ethylpentyl, ethylmethylbutyl, dimethylbutyl, and the like.

Substituted alkyl groups are alkyl groups as described above which are substituted in one or more positions by, e.g., halogen, hydroxyl, amino, carboxy, alkylamino, dialkylamino, aryl, heteroaryl, alkoxy, nitro and cyano, and combinations thereof.

The term “halogenated alkyl” means a saturated hydrocarbon radical which may be straight-chain or branched-chain and may comprise about 1 to about 20 carbon atoms, for instance 1 to 12 carbon atoms, such as 1 to 8 carbon atoms, e.g., 1 to 4 carbon atoms, that is substituted by one or more halogens, such as, but not limited to, —CF₃, CF₂CF₃, CHF₂, CH₂F, and the like. The use of the term “halogenated alkyl” should not be construed to mean that a “substituted alkyl” group may not be substituted by one or more halogens.

The term “alkenyl” means a substituted or unsubstituted hydrocarbon radical which may be straight-chain or branched-chain, which contains one or more carbon-carbon double bonds, and which may comprise about 1 to about 20 carbon atoms, such as 1 to 12 carbon atoms, for instance 1 to 6 carbon atoms. Suitable alkenyl groups include ethenyl, propenyl, butenyl, etc.

Substituted alkenyl groups are alkenyl groups as described above which are substituted in one or more positions by, e.g., halogen, hydroxyl, amino, carboxy, alkylamino, dialkylamino, aryl, heteroaryl, alkoxy, nitro and cyano, and combinations thereof.

The term “alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

The term “alkynyl” means a substituted or unsubstituted aliphatic hydrocarbon radical which may be straight-chain or branched-chain and which contains one or more carbon-carbon triple bonds. Preferably the alkynyl group contains 2 to 15 carbon atoms, such as 2 to 12 carbon atoms, e.g., 2 to 8 carbon atoms. Suitable alkynyl groups include ethynyl, propynyl, butynyl, etc.

Substituted alkynyl groups are alkynyl groups as described above which are substituted in one or more positions by, e.g., halogen, hydroxyl, amino, carboxy, alkylamino, dialkylamino, aryl, heteroaryl, alkoxy, nitro and cyano, and combinations thereof.

The term “amino” means —NH₂.

The term “alkylamino” means —NH(alkyl), wherein alkyl is as described above.

The term “dialkylamino” means —N(alkyl)₂, wherein alkyl is as described above.

The term “aryl” means a substituted or unsubstituted aromatic monocyclic or bicyclic ring system comprising about 5 to about 14 carbon atoms, e.g., about 6 to about 10 carbon atoms. Suitable aryl groups include, but are not limited to, phenyl, naphthyl, anthracenyl.

Substituted aryl groups include the above-described aryl groups which are substituted one or more times by, for example, but not limited to, halogen, hydroxyl, amino, carboxy, alkylamino, dialkylamino, aryl, heteroaryl, alkoxy, nitro and cyano, and combinations thereof.

The term “arylamino” means —NH(aryl), wherein aryl is as described above.

The term “diarylamino” means —N(aryl)₂, wherein aryl is as described above.

The term “amido” means —CONH₂.

The term “alkylamido” means a —CONH(alkyl) group, wherein alkyl is as described above.

The term “dialkylamido” means a —CON(alkyl)₂ group, wherein alkyl is as described above.

The term “aminoalkyl” means a -(alkylene)-amino, -(alkylene)-alkylamino or -(alkylene)-dialkylamino group, wherein the various groups are as described above.

The term “arylalkyl” refers to an -(alkylene)-aryl group in which the aryl and alkylene portions are in accordance with the previous descriptions. Suitable examples include, but are not limited to, benzyl, 1-phenethyl, 2-phenethyl, phenpropyl, phenbutyl, phenpentyl, and napthylmethyl.

The term “carboxyl” means —C(O)OH.

The term “cycloalkyl” means a monocyclic, bicyclic or tricyclic nonaromatic saturated hydrocarbon radical having 3 to 10 carbon atoms, such as 3 to 8 carbon atoms, for example, 3 to 6 carbon atoms. Suitable cycloalkyl groups include, but are not limited to, cyclopropyl, cyclobutyl, cyclopentyl, cyclohexyl, cycloheptyl, cyclooctyl, norbornyl, 1-decalin, adamant-1-yl, and adamant-2-yl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, bicyclo[4.2.0]octyl, and spiro[3.5]nonyl. Preferred cycloalkyl groups include cyclopropyl, cyclobutyl, cyclopentyl and cyclohexyl. The cycloalkyl group can be substituted, for example, by one or more halogens and/or alkyl groups.

The term “cycloalkylalkyl” means a -(alkylene)-cycloalkyl in which the cycloalkyl group is as previously described; e.g., cyclopropylmethyl, cyclobutylmethyl, cyclopentylethyl, or cyclohexylmethyl, and the like.

The term “heteroaryl” means a substituted or unsubstituted aromatic monocyclic or multicyclic ring system comprising 5 to 14 ring atoms, preferably about 5 to about 10 ring atoms and most preferably 5 or 6 ring atoms, wherein at least one of the ring atoms is an N, O or S atom. Suitable heteroaryl groups include, but are not limited to furyl, thienyl, pyrrolyl, pyrazolyl, imidazolyl, pyridyl, pyrimidinyl, benzimidazolyl, indazolyl, indolyl, quinolinyl, isoquinolinyl, naphthyridinyl and the like.

Substituted heteroaryl groups include the above-described heteroaryl groups which are substituted one or more times by, for example, but not limited to, halogen, hydroxyl, amino, carboxy, alkylamino, dialkylamino, aryl, heteroaryl, alkoxy, nitro and combinations thereof.

The term “heteroarylalkyl” refers to a -(alkylene)-heteroaryl group wherein the heteroaryl and alkylene portions are in accordance with the previous discussions. Suitable examples include, but are not limited to, pyridylmethyl, thiazolylmethyl, thienylmethyl, pyrimidinylmethyl, pyrazinylmethyl, and isoquinolinylmethyl, and the like.

The term “heterocycle” means a substituted or unsubstituted non-aromatic mono- or multicyclic ring system comprising 3 to 10 atoms, preferably 5 or 6, wherein at least one of the ring atoms is an N, O or S atom. Suitable heterocyle groups include, but are not limited to tetrahydrofuranyl, tetrahydrothienyl, tetrahydropyranyl, dihydropyranyl, pyrrolidinyl, piperidinyl, piperazinyl, thiomorpholinyl, morpholinyl, isoxazolinyl, and the like

Substituted heterocycle groups include the above-described heterocycle groups which are substituted one or more times by, for example, halogen, amino, alkyl, hydroxy, carboxy, and combinations thereof. Heterocycle groups may also be substituted by, e.g., aryl or heteroaryl.

The term “heterocyclealkyl” refers to a -(alkylene)-heterocycle group wherein the heterocycle and alkylene portions are in accordance with the previous discussions.

The term “aroyl” means an aryl-C(O)—, in which the aryl group is as previously described. Suitable aroyl groups include, but are not limited to, benzoyl and 1-naphthoyl.

The term “acyl” means an HC(O)—, alkyl-C(O)—, cycloalkyl-C(O)—, aryl-C(O)—, or heteroalkyl-C(O)—, in which the various groups are as previously described, e.g., acetyl, propionyl, benzoyl, pyridinylcarbonyl, and the like.

The term “alkoxy” means alkyl-O— groups in which the alkyl portion is in accordance with the previous discussion. Suitable alkoxy groups include, but are not limited to, methoxy, ethoxy, n-propoxy, iso-propoxy, n-butoxy, t-butoxy, pentoxy, hexoxy, heptoxy, octoxy, and the like. For example, the alkoxy can be methoxy or ethoxy.

The term “aryloxy” means an aryl-O— group, in which the aryl group is as previously described.

The term “heteroaryloxy” means an heteroaryl-O— group, in which the heteroaryl group is as previously described.

The term “cycloalkylalkyloxy” means a —O-(alkylene)-cycloalkyl group, in which the cycloalkyl and alkylene groups are as previously described.

The term “alkylthio” means an alkyl-S— group, in which the alkyl group is as previously described.

The term “arylthio” means an aryl-S— group, in which the aryl group is as previously described.

The term “alkylsulfinyl” means a —SOR radical where R is alkyl as defined above, e.g., methylsulfinyl, ethylsulfinyl, and the like.

The term “alkylsulfonyl” means a —SO₂R radical where R is alkyl as defined above, e.g., methylsulfonyl, ethylsulfonyl, and the like.

The term “arylsulfinyl” means a —SOR radical where R is aryl as defined above, e.g., phenylsulfinyl, and the like.

The term “arylsulfonyl” means a —SO₂R radical where R is aryl as defined above, e.g., phenylsulfonyl, and the like.

The term “heteroarylsulfinyl” means a —SOR radical where R is heteroaryl as defined above.

The term “heteroarylsulfonyl” means a —SO₂R radical where R is heteroaryl as defined above.

The term “alkoxycarbonyl” means an alkyl-O—C(O)— group, in which the alkyl group is as previously described.

The term “aryloxycarbonyl” means an aryl-O—C(O)— group, in which the aryl group is as previously described.

The term “heteroaryloxycarbonyl” means an heteroaryl-O—C(O)— group, in which the heteroaryl group is as previously described.

The term “cycloalkyloxy” means a —O-cycloalkyl group in which the cycloalkyl group is as previously described, e.g., cyclopropyloxy, cyclobutyloxy, cyclopentyloxy, cyclohexyloxy, and the like

The term “arylalkyloxy” means —O-(alkylene)-aryl group, in which the aryl and alkylene groups are as previously described.

The term “heteroarylalkyloxy” means —O-(alkylene)-heteroaryl group, in which the heteroaryl and alkylene groups are as previously described.

One of ordinary skill in the art will recognize that compounds of formulas I-III can exist in different tautomeric and geometrical isomeric forms. All of these compounds, including cis isomers, trans isomers, diastereomic mixtures, racemates, nonracemic mixtures of enantiomers, substantially pure, and pure enantiomers, are within the scope of the present invention. Substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of formulas I-III can likewise be obtained by utilizing optically active starting materials in chiral synthesis processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] (2000), 110 pp. CAN 133:68895 AN 2000:473538 CAPLUS; Kabalka, George W.; Varma, Rajender S. The synthesis of radiolabeled compounds via organometallic intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem. (1981), 64(1-2), 9-32. CODEN: JRACBN ISSN:0022-4081, CAN 95:76229 AN 1981:476229 CAPLUS.

Where applicable, the present invention also relates to useful forms of the compounds as disclosed herein, such as base free forms, and pharmaceutically acceptable salts or prodrugs of all the compounds of the present invention for which salts or prodrugs can be prepared. Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, aDIPEAtes, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride, a hydrobromide, a hydroformate, or a maleate, for example, a hydrochloride.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of formulas I-III can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of formulas I-III can exist in different solvate forms. Solvates of the compounds of the invention may also form when solvent molecules are incorporated into the crystalline lattice structure of the compound molecule during the crystallization process.

The present invention also includes prodrugs of compounds of formulas I-III. The term prodrug is intended to represent covalently bonded carriers, which are capable of releasing the active ingredient of formulas I-III when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of formulas I-III include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of formulas amides (e.g., trifluoroacetylamino, acetylamino, and the like), and the like. Prodrugs of compounds of formulas I-III are also within the scope of this invention.

The present invention also provides processes for preparing the compounds of formulas I-III. Suitable general reaction schemes are shown below.

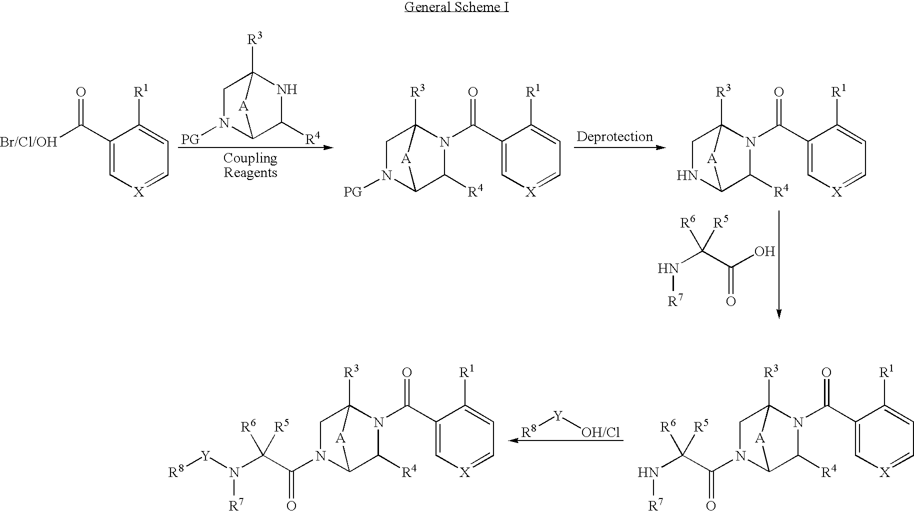

A monoprotected (PG) piperazine is reacted with an appropriately substituted aryl acid or acid halide under coupling reaction conditions (e.g., EDCI/HOBT) using a suitable solvent, such as DMF. The coupled product is then deprotected and coupled with an unsubstituted or substituted protected (for example Boc protected) glycine. Deprotection of the resulting product, followed by a coupling reaction with a substituted or unsubstituted aryl or heteroaryl acid or acid halide affords a compound of formula I.

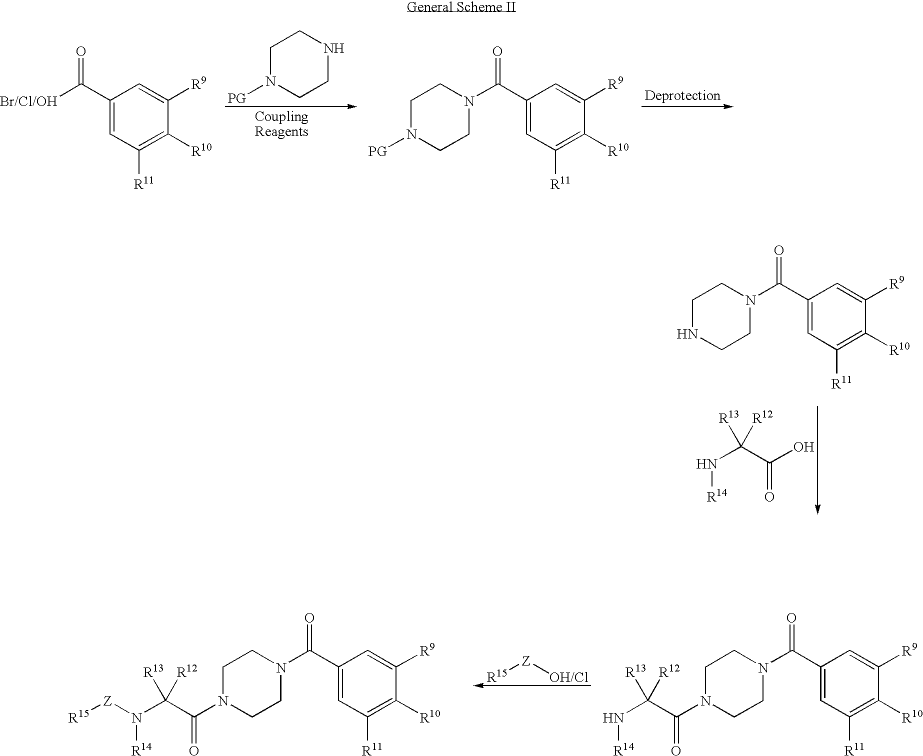

A monoprotected (PG) piperazine is reacted with an appropriately substituted aryl acid or acid halide under coupling reaction conditions (e.g., EDCI/HOBT) using a suitable solvent, such as DMF. The coupled product is then deprotected and coupled with an unsubstituted or substituted protected (for example Boc protected) glycine. Deprotection of the resulting product, followed by a coupling reaction with a substituted or unsubstituted aryl or heteroaryl acid or acid halide affords a compound of formula II.

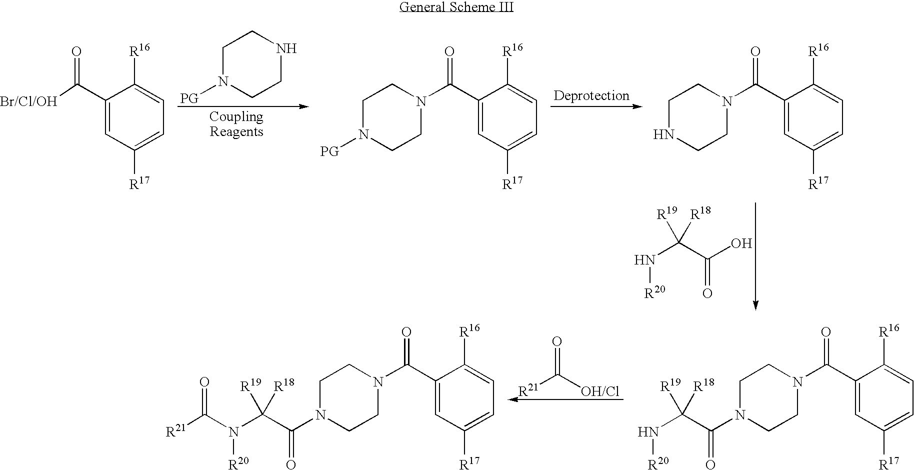

A monoprotected (PG) piperazine is reacted with an appropriately substituted aryl acid or acid halide under coupling reaction conditions (e.g., EDCI/HOBT) using a suitable solvent, such as DMF. The coupled product is then deprotected and coupled with an unsubstituted or substituted protected (for example Boc protected) glycine. Deprotection of the resulting product, followed by a coupling reaction with a substituted or unsubstituted aryl or heteroaryl acid or acid halide affords a compound of formula III.

The compounds of the invention can be administered alone or as an active ingredient of a formulation. Thus, the present invention also includes pharmaceutical compositions of compounds of formulas I-III, or combinations or mixtures thereof, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), (current edition).

Administration of the compounds of the present invention may be accomplished according to patient needs, for example, orally, nasally, parenterally (subcutaneously, intravenously, intramuscularly, intrasternally and by infusion) by inhalation, rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, the compounds of the invention can be administered by inhalation in the form of a powder (e.g., micronized) or in the form of atomized solutions or suspensions. The aerosol formulation can be placed into a pressurized acceptable propellant.

The compounds of the present invention may be useful as inhibitors of stearoyl-CoA desaturase (SCD) enzymes, for example, as inhibitors of SCD-1 enzyme. Therefore, the compounds are useful in the treatment, preventment or management of conditions mediated by stearoyl-CoA desaturase (SCD) enzymes, e.g., SCD-1 enzyme.

According to one embodiment, the present invention relates to a method of treating, preventing or managing a disease or condition mediated by stearoyl-CoA desaturase (e.g., SCD-1) by administering to a patient in need thereof a therapeutically effective amount of a compound of formula I, II or III, or a combination or mixture thereof.

According to another embodiment, the present invention relates to a method of treating, preventing or managing a condition that responds to a stearoyl-CoA desaturase (e.g., SCD-1) inhibitor by administering to a patient in need thereof a therapeutically effective amount of a compound of formula I, II or III, or a combination or mixture thereof.

According to another embodiment, the present invention relates to a method of modulating stearoyl-CoA desaturase (e.g., SCD-1) activity in a cell by administering to a patient in need thereof a therapeutically effective amount of a compound of formula I, II or III, or a combination or mixture thereof.

An SCD-mediated disease or condition includes but is not limited to a disease or condition which is, or is related to, cardiovascular disease, dyslipidemias (including but not limited to disorders of serum levels of triglycerides, hypertriglyceridemia, VLDL, HDL, LDL, fatty acid Desaturation Index (e.g. the ratio of 18:1/18:0 fatty acids, or other fatty acids), cholesterol, and total cholesterol, hypercholesterolemia, as well as cholesterol disorders (including disorders characterized by defective reverse cholesterol transport), familial combined hyperlipidemia, coronary artery disease, atherosclerosis, heart disease, cerebrovascular disease (including, but not limited to stroke, ischemic stroke and transient ischemic attack (TIA)), peripheral vascular disease, and ischemic retinopathy. In an embodiment, compounds of the invention will, in a patient, increase HDL levels and/or decrease triglyceride levels and/or decrease LDL or non-HDL-cholesterol levels.

An SCD-mediated disease or condition also includes metabolic syndrome (including but not limited to dyslipidemia, obesity and insulin resistance, hypertension, microalbuminemia, hyperuricaemia, and hypercoagulability), Syndrome X, diabetes, insulin resistance, decreased glucose tolerance, non-insulin-dependent diabetes mellitus, Type II diabetes, Type I diabetes, diabetic complications, body weight disorders (including but not limited to obesity, overweight, cachexia and anorexia), weight loss, body mass index and leptin related diseases. In an embodiment, the compounds of the present invention are useful in the treatment, prevention or management of diabetes (e.g., diabetes mellitus) In another embodiment, the compounds of the present invention are useful in the treatment, prevention or management of obesity.

As used herein, the term “metabolic syndrome” is a recognized clinical term used to describe a condition comprising combinations of Type II diabetes, impaired glucose tolerance, insulin resistance, hypertension, obesity, increased abdominal girth, hypertriglyceridemia, low HDL, hyperuricaemia, hypercoagulability and/or microalbuminemia.

An SCD-mediated disease or condition also includes fatty liver, hepatic steatosis, hepatitis, non-alcoholic hepatitis, non-alcoholic steatohepatitis (NASH), alcoholic hepatitis, acute fatty liver, fatty liver of pregnancy, drug-induced hepatitis, erythrohepatic protoporphyria, iron overload disorders, hereditary hemochromatosis, hepatic fibrosis, hepatic cirrhosis, hepatoma and conditions related thereto.

An SCD-mediated disease or condition also includes, but is not limited to, a disease or condition which is, or is related to primary hypertriglyceridemia, or hypertriglyceridemia secondary to another disorder or disease, such as hyperlipoproteinemias, familial histiocytic reticulosis, lipoprotein lipase deficiency, apolipoprotein deficiency (such as ApoCII deficiency or ApoE deficiency), and the like, or hypertriglyceridemia of unknown or unspecified etiology.

An SCD-mediated disease or condition also includes a disorder of polyunsaturated fatty acid (PUFA) disorder, or a skin disorder, including, but not limited to, eczema, acne, psoriasis, keloid scar formation or prevention, diseases related to production or secretions from mucous membranes, such as monounsaturated fatty acids, wax esters, and the like.

An SCD-mediated disease or condition also includes inflammation, sinusitis, asthma, pancreatitis, osteoarthritis, rheumatoid arthritis, cystic fibrosis, and pre-menstrual syndrome.

An SCD-mediated disease or condition also includes a disease or condition which is, or is related to cancer, neoplasia, malignancy, metastases, tumours (benign or malignant), carcinogenesis, hepatomas and the like.

An SCD-mediated disease or condition also includes a condition where increasing lean body mass or lean muscle mass is desired, such as is desirable in enhancing performance through muscle building. Myopathies and lipid myopathies such as carnitine palmitoyltransferase deficiency (CPT I or CPT II) are also included herein. Such treatments are useful in humans and in animal husbandry, including administration to bovine, porcine or avian domestic animals or any other animal to reduce triglyceride production and/or provide leaner meat products and/or healthier animals.

An SCD-mediated disease or condition also includes a disease or condition which is, or is related to, neurological diseases, psychiatric disorders, multiple sclerosis, eye diseases, and immune disorders.

An SCD-mediated disease or condition also includes a disease or condition which is, or is related to, viral diseases or infections including, but not limited, to all positive strand RNA viruses, coronaviruses, SARS virus, SARS-associated coronavirus, Togaviruses, Picornaviruses, Coxsackievirus, Yellow Fever virus, Flaviviridae, ALPHAVIRUS (TOGAVIRIDAE) including Rubella virus, Eastern equine encephalitis virus, Western equine encephalitis virus, Venezuelan equine encephalitis virus, Sindbis virus, Semliki forest virus, Chikungunya virus, O'nyong'nyong virus, Ross river virus, Mayaro virus, Alphaviruses; ASTROVIRIDAE including Astrovirus, Human Astroviruses; CALICIVIRIDAE including Vesicular exanthema of swine virus, Norwalk virus, Calicivirus, Bovine calicivirus, Pig calcivirus, Hepatitis E; CORONAVIRIDAE including Coronavirus, SARS virus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Berne virus, Breda virus; FLAVIVIRIDAE including Hepatitis C virus, West Nile virus, Yellow Fever virus, St. Louis encephalitis virus, Dengue Group, Hepatitis G virus, Japanese B encephalitis virus, Murray Valley encephalitis virus, Central European tick-borne encephalitis virus, Far Eastern tick-borne encephalitis virus, Kyasanur forest virus, Louping ill virus, Powassan virus, Omsk hemorrhagic fever virus, Kumilinge virus, Absetarov anzalova hypr virus, Ilheus virus, Rocio encephalitis virus, Langat virus, Pestivirus, Bovine viral diarrhea, Hog cholera virus, Rio Bravo Group, Tyuleniy Group, Ntaya Group, Uganda S Group, Modoc Group; PICORNAVIRIDAE including Coxsackie A virus, Rhinovirus, Hepatitis A virus, Encephalomyocarditis virus, Mengovirus, ME virus, Human poliovirus 1, Coxsackie B; POTYVIRIDAE including Potyvirus, Rymovirus, Bymovirus. Additionally it can be a disease or infection caused by or linked to Hepatitis viruses, Hepatitis B virus, Hepatitis C virus, human immunodeficiency virus (HIV) and the like. Treatable viral infections include those where the virus employs an RNA intermediate as part of the replicative cycle (hepatitis or HIV); additionally it can be a disease or infection caused by or linked to RNA negative strand viruses such as influenza and parainfluenza viruses.

In one embodiment, the compounds of the invention are useful in the treatment, prevention or management of elevated levels of lipids, cardiovascular diseases, diabetes, obesity, and metabolic syndrome. For example, the compounds of the present invention are useful in the treatment, prevention or management of obesity and diabetes (e.g., Type II diabetes, diabetes mellitus).

In the treatment, prevention or management of conditions which require stearoyl-CoA desaturase receptor modulation an appropriate dosage level will generally be about 0.001 to 100 mg per kg patient body weight per day which can be administered in single or multiple doses. For example, the dosage level will be about 0.01 to about 25 mg/kg per day, such as about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions may be provided in the form of tablets containing about 1 to about 1000 milligrams of the active ingredient, such as about 1, about 5, about 10, about 15, about 20, about 25, about 50, about 75, about 100, about 150, about 200, about 250, about 300, about 400, about 500, about 600, about 750, about 800, about 900 or about 1000 milligrams of the active ingredient. The compounds may be administered on a regimen of 1 to 4 times per day, for example, once or twice per day.

It will be understood, however, that the specific dose level and frequency of dosage for any particular patient may be varied and will depend upon a variety of factors including the activity of the specific compound employed, the metabolic stability and length of action of that compound, the age, body weight, hereditary characteristics, general health, sex and diet of the subject, as well as the mode and time of administration, rate of excretion, drug combination, and the severity of the particular condition for the subject undergoing therapy.

In some embodiments, the compounds of the present invention are administered as a mono-therapy. In other embodiments, the compounds of the present invention are administered as part of a combination therapy. For example, a compound of formula I, II or III, or a combination or mixture thereof, may be used in combination with other drugs or therapies that are used in the treatment, prevention, suppression, management or amelioration of the diseases or conditions for which compounds of formulas I-III are useful.

Such other drug(s) may be administered, by a route and in an amount commonly used therefor, contemporaneously or sequentially with a compound of formula I, II or III, or a combination or mixture thereof. When a compound of formula I, II or III, or a combination or mixture thereof is used contemporaneously with one or more other drugs, a pharmaceutical unit dosage form containing such other drugs in addition to the compound of formula I, II or III, or a combination or mixture thereof, may be employed. Accordingly, the pharmaceutical compositions of the present invention include those that also contain one or more other active ingredients, in addition to a compound of formula I, II or III, or a combination or mixture thereof.

The term “treating” means to relieve, alleviate, delay, reduce, reverse, improve, manage or prevent at least one symptom of a condition in a subject. The term “treating” may also mean to arrest, delay the onset (i.e., the period prior to clinical manifestation of a disease) and/or reduce the risk of developing or worsening a condition.

An “effective amount” means the amount of a compound of formulas I, II or III, or a combination or mixture thereof, that, when administered to a patient (e.g., a mammal) for treating a disease, is sufficient to effect such treatment for the disease to achieve the objectives of the invention. The “effective amount” will vary depending on the compound, the disease and its severity and the age, weight, etc., of the patient to be treated.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, such as up to 10%, for example, up to 5%.

### EXAMPLES

The present invention will now be further described by way of the following non-limiting examples. In applying the disclosure of these examples, it should be kept clearly in mind that other and different embodiments of the methods and synthetic schemes disclosed according to the present invention will no doubt suggest themselves to those of skill in the relevant art.

The syntheses of additional piperazine derivatives that act as stearoyl Co-A desaturase ligands are described in U.S. application Ser. No. 12,144,604, filed Jun. 23, 2008, which claims the benefit of U.S. Provisional Application No. 60,951,997, filed Jul. 26, 2007, and Indian Application No. 1280/CHE/2007, filed Jun. 21, 2007.

In the foregoing and in the following examples, all temperatures are set forth uncorrected in degrees Celsius; and, unless otherwise indicated, all parts and percentages are by weight.

The following abbreviations may be used herein: Ac (CH₃CO), BINAP (2,2′-bis(diphenylphosphino)-1,1′-binaphthyl), Bn (benzyl), DCM (dichloromethane), DMF (dimethylformamide), DIPEA (1-ethyl-3-[3-dimethylaminopropyl]carbodiimide hydrochloride), EDCI (1-ethyl-3-[3-dimethylaminopropyl]carbodiimide hydrochloride), Et (ethyl), HOBT (1-hydroxybenzotriazole), Me (methyl), TFA (trifluoroacetic acid), THF (tetrahydrofuran), EtOAc (ethyl acetate), MeOH (methanol), Pd(OAc)₂ (palladium acetate), K₂CO₃ (potassium carbonate), HCOONH₄ (ammonium formate), Pd/C (palladium on carbon), Boc (tert-butoxycarbonyl), Na₂SO₄ (sodium sulphate), NaHCO₃ (sodium bicarbonate) HCl (hydrochloric acid), HBr (hydrogen bromide), NaCl (sodium chloride), brine (saturated sodium chloride solution), CHCl₃ (chloroform), Cs₂CO₃ (caesium carbonate, cesium carbonate), NaClO₂ (sodium chlorite), NH₃SO₃ [NH₂.SO₃H] (Sulphamic acid), NaOH (sodium hydroxide), celite (diatomaceous earth), TLC (thin layer chromatography), NMR (nuclear magnetic resonance), DMSO-d₆ (deuterated dimethyl sulfoxide), CDCl₃ (deuterated chloroform), LC-MS (LC-MS liquid chromatography-mass spectrometry), HPLC (high pressure liquid chromatography or high performance liquid chromatography), DCE (1,2-dichloroethane), xantphos (4,5-bis(diphenylphosphino)-9,9-dimethylxanthene).

### Example 1

### N-{2-Oxo-2-[4-(2-trifluoromethyl-benzoyl)-piperazin-1-yl]-ethyl}-4-pyridin-3-yl-benzamide

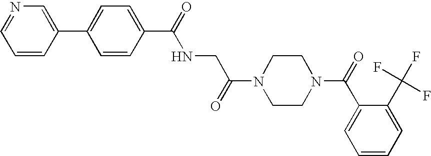

DIPEA (116.32 mg, 0.15 mL, 0.9 mmol) followed by HOBT (48 mg, 0.36 mmol) and EDCI (70 mg, 0.36 mmol) were added to a stirred solution of 4-pyridin-3-yl-benzoic acid (60 mg, 0.3 mmol) in DMF (1.5 mL) at room temperature. After 2 minutes, 2-amino-1-[4-(2-trifluoromethyl-benzoyl)-piperazin-1-yl]-ethanone hydrochloride (127 mg, 0.36 mmol) (prepared according to a procedure similar to that described in Synthesis Procedure 1, Steps 1A-1E, using HCl/MeOH or 1,4-dioxane in place of trifluoroacetic acid in the last step) was added and the resulting mixture was stirred at room temperature overnight. The reaction mixture was partitioned between cold water and ethyl acetate. The organic layer was washed with brine, dried over Na₂SO₄ and concentrated under reduced pressure to afford 29 mg (19%) of N-{2-oxo-2-[4-(2-trifluoromethyl-benzoyl)-piperazin-1-yl]-ethyl}-4-pyridin-2-yl-benzamide. LCMS Purity: 89.36%. ¹H NMR (DMSO-d₆): δ 9-8.9 (d, 1H), 8.6 (d, 1H), 8.5 (m, 1H), 8.2-8.1 (dt, 1H), 8.0 (d, 2H), 7.9-7.6 (m, 5H), 7.6-7.5 (m, 2H), 4.2 (m, 2H), 3.8-3.6 (m, 4H), 3.2-3.4 (m, 2H), 3.2-3 (m, 2H).

### Example 2

### 5-(2-Hydroxy-phenyl)-isoxazole-3-carboxylic acid {2-[4-(5-fluoro-2-trifluoromethyl-benzoyl)-piperazin-1-yl]-2-oxo-ethyl}-amide

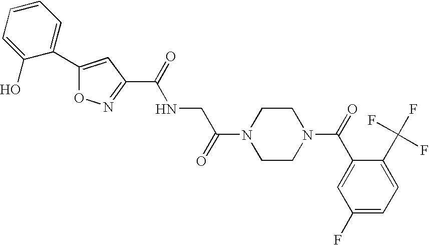

10% Pd/C (40 mg) was added to a stirred solution of 5-(2-benzyloxy-phenyl)-isoxazole-3-carboxylic acid {2-oxo-2-[4-(2-trifluoromethyl-benzoyl)-piperazin-1-yl]-ethyl}-amide (74 mg, 0.12 mmol) (prepared according to a procedure similar to that described in Synthesis Procedure 3, Steps 1-4-b, using 1-(2-hydroxyphenyl)ethanone (Aldrich, St. Louis, Mo.) as a starting material) in MeOH (30 mL) and hydrogenated for 1.5 hrs. The mixture was then filtered over celite. The residue was washed with MeOH and the filtrate was concentrated under reduced pressure. The resulting residue was washed with ethyl acetate to afford 26 mg (81%) of 5-(2-hydroxy-phenyl)-isoxazole-3-carboxylic acid {2-[4-(5-fluoro-2-trifluoromethyl-benzoyl)-piperazin-1-yl]-2-oxo-ethyl}-amide. LCMS Purity: 95.85%. ¹H NMR (DMSO-d₆): δ 10.8 (s, 1H), 8.7 (t, 1H), 7.9 (m, 1H), 7.78 (m, 1H), 7.48 (m, 2H), 7.3 (m, 1H), 6.9 (m, 3H), 4.1 (m, 2H), 3.5 (m, 4H), 3.4 (m, 2H), 3.0 (m, 2H).

### Example 3

### 5-(2-Hydroxy-phenyl)-1H-pyrazole-3-carboxylic acid {2-oxo-2-[4-(3,4,5-trifluoro-benzoyl)-piperazin-1-yl]-ethyl}-amide

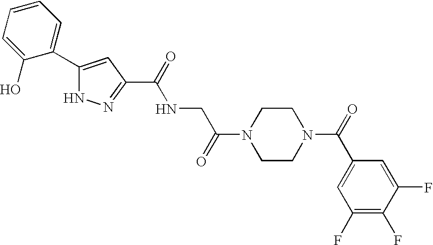

### Step 1

### Synthesis of {[5-(2-Hydroxy-phenyl)-1H-pyrazole-3-carbonyl]-amino}-acetic acid

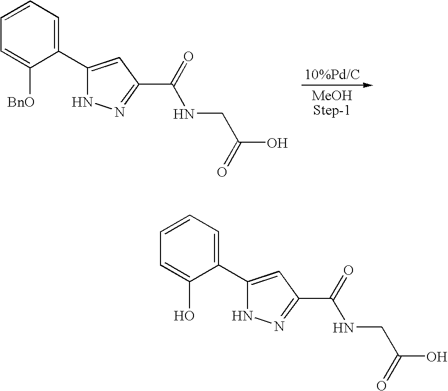

10% Pd/C (40 mg) was added to a stirred solution of {[5-(2-benzyloxy-phenyl)-1H-pyrazole-3-carbonyl]-amino}-acetic acid (74 mg, 0.12 mmol) prepared according to a procedure similar to that described in Synthesis Procedure 4, Steps 1-3 and 5-7, using O-benzylated 1-(2-hydroxyphenyl)ethanone (Aldrich, St. Louis, Mo.) as a starting material) in MeOH (30 mL) and the mixture was hydrogenated for 3 hrs. The mixture was then filtered over celite. The residue was washed with MeOH and the filtrate was concentrated under reduced pressure to afford 110 mg (99%) of {[5-(2-hydroxy-phenyl)-1H-pyrazole-3-carbonyl]-amino}-acetic acid. ¹H NMR (DMSO-d₆): δ 10.4-10.2 (bs, 1H), 8.5-8.2 (bs, 1H), 7.7-7.6 (d, 1H), 7.2-7.1 (t, 1H), 7.0 (d, 1H), 6.9 (t, 1H), 4.0-3.8 (d, 2H).

### Step 2

### Synthesis of 5-(2-Hydroxy-phenyl)-1H-pyrazole-3-carboxylic acid {2-oxo-2-[4-(3,4,5-trifluoro-benzoyl)-piperazin-1-yl]-ethyl}-amide

DIPEA (90 mg, 0.12 mL, 0.7 mmol) followed by HOBT (34 mg, 0.25 mmol) and EDCI (100 mg, 0.57 mmol) were added to a stirred solution of {[5-(2-Hydroxy-phenyl)-1H-pyrazole-3-carbonyl]-amino}-acetic acid (60 mg, 0.23 mmol) in DMF (3 mL) at room temperature. After 2 minutes of stirring, piperazin-1-yl-(3,4,5-trifluoro-phenyl)-methanone hydrochloride (61.8 mg, 0.25 mmol) (prepared according to a procedure similar to that described in Synthesis Procedure 1, using 3,4,5-trifluorobenzoic acid (Aldrich, St. Louis, Mo.) as a starting material) was added and the resulting mixture was stirred at room temperature for 5 hrs. The reaction mixture was then partitioned between cold water and ethyl acetate. The organic layer was subjected to a brine wash, dried over Na₂SO₄ and concentrated under reduced pressure to afford 53 mg (47%) of 5-(2-hydroxy-phenyl)-1H-pyrazole-3-carboxylic acid {2-oxo-2-[4-(3,4,5-trifluoro-benzoyl)-piperazin-1-yl]-ethyl}-amide. LCMS Purity: 98.98%. ¹H NMR (DMSO-d₆): δ 8.3-8.0 (bs, 1H), 7.7 (d, 1H), 7.5 (t, 2H), 7.3-7.1 (t, 2H), 7.0-6.8 (m, 2H), 4.28-4.1 (bs, 2H), 3.7-3.44 (bs, 6H).

### General Scheme A

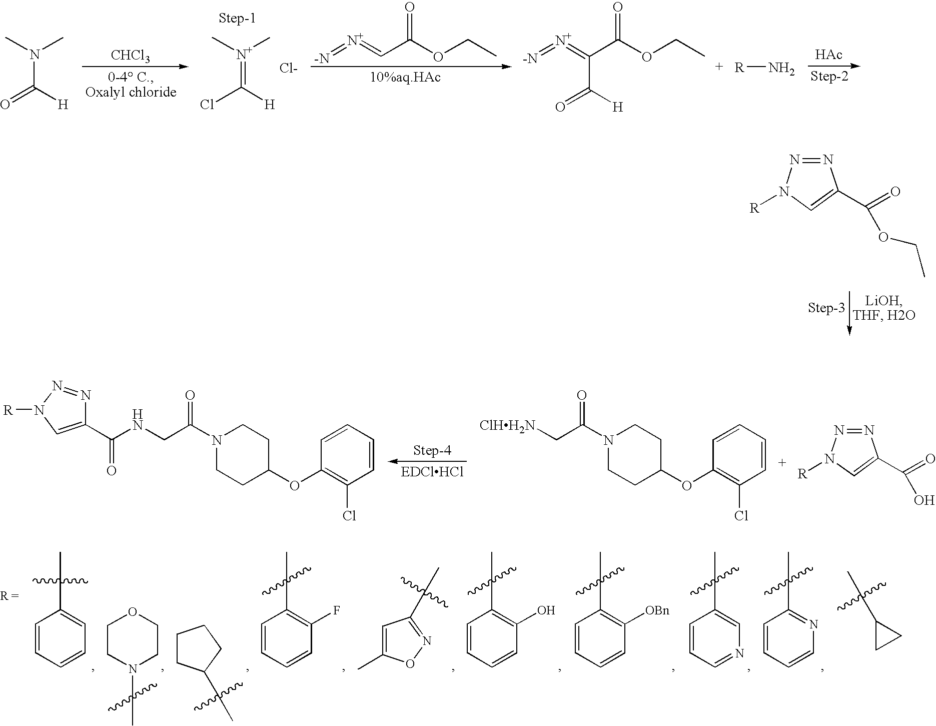

### Step 1

### Synthesis of 2-Diazo-3-oxo-propionic acid ethyl ester

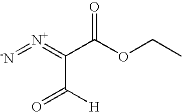

Oxalyl chloride (4.7 g, 3.1 mL, 37.0 mmol) was added to a cold (−4° C.) solution of DMF (2.25 g, 2.4 mL, 30.8 mmol) in CHCl₃ (20 mL) and the reaction was stirred at this temperature for 10 minutes, followed by heating at 40° C. for a further 10 minutes. After chilling the reaction to −10° C., diazo-acetic acid ethyl ester (3.5 g, 3.5 mL, 30.6 mmol) was added and the mixture was stirred at room temperature for 1 hr. The mixture was the concentrated and the residue was diluted with ether. The solid was collected by filtration and dissolved in 10% aq acetic acid (10 mL) then stirred for a further 1 hr. The reaction mixture was extracted with ether and the ether layer was washed with saturated sodium bicarbonate solution and brine. The organic phase was dried over Na₂SO₄ and concentrated under reduced pressure to afford 590 mg (21%) of 2-diazo-3-oxo-propionic acid ethyl ester. ¹H NMR (CDCl₃): δ 9.7 (s, 1H), 4.4 (q, 2H), 1.4 (t, 3H).

### Step 2

### Synthesis of 1-Phenyl-1H-[1,2,3]triazole-4-carboxylic acid ethyl ester

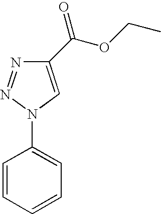

Aniline (143 mg, 1.5 mmol) was added to a stirred solution of 2-diazo-3-oxo-propionic acid ethyl ester (200 mg, 1.4 mmol) and acetic acid (0.2 mL) in EtOH (0.5 mL) and stirring was continued at room temperature overnight. The reaction mixture was then concentrated and the residue was diluted with cold water. The precipitate was collected to afford 264 mg (87%) of 1-phenyl-1H-[1,2,3]triazole-4-carboxylic acid ethyl ester. ¹H NMR (CDCl₃): δ 8.5 (s, 1H), 7.8 (d, 2H), 7.6-7.48 (m, 3H), 4.5 (q, 2H), 1.4 (t, 3H).

### Step 3

### Synthesis of 1-Phenyl-1H-[1,2,3]triazole-4-carboxylic acid

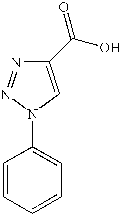

LiOH.H₂O (80 mg, 1.9 mmol) was added to a stirred solution of 1-phenyl-1H-[1,2,3]triazole-4-carboxylic acid ethyl ester (130 mg, 0.6 mmol) in THF:H₂O (1:1, 4 mL), and the resulting mixture was stirred at room temperature for 45 min. The reaction mixture was concentrated under reduced pressure. Cold water was added to the residue followed by acidification with 10% aqueous HCl. The resulting precipitate was collected afford 40 mg (35%) of 1-phenyl-1H-[1,2,3]triazole-4-carboxylic acid. ¹H NMR (DMSO-d₆): δ 13.4 (bs, 1H), 9.4 (s, 1H), 8.0 (d, 2H), 7.7 (t, 2H), 7.6 (t, 1H).

### Example 4

### Step 4

### 1-Phenyl-H-[1,2,3]triazole-4-carboxylic acid {2-oxo-2-[4-(4-trifluoromethyl-pyridine-3-carbonyl)-piperazin-1-yl]-ethyl}-amide

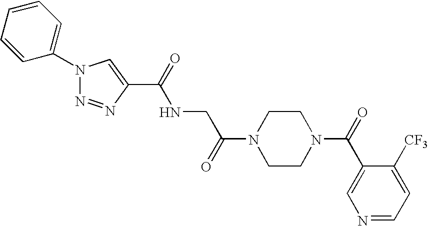

DIPEA (85 mg, 0.11 mL, 0.65 mmol) followed by HOBT (19.6 mg, 0.14 mmol) and EDCI (63.2 mg, 0.32 mmol) were added to a stirred solution of 1-phenyl-1H-[1,2,3]triazole-4-carboxylic acid (25 mg, 0.13 mmol) in DMF (3 mL) at room temperature. After 2 minutes, 2-amino-1-[4-(4-trifluoromethyl-pyridine-3-carbonyl)-piperazin-1-yl]-ethanone hydrochloride (55 mg, 0.15 mmol) (prepared according to a procedure similar to that described in Synthesis Procedure 1, using 4-(trifluoromethyl)nicotinic acid (Aldrich, St. Louis, Mo.) as a starting material) was added and the resulting mixture was stirred at room temperature overnight. Cold water was added to the reaction mixture and the mixture was extracted with ethyl acetate> The organic layer was washed with saturated brine solution, dried over sodium sulfate and concentrated under reduce pressure to afford 52 mg (80%) of 1-phenyl-1H-[1,2,3]triazole-4-carboxylic acid {2-oxo-2-[4-(4-trifluoromethyl-pyridine-3-carbonyl)-piperazin-1-yl]-ethyl}-amide. LCMS Purity: 95.96%. ¹H NMR (DMSO-d₆): δ 9.4 (s, 1H), 9.0 (d, 2H), 8.54 (s, 1H), 8.04 (d, 2H), 7.94 (s, 1H), 7.68 (t, 2H), 7.6 (t, 1H), 4.3 (d, 2H), 3.8 (s, 1H), 3.66 (s, 2H), 3.56 (d, 3H), 3.24 (d, 2H).

### Example 5

### 1-(2-Fluoro-phenyl)-1H-[1,2,3]triazole-4-carboxylic acid {2-[4-(5-fluoro-2-trifluoromethyl-benzoyl)-piperazin-1-yl]-2-oxo-ethyl}-amide

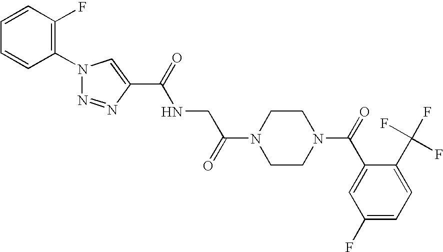

DIPEA (190 mg, 0.25 mL, 1.5 mmol) followed by HOBT (61.7 mg, 0.45 mmol) and EDCI (200 mg, 1.0 mmol) were added to a stirred solution of 1-(2-fluoro-phenyl)-1H-[1,2,3]triazole-4-carboxylic acid (prepared from 2-fluoroaniline) (86 mg, 0.41 mmol) in DMF (5 mL) at room temperature. After 2 minutes, 2-amino-1-[4-(5-fluoro-2-trifluoromethyl-benzoyl)-piperazin-1-yl]-ethanone hydrochloride (168 mg, 0.45=101) (prepared according to a procedure similar to that described in Synthesis Procedure 1, using 5-fluoro-2-(trifluoromethyl)benzoic acid (Aldrich, St. Louis, Mo.) as a starting material) was added and the resulting mixture was stirred at room temperature overnight. The addition of cold water facilitated precipitation of the product which was recrystallized from a mixture of ethyl acetate and hexane to afford 110 mg (51%) of 1-(2-fluoro-phenyl)-1H-[1,2,3]triazole-4-carboxylic acid {2-[4-(5-fluoro-2-trifluoromethyl-benzoyl)-piperazin-1-yl]-2-oxo-ethyl}-amide. LCMS Purity: 92.89%. ¹H NMR (DMSO-d₆): δ 9.1 (s, 1H), 8.6 (t, 1H), 8.0-7.8 (m, 2H), 7.6-7.4 (m, 5H), 4.3 (d, 2H), 3.8-3.4 (m, 6H), 3.24 (d, 2H).

### Example 6

### 1-(5-Methyl-isoxazol-3-yl)-1H-[1,2,3]triazole-4-carboxylic acid {2-[4-(5-fluoro-2-trifluoromethyl-benzoyl)-piperazin-1-yl]-2-oxo-ethyl}-amide

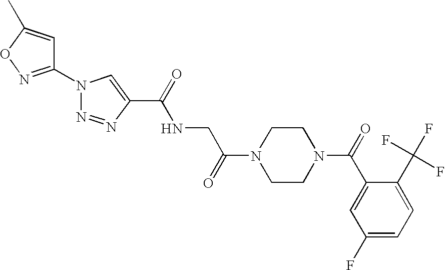

DIPEA (190 mg, 0.25 mL, 1.5 mmol) followed by HOBT (76.5 mg, 0.56 mmol) and EDCI (197 mg, 1.0 mmol) were added to a stirred solution of 1-(5-methyl-isoxazol-3-yl)-1H-[1,2,3]triazole-4-carboxylic acid (prepared by a procedure similar to that described in Steps 1-3 of General Scheme 1) (100 mg, 0.51 mmol) in DMF (3 mL) at room temperature. After 2 minutes, 2-amino-1-[4-(5-fluoro-2-trifluoromethyl-benzoyl)-piperazin-1-yl]-ethanone hydrochloride (190 mg, 045 mmol (prepared according to a procedure similar to that described in Synthesis Procedure 1, using 5-fluoro-2-(trifluoromethyl)benzoic acid (Aldrich, St. Louis, Mo.) as a starting material) was added and the resulting mixture was stirred at room temperature overnight. Cold water was added to the reaction mixture followed by extraction with ethyl acetate. The ethyl acetate layer was washed with saturated brine solution, dried over sodium sulfate and concentrated under reduce pressure. The residue was purified by column chromatography (using 60-120 silica gel and 60% EtOAc in hexane as eluent) to afford 120 mg (46%) of 1-(5-methyl-isoxazol-3-yl)-1H-[1,2,3]triazole-4-carboxylic acid {2-[4-(5-fluoro-2-trifluoromethyl-benzoyl)-piperazin-1-yl]-2-oxo-ethyl}-amide. LCMS Purity: 92.48%. ¹H NMR (DMSO-d₆): δ 9.3-9.2 (s, 1H), 8.7-8.6 (t, 1H), 8.0-7.9 (m, 1H), 7.64-7.46 (m, 2H), 7.1-7.0 (s, 1H), 4.3-4.14 (dd, 2H), 3.8-3.4 (m, 6H), 3.3-3.0 (m, 2H), 2.6-2.54 (s, 3H).

### Example 7

### 1-(2-Hydroxy-phenyl)-1H-[1,2,3]triazole-4-carboxylic acid {2-[4-(5-fluoro-2-trifluoromethyl-benzoyl)-piperazin-1-yl]-2-oxo-ethyl}-amide

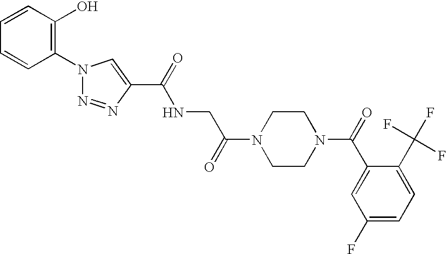

10% Pd/C (60 mg) was added to a stirred solution of 1-(2-benzyloxy-phenyl)-1H-[1,2,3]triazole-4-carboxylic acid {2-[4-(5-fluoro-2-trifluoromethyl-benzoyl)-piperazin-1-yl]-2-oxo-ethyl}-amide (200 mg, 0.12 mmol) (prepared according to a procedure similar to that described in Synthesis Procedure 2, using 2-aminophenol (Aldrich, St. Louis, Mo.) as a starting material) in MeOH (30 mL) and the mixture was hydrogenated overnight. The mixture was then filtered over celite. The residue was washed with MeOH and the filtrate was concentrated under reduced pressure. Purification of the residue by column chromatography (using 60-120 silica gel and 70% EtOAc in Hexane) afforded 80 mg (22%) of 1-(2-hydroxy-phenyl)-1H-[1,2,3]triazole-4-carboxylic acid {2-[4-(5-fluoro-2-trifluoromethyl-benzoyl)-piperazin-1-yl]-2-oxo-ethyl}-amide. LCMS Purity: 97.9%. ¹H NMR (DMSO-d₆): δ 10.7 (s, 1H), 8.9 (s, 1H), 8.5 (t, 1H), 8.0-7.9 (m, 1H), 7.7-7.5 (m, 3H), 7.4 (t, 1H), 7.2-7.1 (d, 1H), 7.1-7.0 (t, 1H), 4.3-4.1 (dd, 2H), 3.8-3.4 (m, 6H), 3.3-3.1 (m, 2H).

### Example 8

### 1-Pyridin-3-yl-1H-[1,2,3]triazole-4-carboxylic acid {2-[4-(5-fluoro-2-trifluoromethyl-benzoyl)-piperazin-1-yl]-2-oxo-ethyl}-amide

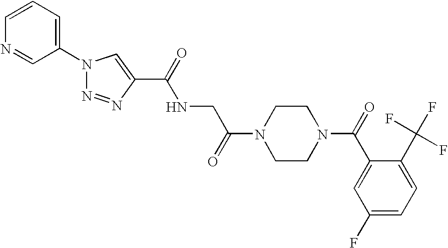

DIPEA (166 mg, 0.23 mL, 1.3 mmol) followed by HOBT (38 mg, 0.28 mmol) and EDCI (123 mg, 0.6 mmol) were added to a stirred solution of 1-pyridin-3-yl-1H-[1,2,3]triazole-4-carboxylic acid (prepared by a procedure similar to that described in Steps 1-3 of General Scheme 1) (60 mg, 0.32 mmol) in DMF (4 mL) at room temperature. After 2 minutes, 2-amino-1-[4-(5-fluoro-2-trifluoromethyl-benzoyl)-piperazin-1-yl]ethanone hydrochloride (104 mg, 0.28 mmol) (prepared according to a procedure similar to that described in Synthesis Procedure 1, using 5-fluoro-2-(trifluoromethyl)benzoic acid (Aldrich, St. Louis, Mo.) as a starting material) was added and the resulting mixture was stirred at room temperature overnight. Cold water was added and the resulting precipitate was purified by recrystallisation from a mixture of ethyl acetate and hexane to afford 87 mg (69%) of 1-pyridin-3-yl-1H-[1,2,3]triazole-4-carboxylic acid {2-[4-(5-fluoro-2-trifluoromethyl-benzoyl)-piperazin-1-yl]-2-oxo-ethyl}-amide. LCMS Purity: 96.55%. ¹H NMR (DMSO-d₆): δ 9.5 (s, 1H), 9.22 (s, 1H), 8.78 (d, 1H), 8.6 (t, 1H), 8.46 (d, 1H), 8.0 (b, 1H), 7.74 (dd, 1H), 7.64 (m, 2H), 4.3 (d, 2H), 3.84-3.4 (m, 6H), 3.24 (d, 2H).

### Example 9

### 1-Pyridin-3-yl-1H-[1,2,3]triazole-4-carboxylic acid {2-oxo-2-[4-(3,4,5-trifluoro-benzoyl)-piperazin-1-yl]-ethyl}-amide

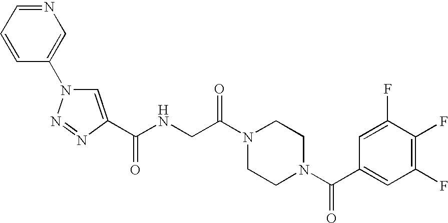

DIPEA (149 mg, 0.2 mL, 1.1 mmol) followed by HOBT (42.9 mg, 0.31 mmol) and EDCI (110 mg, 0.57 mmol) were added to a stirred solution of 1-pyridin-3-yl-1H-[1,2,3]triazole-4-carboxylic acid (55 mg, 0.29 mmol) (prepared by a procedure similar to that described in Steps 1-3 of General Scheme 1) in DMF (2 mL) at room temperature. After 2 minutes, 2-amino-1-[4-(3,4,5-trifluoro-benzoyl)-piperazin-1-yl]-ethanone hydrochloride (97.6 mg, 0.29 mmol) (prepared according to a procedure similar to that described in Synthesis Procedure 1, using 3,4,5-trifluoro benzoic acid (Aldrich, St. Louis, Mo.) as a starting material) was added and the resulting mixture was stirred at room temperature overnight. Cold water was to the reaction flask and the resulting precipitate was purified by recrystallisation from a mixture of dichloromethane and hexane to afford 63 mg (46%) of 1-pyridin-3-yl-1H-[1,2,3]triazole-4-carboxylic acid {2-oxo-2-[4-(3,4,5-trifluoro-benzoyl)-piperazin-1-yl]-ethyl}-amide. LCMS Purity: 97.28%. ¹H NMR (DMSO-d₆): δ 9.45 (s, 1H), 9.2 (d, 1H), 8.74 (dd, 1H), 8.6-8.5 (t, 1H), 8.42 (d, 1H), 7.7 (q, 1H), 7.5 (t, 2H), 4.2 (bs, 2H), 3.7-3.5 (m, 6H), 3.45-3.35 (m, 2H).

### Example 10

### 1-Pyridin-3-yl-1H-[1,2,3]triazole-4-carboxylic acid {2-[4-(2-chloro-5-fluoro-benzoyl)-piperazin-1-yl]-2-oxo-ethyl}-amide

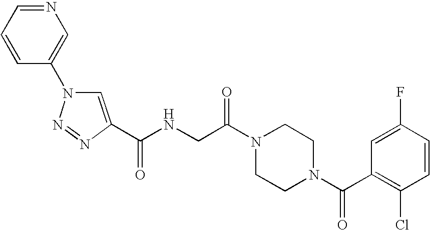

DIPEA (149 mg, 0.2 mL, 1.1 mmol) followed by HOBT (42.9 mg, 0.31 mmol) and EDCI (110 mg, 0.57 mmol) were added to a stirred solution of 1-pyridin-3-yl-1H-[1,2,3]triazole-4-carboxylic acid (55 mg, 0.29 mmol) (prepared by a procedure similar to that described in Steps 1-3 of General Scheme 1) in DMF (2 mL) at room temperature. After 2 minutes of stirring, 2-amino-1-[4-(2-chloro-5-fluoro-benzoyl)-piperazin-1-yl]-ethanone hydrochloride (97.2 mg, 0.29 mmol) (prepared according to a procedure similar to that described in Synthesis Procedure 1, using 2-chloro-5-fluoro benzoic acid (Aldrich, St. Louis, Mo.) as a starting material) was added and the resulting mixture was stirred at room temperature overnight. Cold water was to the reaction flask and the resulting precipitate was purified by recrystallisation from a mixture of dichloromethane and hexane to afford 97 mg (71%) of 1-pyridin-3-yl-1H-[1,2,3]triazole-4-carboxylic acid {2-[4-(2-chloro-5-fluoro-benzoyl)-piperazin-1-yl]-2-oxo-ethyl}-amide. LCMS Purity: 100%. ¹H NMR (DMSO-d₆): δ 9.45 (s, 1H), 9.2 (d, 1H), 8.75 (dd, 1H), 8.6-8.5 (t, 1H), 8.4 (m, 1H), 7.7 (q, 1H), 7.6 (m, 1H), 7.5-7.3 (m, 2H), 4.3-4.15 (dd, 2H), 3.8-3.6 (m, 4H), 3.3-3.15 (m, 214).

### Example 11

### 1-[4-(5-Fluoro-2-trifluoromethyl-benzoyl)-piperazin-1-yl]-2-(4-thiophen-3-yl-benzylamino)-ethanone hydrochloride

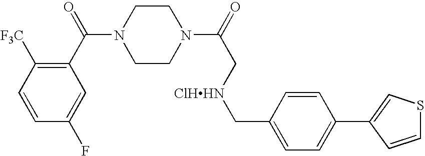

### Step 1

### Synthesis of 4-thiophen-3-yl-benzaldehyde

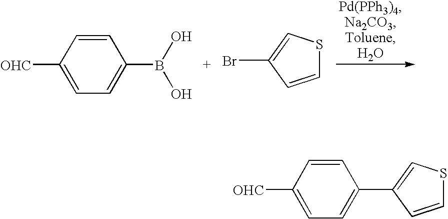

Sodium carbonate (424 mg, 4.0 mmol) followed, after 5 minutes, by 4-formyl phenyl boronic acid (600 mg, 0.4 mmol) and 3-bromo-thiophene (326 mg, 2.0 mmol) were added to a degassed mixture of toluene (12 mL) and water (3 mL). The reaction mixture was degassed for a further 5 minutes. Tetrakis palladium triphenylphosphine (231.1 mg, 0.2 mmol) was then added and the reaction mixture was degassed for a further 5 minutes, then heated to reflux for 3 hrs. The mixture was then diluted with ethyl acetate and partitioned with water. The ethyl acetate layer was washed with brine solution, dried over sodium sulphate and concentrated under reduced pressure to afford a crude product which was purified by column chromatography using silica gel 60-120 mesh (3% ethyl acetate in hexane) to afford 210 mg (56%) of 4-thiophen-3-yl-benzaldehyde.

### Step 2

### Synthesis of 1-[4-(5-Fluoro-2-trifluoromethyl-benzoyl)-piperazin-1-yl]-2-(4-thiophen-3-yl-benzylamino)-ethanone

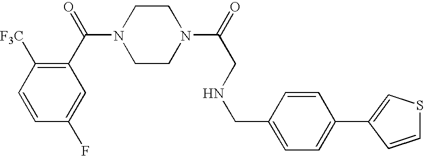

Sodium triacetoxy borohydride (67.8 mg, 0.32 mmol) was added portion wise to a stirred cold (−5° C.) solution of 4-thiophen-3-yl-benzaldehyde (50 mg, 0.26 mmol) and 2-amino-1-[4-(5-fluoro-2-trifluoromethyl-benzoyl)-piperazin-1-yl]ethanone hydrochloride (97.9 mg, 0.26 mmol) (prepared according to a procedure similar to that described in Synthesis Procedure 1, using 5-fluoro-2-(trifluoromethyl)benzoic acid (Aldrich, St. Louis, Mo.) as a starting material) in DCE (8 mL) and stirring was continued at ambient temperature overnight. Cold water was added to the reaction mixture followed by saturated sodium bicarbonate solution and the product was extracted with dichloromethane. The organic layer was collected, dried over sodium sulfate and concentrated under reduced pressure. Purification of the residue by preparative HPLC afforded 50 mg (19%) of 1-[4-(5-fluoro-2-trifluoromethyl-benzoyl)-piperazin-1-yl]-2-(4-thiophen-3-yl-benzylamino)-ethanone. LCMS Purity: 96.79%.

### Step 3

### Synthesis of 1-[4-(5-Fluoro-2-trifluoromethyl-benzoyl)-piperazin-1-yl]-2-(4-thiophen-3-yl-benzylamino)-ethanone hydrochloride

1-[4-(5-fluoro-2-trifluoromethyl-benzoyl)-piperazin-1-yl]-2-(4-thiophen-3-yl-benzylamino)-ethanone (50 mg, 0.09 mmol) in ether-HCl solution (5 ml) was stirred at room temperature for 1 hr. The reaction mixture was then concentrated and the residue was washed with ether to afford 36 mg (67%) of 1-[4-(5-fluoro-2-trifluoromethyl-benzoyl)-piperazin-1-yl]-2-(4-thiophen-3-yl-benzylamino)-ethanone hydrochloride. LCMS Purity: 96.34%. ¹H NMR (DMSO-d₆): δ 9.2 (s, 1H), 8.0-7.9 (m, 2H), 7.85-7.74 (m, 2H), 7.7-7.5 (m, 6H), 4.2-4.0 (m, 4H), 3.8-3.4 (m, 5H), 3.3-3.0 (m, 3H).

### Example 12

### 2-[(Biphenyl-4-ylmethyl)-amino]-1-[4-(3,4,5-trifluoro-benzoyl)-piperazin-1-yl]-ethanone hydrochloride

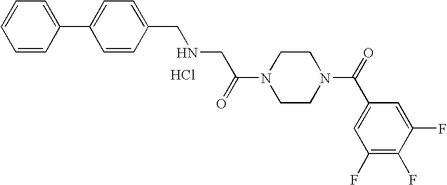

### Step 1

### Synthesis of 2-[(Biphenyl-4-ylmethyl)-amino]-1-[4-(3,4,5-trifluoro-benzoyl)-piperazin-1-yl]-ethanone

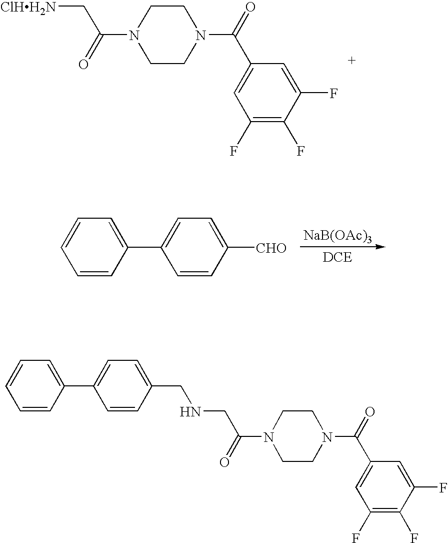

Sodium triacetoxy borohydride (313.7 mg, 1.48 mmol) was added portion wise to a stirred cold (0-5° C.) solution of biphenyl-4-carbaldehyde (134.8 mg, 0.74 mmol) and 2-amino-1-[4-(3,4,5-trifluoro-benzoyl)-piperazin-1-yl]-ethanone hydrochloride (250 mg, 0.74 mmol) (prepared according to a procedure similar to that described in Synthesis Procedure 1, using 3,4,5-trifluoro benzoic acid (Aldrich, St. Louis, Mo.) as a starting material) in DCE (7 mL) and stirring was continued at ambient temperature overnight. Cold water was added to the reaction mixture followed by saturated sodium bicarbonate solution and the product was extracted with dichloromethane. The organic layer was dried over sodium sulfate, concentrated under reduced pressure and the residue was purified by preparative HPLC to afford 90 mg (26%) of 1-[4-(5-fluoro-2-trifluoromethyl-benzoyl)-piperazin-1-yl]-2-(4-thiophen-3-yl-benzylamino)-ethanone.

### Step 2

### Synthesis of 2-[(Biphenyl-4-ylmethyl)-amino]-1-[4-(3,4,5-trifluoro-benzoyl)-piperazin-1-yl]-ethanone hydrochloride

A solution of 2-[(biphenyl-4-ylmethyl)-amino]-1-[4-(3,4,5-trifluoro-benzoyl)-piperazin-1-yl]-ethanone (50 mg, 0.09 mmol) in ether-HCl (5 ml) was stirred at room temperature for 2 hrs. The reaction mixture was then concentrated and the residue was washed with ether to afford 54 mg (59%) of 2-[(biphenyl-4-ylmethyl)-amino]-1-[4-(3,4,5-trifluoro-benzoyl)-piperazin-1-yl]-ethanone hydrochloride. LCMS Purity: 95.3%. ¹H NMR (DMSO-d₆): δ 9.5 (s, 2H), 7.8±7.67 (m, 4H), 7.66-7.6 (d, 2H), 7.55-7.43 (m, 4H), 7.43-7.35 (m, 1H), 4.23±4.02 (m, 4H), 3.68-3.5 (m, 5H), 3.5-3.3 (m, 4H).

### Example 13

### 5-(3-Fluoro-phenyl)-1H-pyrazole-3-carboxylic acid {2-[4-(5-fluoro-2-trifluoromethyl-benzoyl)-piperazin-1-yl]-2-oxo-ethyl}-amide

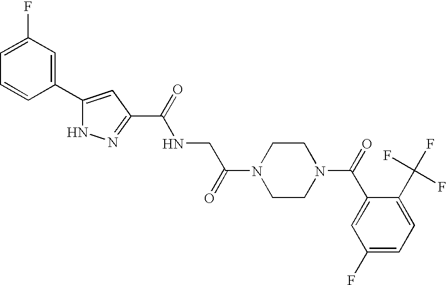

DIPEA (142 mg, 0.19 mL, 1.1 mmol) followed by HOBT (44.7 mg, 0.33 mmol) and EDCI (63.5 mg, 0.33 mmol) were added to a stirred solution of 5-(3-fluoro-phenyl)-1H-pyrazole-3-carboxylic acid (68 mg, 0.31 mmol) (prepared according to a procedure similar to that described in Synthesis Procedure 4, Steps 1-3 using 3′-fluoroacetophenone (Aldrich, St. Louis, Mo.) as a starting material) in DMF (2 mL). After 2 minutes of stirring, 2-amino-1-[4-(5-fluoro-2-trifluoromethyl-benzoyl)-piperazin-1-yl]-ethanone hydrochloride (119 mg, 0.31 mmol) (prepared according to a procedure similar to that described in Synthesis Procedure 1, using 5-fluoro-2-(trifluoromethyl)benzoic acid (Aldrich, St. Louis, Mo.) as a starting material) was added and the resulting mixture was stirred at room temperature overnight. The reaction mixture was then partitioned between ethyl acetate and water. The organic layer was washed with saturated brine solution, dried over sodium sulphate and concentrated. The residue was purified by recrystallisation from ethyl acetate to afford 51 mg (31%) of 5-(3-fluoro-phenyl)-1H-pyrazole-3-carboxylic acid {2-[4-(5-fluoro-2-trifluoromethyl-benzoyl)-piperazin-1-yl]-2-oxo-ethyl}-amide. LCMS-purity: 91%, ¹H NMR (DMSO-d₆): δ 13.8 (s, 1H), 8.1 (m, 1H), 7.9 (m, 1H), 7.42 (m, 5H), 7.2 (m, 2H), 4.1 (m, 2H), 3.4 (m, 6H), 3.1 (m, 2H).

### Example 14

### 5-(4-Fluoro-phenyl)-1H-pyrazole-3-carboxylic acid {2-[4-(5-fluoro-2-trifluoromethyl-benzoyl)-piperazin-1-yl]-2-oxo-ethyl}-amide

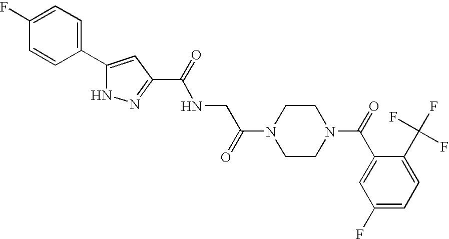

DIPEA (131 mg, 0.17 mL, 1.0 mmol) followed by HOBT (41.3 mg, 0.3 mmol) and EDCI (58.6 mg, 0.3 mmol) were added to a stirred solution of 5-(4-fluoro-phenyl)-1H-pyrazole-3-carboxylic acid (60 mg, 0.29 mmol) (prepared according to a procedure similar to that described in Synthesis Procedure 4, Steps 1-3, using 3′-fluoroacetophenone (Aldrich, St. Louis, Mo.) as a starting material) in DMF (2 mL) at room temperature. After 2 minutes, 2-amino-1-[4-(5-fluoro-2-trifluoromethyl-benzoyl)-piperazin-1-yl]-ethanone hydrochloride (108 mg, 0.3 mmol) (prepared according to a procedure similar to that described in Synthesis Procedure 1, using 5-fluoro-2-(trifluoromethyl)benzoic acid (Aldrich, St. Louis, Mo.) as a starting material) was added and the resulting mixture was stirred at room temperature overnight. The reaction mixture was then partitioned between cold water and ethyl acetate. The organic layer was washed with saturated brine solution, dried over sodium sulphate and purified by column chromatography using 60-120 silica gel and 20% EtOAc in Hexane to afford 86 mg (57%) of 5-(4-fluoro-phenyl)-1H-pyrazole-3-carboxylic acid {2-[4-(5-fluoro-2-trifluoromethyl-benzoyl)-piperazin-1-yl]-2-oxo-ethyl}-amide. LCMS-purity: 98.04%, ¹H NMR (DMSO-d₆): δ 13.7 (s, 1H), 8.1 (t, 1H), 7.8 (m, 3H), 7.5 (m, 2H), 7.2 (m, 2H), 7.1 (s, 1H), 4.1 (m, 2H), 3.4 (m, 6H), 3.2 (m, 2H).

### Example 15

### 4-Phenyl-pyrazole-1-carboxylic acid {2-oxo-2-[4-(4-trifluoromethyl-pyridine-3-carbonyl)-piperazin-1-yl]-ethyl}-amide

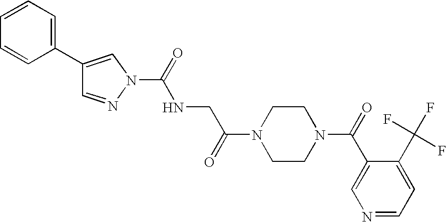

A mixture of 4-phenyl-1H-pyrazole hydrochloride (70 mg, 0.38 mmol) (prepared according to Synthesis Procedure 4) DIPEA (110.2 mg, 0.15 ml, 0.85 mmol) and DCM (3.5 ml) was added to a stirred solution of triphosgene (36 mg, 0.12 mmol) in DCM (8 mL) at room temperature. After 30 minutes, a mixture of 2-amino-1-[4-(4-trifluoromethyl-pyridine-3-carbonyl)-piperazin-1-yl]-ethanone hydrochloride (108 mg, 0.3 mmol) (prepared according to a procedure similar to that described in Synthesis Procedure 1, using 4-trifluoromethyl-pyridine-3-carboxylic acid (Aldrich, St. Louis, Mo.) as a starting material), DIPEA (110.2 mg, 0.15 ml, 0.85 mmol) and DCM (3.5 ml) was added and the resulting mixture was stirred at room temperature for 30 minutes. Cold water was then added, the product was extracted with EtOAc and the organic layer was washed with brine. The organic phase was dried over Na₂SO₄ and concentrated under reduced pressure. The resulting residue was purified by column chromatography (using silica gel of 60-120 mesh and 50% EtOAc in Hexane as eluent) to afford 65 mg (34%) 4-phenyl-pyrazole-1-carboxylic acid {2-oxo-2-[4-(4-trifluoromethyl-pyridine-3-carbonyl)-piperazin-1-yl]-ethyl}-amide. LCMS Purity: 98.97%. ¹H NMR (DMSO-d₆): δ 8.95 (d, 1H), 8.9 (d, 1H), 8.8 (s, 1H), 8.42 (t, 1H), 8.35 (s, 1H), 7.9 (m, 1H), 7.8-7.7 (d, 2H), 7.45-7.35 (t, 2H), 7.3 (t, 1H), 4.3-4.14 (dd, 2H), 3.8-3.4 (m, 6H), 3.3-3.2 (m, 2H).

### Example 16

### 5-(4-Fluoro-phenyl)-isoxazole-3-carboxylic acid {2-[4-(5-fluoro-2-trifluoromethyl-benzoyl)-piperazin-1-yl]-2-oxo-ethyl}-amide

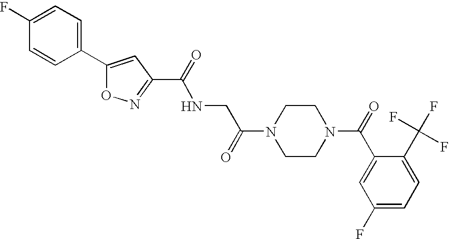

DIPEA (131 mg, 0.17 mL, 1.0 mmol) followed by HOBT (41.3 mg, 0.3 mmol) and EDCI (58.6 mg, 0.3 mmol) were added to a stirred solution of 5-(4-fluoro-phenyl)-isoxazole-3-carboxylic acid (60 mg, 0.29 mmol) (prepared according to a procedure similar to that described in Synthesis Procedure 3, Steps 1-3, using 4′-fluoracetophenone (Aldrich, St. Louis, Mo.) as a starting material) in DMF (2 mL) at room temperature. After 2 minutes, 2-amino-1-[4-(5-fluoro-2-trifluoromethyl-benzoyl)-piperazin-1-yl]-ethanone hydrochloride salt (108 mg, 0.3 mmol) (prepared according to a procedure similar to that described in Synthesis Procedure 1, using 5-fluoro-2-(trifluoromethyl)benzoic acid (Aldrich, St. Louis, Mo.) as a starting material) was added and the resulting mixture was stirred at room temperature overnight. Cold water was then added and the product was extracted with ethyl acetate. The organic layer was washed with saturated brine solution and dried over sodium sulphate. The organic layer was then concentrated and the product purified by recrystallisation from a mixture of 25% ethyl acetate in hexane to afford 30 mg (20%) of 5-(4-fluoro-phenyl)-isoxazole-3-carboxylic acid {2-[4-(5-fluoro-2-trifluoromethyl-benzoyl)-piperazin-1-yl]-2-oxo-ethyl}-amide. LCMS-purity: 91.32%, ¹H NMR (CDCl₃): δ 7.7 (m, 4H), 7.28 (m, 1H), 7.18 (m, 2H), 7.06 (m, 1H), 6.9 (m, 1H), 4.32 (m, 1H), 4.2 (m, 1H), 3.9 (m, 2H), 3.7 (m, 2H), 3.5 (m, 2H), 3.4 (m, 1H), 3.2 (m, 2H).

### Example 17

### N-{2-[4-(5-Fluoro-2-trifluoromethyl-benzoyl)-piperazin-1-yl]-2-oxo-ethyl}-6-pyrrolidin-1-yl-nicotinamide

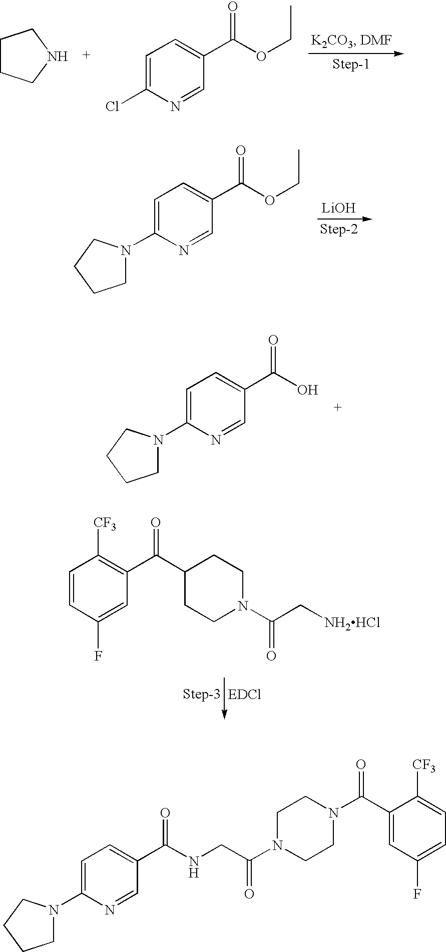

### Step 1

### Synthesis of 6-Pyrrolidin-1-yl-nicotinic acid ethyl ester

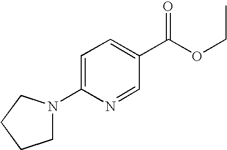

Potassium carbonate (3.89 g, 28 mmol) was added to a stirred solution of pyrrolidine (1.0 g, 14 mmol) and 6-chloronicotinic acid ethyl ester (2.87 g, 15.5 mmol) in DMF (10 mL) and the resulting mixture was stirred with heating at 60° C. overnight. Cold water was then added to and the resulting precipitate was dried to afford 498 mg (80%) of 6-pyrrolidin-1-yl-nicotinic acid ethyl ester. LCMS-purity: 97%.

### Step 2

### Synthesis of 6-Pyrrolidin-1-yl-nicotinic acid

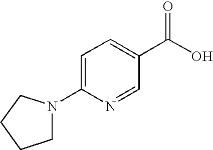

LiOH (380 mg, 0.9 mmol) was added to a stirred solution of 6-pyrrolidin-1-yl-nicotinic acidethyl ester (1 g, 4.5 mmol) in THF: MeOH: H₂O (2:6:1, 18 mL) and the resulting mixture was stirred for 30 minutes at room temperature. The mixture was then concentrated under reduced pressure and the residue was diluted with cold water, acidified with 10% aqueous HCl and filtered. The residue was collected to afford 736 mg (84%) of 6-pyrrolidin-1-yl-nicotinic acid. LCMS Purity: 94.28%.

### Step 3

### Synthesis of N-{2-[4-(5-Fluoro-2-trifluoromethyl-benzoyl)-piperazin-1-yl]-2-oxo-ethyl}-6-pyrrolidin-1-yl-nicotinamide

DIPEA (141 mg, 0.19 mL, 1.0 mmol) followed by HOBT (59 mg, 0.43 mmol) and EDCI (83.5 mg, 0.43 mmol) were added to a stirred solution of 6-pyrrolidin-1-yl-nicotinic acid (70 mg, 0.36 mmol) in DMF (1 mL) at room temperature. After 2 minutes, 2-amino-1-[4-(5-fluoro-2-trifluoromethyl-benzoyl)-piperazin-1-yl]ethanone hydrochloride (prepared according to a procedure similar to that described in Synthesis Procedure 1, using 5-fluoro-2-(trifluoromethyl)benzoic acid (Aldrich, St. Louis, Mo.) as a starting material) (161 mg, 0.43 mmol) was added and the resulting mixture was stirred at room temperature overnight. Cold water was then added and the precipitate thus formed was collected and washed with hexane to afford 70 mg (38%) of N-{2-[4-(5-fluoro-2-trifluoromethyl-benzoyl)-piperazin-1-yl]-2-oxo-ethyl}-6-pyrrolidin-1-yl-nicotinamide. LCMS-purity: 87.7%, ¹H NMR (CDCl₃): δ 8.65 (d, 1H), 7.85 (m, 1H), 7.75 (m, 1H), 7.24 (m, 1H), 7.1-6.96 (m, 2H), 4.38-4.1 (m, 2H), 4.05-3.89 (m, 1H), 3.75-3.65 (m, 2H), 3.6-3.35 (m, 7H), 3.3-3.15 (m, 2H), 2.1-1.95 (m, 4H).

### Example 18

### 5-(3-Hydroxy-phenyl)-1H-pyrazole-3-carboxylic acid {2-[4-(5-fluoro-2-trifluoromethyl-benzoyl)-piperazin-1-yl]-2-oxo-ethyl}-amide

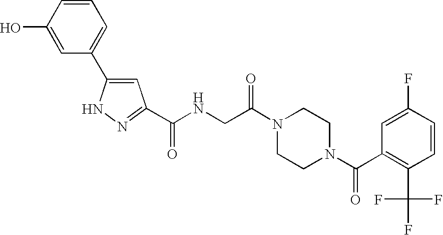

10% Pd/C (40 mg) was added to a stirred solution of 5-(3-benzyloxy-phenyl)-1H-pyrazole-3-carboxylic acid {2-[4-(5-fluoro-2-trifluoromethyl-benzoyl)-piperazin-1-yl]-2-oxo-ethyl}-amide (142 mg, 0.23 mmol) (prepared according to a procedure similar to that described in Synthesis Procedure 3, Steps 1-4-b, using 1-(3-hydroxyphenyl)ethanone (Aldrich, St. Louis, Mo.) as a starting material) in MeOH (50 mL) and the resulting mixture was hydrogenated for 2 hrs. The mixture was then filtered over celite. The residue was washed with MeOH and the filtrate was concentrated under reduced pressure. The resulting residue was purified by preparative HPLC to afford 41 mg (34%) of 5-(3-hydroxy-phenyl)-1H-pyrazole-3-carboxylic acid {2-[4-(5-fluoro-2-trifluoromethyl-benzoyl)-piperazin-1-yl]-2-oxo-ethyl}-amide. LCMS Purity: 95.23%. ¹H NMR (DMSO-d₆): δ 8.2 (m, 1H), 7.9 (m, 1H), 7.48 (m, 2H), 7.24 (m, 2H), 6.96 (m, 1H), 6.72 (m, 1H), 4.1 (m, 2H), 3.46 (m, 6H), 3.04 (m, 2H).

### Example 19

### 5-(4-Hydroxy-phenyl)-1H-pyrazole-3-carboxylic acid {2-[4-(5-fluoro-2-trifluoromethyl-benzoyl)-piperazin-1-yl]-2-oxo-ethyl}-amide

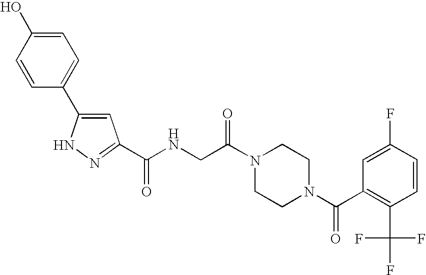

10% Pd/C (40 mg) was added to a stirred solution of 5-(4-benzyloxy-phenyl)-1H-pyrazole-3-carboxylic acid {2-[4-(5-fluoro-2-trifluoromethyl-benzoyl)-piperazin-1-yl]-2-oxo-ethyl}-amide (149 mg, 0.24 mmol) (prepared according to a procedure similar to that described in Synthesis Procedure 3, using 4′-hydroxyacetophenone (Aldrich, St. Louis, Mo.) as a starting material) in MeOH (50 mL) and the resulting mixture was hydrogenated for 2 hrs. The mixture was then filtered over celite. The residue was washed with MeOH and the filtrate was concentrated under reduced pressure and purified by preparative HPLC to afford 31 mg (24%) of 5-(4-hydroxy-phenyl)-1H-pyrazole-3-carboxylic acid {2-[4-(5-fluoro-2-trifluoromethyl-benzoyl)-piperazin-1-yl]-2-oxo-ethyl}-amide. LCMS Purity: 95.23%. ¹H NMR (DMSO-d₆): δ 8.06 (s, 1H), 7.9 (m, 1H), 7.5 (m, 4H), 6.88 (m, 1H), 6.78 (d, 2H), 4.1 (m, 2H), 3.5 (m, 6H), 3.14 (m, 2H).

### Example 20

### 1-Phenyl-1H-imidazole-4-carboxylic acid {2-[4-(2-chloro-5-fluoro-benzoyl)-piperazin-1-yl]-2-oxo-ethyl}-amide

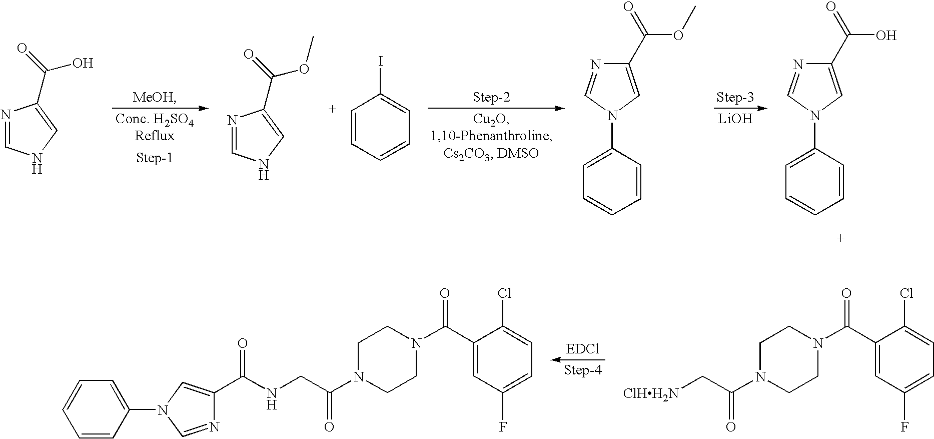

### Step 1

### Synthesis of 1H-imidazole-4-carboxylic acid methyl ester

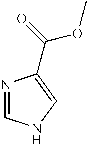

A stirred solution of 1H-imidazole-4-carboxylic acid (1.0 g, 8.9 mmol), concentrated sulfuric acid (1 mL) in MeOH (30 mL) was heated to reflux overnight. The mixture was then concentrated under reduced pressure and partitioned between cold water and ethyl acetate. The organic layer was collected and concentrated under reduced pressure to afford 1 g (89%) of 1H-imidazole-4-carboxylic acid methyl ester.

### Step 2

### Synthesis of 1-Phenyl-1H-imidazole-4-carboxylic acid methyl ester

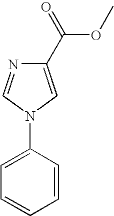

A mixture of 1H-imidazole-4-carboxylic acid methyl ester (25 mg, 1.98 mmol), cuprous oxide (141 mg, 0.99 mmol), iodobenzene (809 mg, 3.9 mmol), 1,10-phenanthroline (357 mg, 1.98 mmol) and cesium carbonate (1.93 g, 5.94 mmol) in DMSO (2 mL) was subjected to reaction in microwave reactor (time: 5 min, temp: 90° C., power: zero). The reaction mixture was filtered over celite and the filtrate was concentrated under reduced pressure which was partitioned between cold water and ethyl acetate. The organic layer was concentrated under reduced pressure and the resulting residue was purified by column chromatography (using 60-120 silica gel and 50% ethyl acetate in hexane as eluent) to afford 200 mg (50%) of 1-phenyl-1H-imidazole-4-carboxylic acid methyl ester

### Step 3

### Synthesis of 1-Phenyl-1H-imidazole-4-carboxylic acid

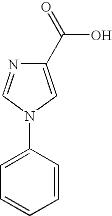

To a stirred solution of 1-phenyl-1H-imidazole-4-carboxylic acid methyl ester (200 mg, 0.98 mmol) in a mixture of THF (3 mL), MeOH (3 mL), H₂O (2 mL) was added, LiOH.H₂O (249 mg, 5.9 mmol) and stirring was continued at ambient temperature for 2 hr. The reaction mixture was concentrated under reduced pressure. Cold water was then added to the residue which was then acidified with 10% aqueous HCl. The product was extracted with ethyl acetate. The organic layer was dried over sodium sulfate and concentrated under reduced pressure to afford 105 mg (56%) of 1-phenyl-1H-imidazole-4-carboxylic acid.

### Step 4

### Synthesis of 1-Phenyl-1H-imidazole-4-carboxylic acid {2-[4-(2-chloro-5-fluoro-benzoyl)-piperazin-1-yl]-2-oxo-ethyl}-amide

DIPEA (110 mg, 0.85 mmol) followed by HOBT (31.6 mg, 0.23 mmol) and EDCI (81.5 mg, 0.42 mmol) were added to a stirred solution of 1-phenyl-1H-imidazole-4-carboxylic acid (40 mg, 0.21 mmol) in DMF (2 mL) at room temperature. After 2 minutes, 2-amino-1-[4-(2-chloro-5-fluoro-benzoyl)-piperazin-1-yl]-ethanone hydrochloride (prepared according to a procedure similar to that described in Synthesis Procedure 1, using 2-chloro-5-fluoro benzoic acid (Aldrich, St. Louis, Mo.) as a starting material) (71.4 mg, 0.21 mol) was added and the resulting mixture was stirred at ambient temperature overnight. The reaction mixture was diluted with cold water and the product was extracted with dichloromethane. The organic layer was dried over sodium sulfate and concentrated under reduced pressure. The resulting residue was purified by recrystallisation from a mixture of methanol and diethyl ether to afford 63 mg (63%) of 1-phenyl-1H-imidazole-4-carboxylic acid {2-[4-(2-chloro-5-fluoro-benzoyl)-piperazin-1-yl]-2-oxo-ethyl}-amide. LCMS Purity: 97.53%. ¹H NMR (DMSO-d₆): δ 8.4 (s, 1H), 8.3 (s, 1H), 8.1-8.0 (t, 1H), 7.8-7.7 (d, 2H), 7.65-7.5 (m, 3H), 7.5-7.3 (m, 3H), 4.3-4.1 (m, 2H), 3.8-3.56 (m, 4H), 3.5 (m, 2H), 3.3-3.15 (m, 2H).

### Example 21

3-[1,3,4]Oxadiazol-2-yl-N-{2-oxo-2-[4-(2-trifluoromethyl-benzoyl)-piperazin-1-yl]-ethyl}-benzamide

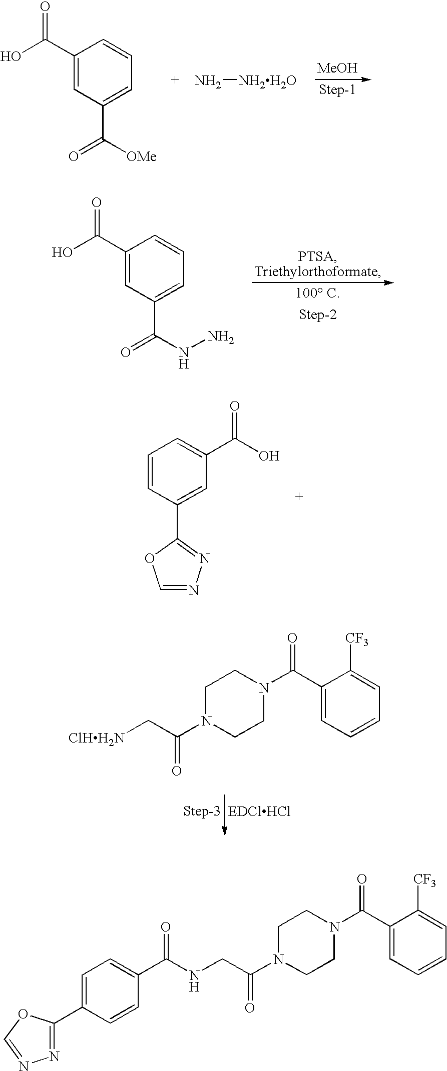

### Step 1

### Synthesis of 3-Hydrazinocarbonyl-benzoic acid

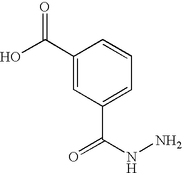

Hydrazine hydrate (2.42 g, 48.5 mmol) was added to a solution of isophthalic acid monomethyl ester (250 mg, 13.87 mmol) in MeOH (10 mL) and the resulting mixture was heated to reflux for 4 hr. The reaction mixture was then concentrated to afford 350 mg of 3-hydrazinocarbonyl-benzoic acid which was used for the next step without further purification.

### Step 2

### Synthesis of 3-[1,3,4]Oxadiazol-2-yl-benzoic acid

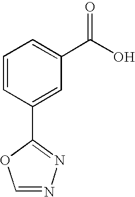

p-Toluene sulfonic acid (15.8 mg, 0.083 mmol) was added to a solution of 3-hydrazinocarbonyl-benzoic acid (150 mg, 0.83 mmol) in triethylorthoformate (2.37 g, 7.5 mL, 16.6 mmol) and stirring was continued with heating at 60° C. for 4 hrs. The reaction mixture was diluted with water and the resulting precipitate was isolated to afford 80 mg (51%) of 3-[1,3,4]oxadiazol-2-yl-benzoic acid.

### Step 3

### Synthesis of 3-[1,3,4]Oxadiazol-2-yl-N-{2-oxo-2-[4-(2-trifluoromethyl-benzoyl)-piperazin-1-

DIPEA (154 mg, 0.2 mL, 1.19 mmol) was added drop wise to 3-[1,3,4]oxadiazol-2-yl)-benzoic acid (65 mg, 0.34 mmol) in DMF (3 mL). EDCI (98.2 mg, 0.51 mmol) and HOBT (50 mg, 0.37 mmol) were then added. After 2 minutes, 2-amino-1-[4-(2-trifluoromethyl-benzoyl)-piperazin-1-yl]-ethanone hydrochloride (132 mg, 0.37 mmol) (prepared according to a procedure similar to that described in Synthesis Procedure 1, Steps 1A-E, using HCl/MeOH or 1,4-dioxane in place of trifluoroacetic acid in the last step) was added. The resulting mixture was stirred at room temperature overnight. Cold water was then added and the resulting precipitate was isolated by filtration to afford 90 mg (54%) of 4-[1,3,4]oxadiazol-2-yl-N-{2-oxo-2-[4-(2-trifluoromethyl-benzoyl)-piperazin-1-yl]-ethyl}-benzamide. LCMS Purity: 94.4%. ¹H NMR (DMSO-d₆): δ 9.4 (s, 1H), 8.9 (bs, 1H), 8.5 (s, 1H), 8.15 (dd, 2H), 7.7 (m, 4H), 7.55 (t, 1H), 4.2 (dd, 2H), 3.7 (bs, 4H), 3.5 (s, 1H), 3.1 (d, 3H).

### Example 22

### 5-(2-Amino-phenyl)-isoxazole-3-carboxylic acid {2-oxo-2-[4-(2-trifluoromethyl-benzoyl)-piperazin-1-yl]-ethyl}-amide

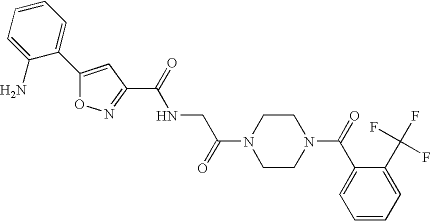

### Step 1

### Synthesis of 5-(2-Nitro-phenyl)-isoxazole-3-carboxylic acid {2-oxo-2-[4-(2-trifluoromethyl-benzoyl)-piperazin-1-yl]-ethyl}-amide

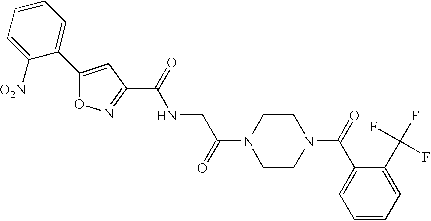

DIPEA (220 mg, 0.3 mL, 1.7 mmol) was added to a stirred solution of 5-(2-nitro-phenyl)-isoxazole-3-carboxylic acid (100 mg, 0.43 mmol) (prepared according to a procedure similar to that described in Synthesis Procedure 3, Steps 1-3, using 2′-nitroacetophenone (Aldrich, St. Louis, Mo.) as a starting material) in DMF (3 mL), HOBT (72 mg, 0.53 mmol) and EDCI (102 mg, 0.53 mmol) were then added at room temperature. After 2 minutes, 2-amino-1-[4-(2-trifluoromethyl-benzoyl)-piperazin-1-yl]-ethanone hydrochloride (150 mg, 0.43 mmol) (prepared according to a procedure similar to that described in Synthesis Procedure 1, Steps 1A-E, using HCl/MeOH or 1,4-dioxane in place of trifluoroacetic acid in the last step) was added and the resulting mixture was stirred at room temperature overnight. Cold water was then added and the product was extracted with ethyl acetate. The organic layer was washed with brine, dried over Na₂SO₄ and concentrated under reduced pressure. The resulting residue was purified by column chromatography (using 60-120 silica gel and 75% EtOAc in hexane as eluent) to afford 110 mg (49%) of 5-(2-nitro-phenyl)-isoxazole-3-carboxylic acid {2-oxo-2-[4-(2-trifluoromethyl-benzoyl)-piperazin-1-yl]-ethyl}-amide. LCMS Purity: 72.3%.

### Step 2

### Synthesis of 5-(2-Amino-phenyl)-isoxazole-3-carboxylic acid {2-oxo-2-[4-(2-trifluoromethyl-benzoyl)-piperazin-1-yl]-ethyl}-amide

10% Pd/C (25 mg) was added to a stirred solution of 5-(2-nitro-phenyl)-isoxazole-3-carboxylic acid {2-oxo-2-[4-(2-trifluoromethyl-benzoyl)-piperazin-1-yl]-ethyl}-amide (110 mg, 0.2 mmol) in MeOH (50 mL) and the resulting mixture was stirred under an atmosphere of hydrogen for 1 hr. The mixture was then filtered over a bed of celite. The celite was washed with MeOH and the filtrate was concentrated under reduced pressure to afford crude 5-(2-amino-phenyl)-isoxazole-3-carboxylic acid {2-oxo-2-[4-(2-trifluoromethyl-benzoyl)-piperazin-1-yl]-ethyl}-amide. The crude residue was purified by preparative HPLC to afford 23 mg (40%) of 542-amino-phenyl)-isoxazole-3-carboxylic acid {2-oxo-2-[4-(2-trifluoromethyl-benzoyl)-piperazin-1-yl]-ethyl}-amide. LCMS Purity: 100%. ¹H NMR (DMSO-d₆): δ 8.62 (t, 1H), 7.64 (m, 3H), 7.54 (m, 2H), 7.1 (m, 2H), 6.82 (m, 1H), 6.64 (m, 1H), 5.7 (s, 1H), 4.1 (m, 2H), 3.5 (m, 4H), 3.4 (m, 2H), 3.1 (m, 2H).

### Example 23

### Naphthalene-2-carboxylic acid {2-[4-(5-fluoro-2-trifluoromethyl-benzoyl)-piperazin-1-yl]-2-oxo-ethyl}-amide

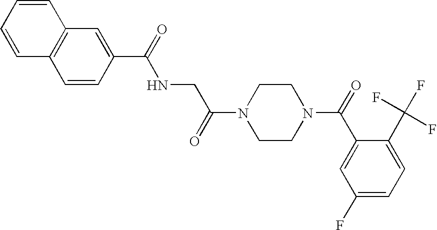

DIPEA (269 mg, 0.36 mL, 2.08 mmol) was added to a stirred solution of naphthalene-2-carboxylic acid (80 mg, 0.46 mmol) in DMF (2 mL). HOBT (69 mg, 0.51 mmol) and EDCI (177 mg, 0.92 mmol) were then added at room temperature. After 2 minutes, 2-amino-1-[4-(2-trifluoromethyl-benzoyl)-piperazin-1-yl]-ethanone hydrochloride (133 mg, 0.38 mmol) (prepared according to a procedure similar to that described in Synthesis Procedure 1, using 5-fluoro-2-(trifluoromethyl)benzoic acid (Aldrich, St. Louis, Mo.) as a starting material) was added and the resulting mixture was stirred at room temperature overnight. Cold water was then added and the product was extracted with ethyl acetate. The organic layer was washed with saturated sodium bicarbonate solution and brine, then dried over Na₂SO₄ and concentrated under reduced pressure. The resulting residue was purified by column chromatography using (silica gel of 60-120 mesh and 40% EtOAc in Hexane as eluent) to afford 70 mg (31%) of naphthalene-1-carboxylic acid {2-[4-(5-fluoro-2-trifluoromethyl-benzoyl)-piperazin-1-yl]-2-oxo-ethyl}-amide. LCMS Purity: 87.12%. ¹H NMR (DMSO-d₆): δ 8.8-8.7 (t, 1H), 8.5-8.44 (s, 1H), 8.1-7.9 (m, 5H), 7.7-7.45 (m, 4H), 4.34-4.14 (m, 2H), 3.85-3.4 (m, 6H), 3.24-3.06 (m, 2H).

### Example 24

### 5-Phenyl-1H-pyrazole-3-carboxylic acid {2-[3,5-dimethyl-4-(2-trifluoromethyl-benzoyl)-piperazin-1-yl]-2-oxo-ethyl}-amide

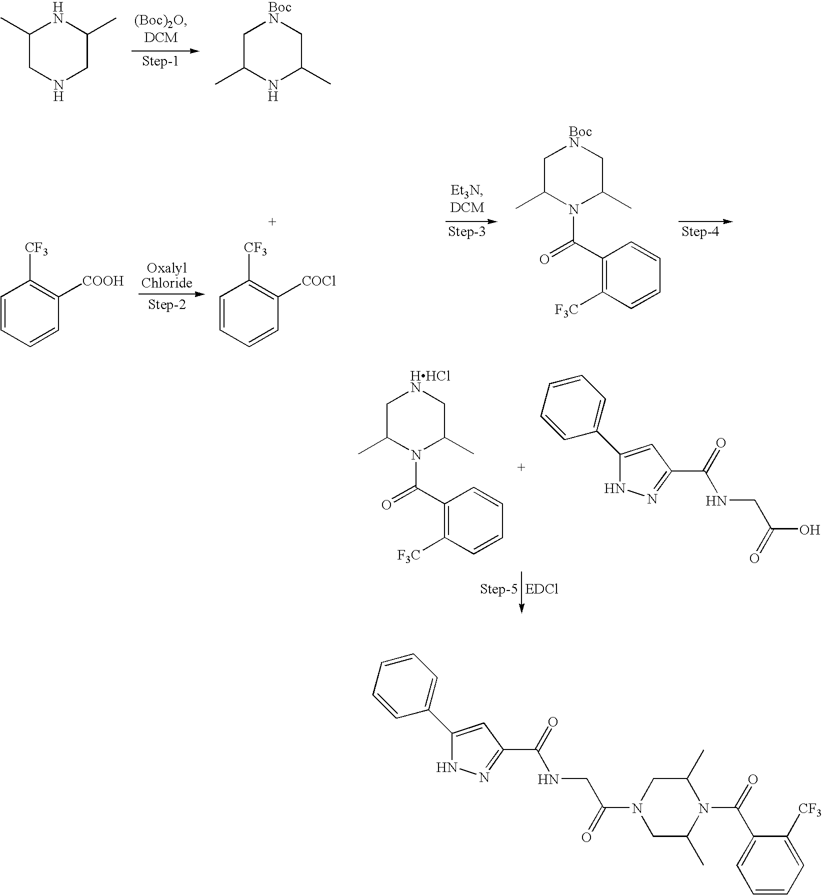

### Step 1

### Synthesis of 3,5-Dimethyl-piperazine-1-carboxylic acid tert-butyl ester

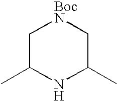

A solution of BOC-anhydride (374 mg, 1.71 mmol) in chloroform (2 mL) was added dropwise to a stirred solution of 2,5-dimethyl-piperazine (20 g, 266.5 mmol) in chloroform (2 mL) and the resulting mixture was stirred at room temperature for 4 hr. The reaction mixture was then diluted with cold water and extracted with chloroform, dried the organic layer over sodium sulfate and concentrated under reduced pressure to afford 331 mg (88.6% yield) of 3,5-dimethyl-piperazine-1-carboxylic acid tert-butyl ester. LCMS Purity: 91.3%.

### Step 2

### Synthesis of 2-Trifluoromethyl-benzoyl chloride

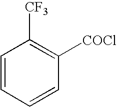

Oxalyl chloride (36.72 mg, 0.29 mmol) was added to a stirred solution of 2-trifluoromethyl-benzoic acid (50 mg, 0.26 mmol) in DCM (3 mL), followed by the addition of four drops of DMF. The resulting mixture was stirred at room temperature for 1 hr. The reaction mixture was then concentrated under reduced pressure to afford 95.6 mg (100%) of 2-trifluoromethyl-benzoyl chloride, which was used in the next step without further purification.

### Step 3

### Synthesis of 3,5-Dimethyl-4-(2-trifluoromethyl-benzoyl)-piperazine-1-carboxylic acid tert-butyl ester

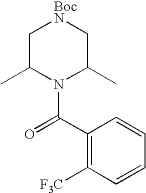

Triethyl amine (55.6 mg, 0.07 mL, 0.55 mmol) was added to a stirred solution of 2,5-dimethyl-piperazine-1-carboxylic acid tert-butyl ester (98.6 mg, 0.45 mmol) followed by 2-trifluoromethyl-benzoyl chloride (95.6 mg, 0.45 mmol) and the resulting mixture was stirred at room temperature for 30 minutes. The reaction mixture was concentrated under reduced pressure to get the residue. The residue thus obtained was purified by column chromatography using 60-120 silica gel and 50% ethyl acetate in hexane to afford 74 mg (41.71% yield) of 3,5-dimethyl-4-(2-trifluoromethyl-benzoyl)-piperazine-1-carboxylic acid tert-butyl ester. LCMS Purity: 96.7%.

### Step 4

### Synthesis of (2,6-Dimethyl-piperazin-1-yl)-(2-trifluoromethyl-phenyl)-methanone hydrochloride

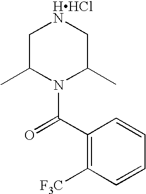

A solution of 3,5-dimethyl-4-(2-trifluoromethyl-benzoyl)-piperazine-1-carboxylic acid tert-butyl ester (70 mg, 0.18 mmol) in Dioxane. HCl (1 ml) was stirred at room temperature for 30 minutes. The reaction mixture was then concentrated and the resulting residue was washed with diethyl ether to afford 52 mg (89%) of (2,6-dimethyl-piperazin-1-yl)-(2-trifluoromethyl-phenyl)-methanone hydrochloride. LCMS Purity: 100%.

### Step 5

### Synthesis of 5-Phenyl-1H-pyrazole-3-carboxylic acid {2-[3,5-dimethyl-4-(2-trifluoromethyl-benzoyl)-piperazin-1-yl]-2-oxo-ethyl}-amide

DIPEA (27.3 mg, 0.03 6 mL, 0.21 mmol) was added to a stirred solution of [(5-phenyl-1H-pyrazole-3-carbonyl)-amino]-acetic acid (17.3 mg, 0.07 mmol) (prepared according to a procedure similar to that described in Synthesis Procedure 4, Steps 1-3 and 5-6) in DMF (1 mL). HOBT (11.42 mg, 0.08 mmol) and EDCI (16.17 mg, 0.084 mmol) were then added at room temperature. (2,6-dimethyl-piperazin-1-yl)-(2-trifluoromethyl-phenyl)-methanone hydrochloride (25 mg, 0.07 mmol) was then added and the resulting mixture was stirred at room temperature overnight. Cold water was added and the resulting precipitate was isolated by filtration to afford 20 mg (55%) of 5-phenyl-1H-pyrazole-3-carboxylic acid {2-[3,5-dimethyl-4-(2-trifluoromethyl-benzoyl)-piperazin-1-yl]-2-oxo-ethyl}-amide. LCMS Purity: 96.2%. ¹H NMR (DMSO-d₆): δ 13.65 (d, 1H), 8.2 (s, 1H), 7.8-7.74 (m, 4H), 7.72-7.62 (m, 1H), 7.6-7.54 (m, 1H), 7.5-7.42 (m, 2H), 7.4-7.26 (m, 2H), 7.12-7.26 (s, 1H), 4.8-4.65 (s, 1H), 4.35-4.05 (m, 4H), 4.0-3.65 (m, 2H), 1.45-1.1 (m, 7H), 1.0 (d, 1H).

### Example 25

### 5-Phenyl-1H-pyrazole-3-carboxylic acid {2-[5-(5-fluoro-2-trifluoromethyl-benzoyl)-2,5-diaza-bicyclo[2.2.1]hept-2-yl]-2-oxo-ethyl}-amide

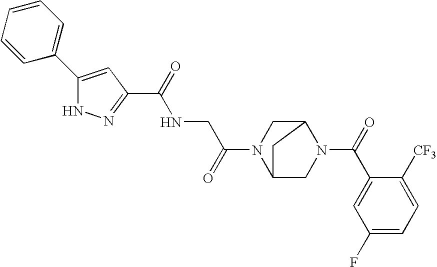

### Step 1

### Synthesis of 5-{2-[(5-Phenyl-1H-pyrazole-3-carbonyl)-amino]-acetyl}-2,5-diaza-bicyclo[2.2.1]heptane-2-carboxylic acid tert-butyl ester

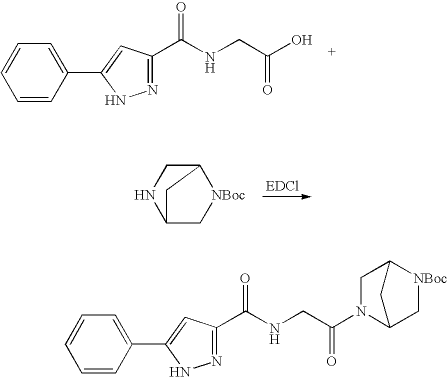

DIPEA (286 mg, 0.38 mL, 2.2 mmol) was added to a stirred solution of [(5-phenyl-1H-pyrazole-3-carbonyl)-amino]-acetic acid (136 mg, 0.55 mmol) (prepared according to a procedure similar to that described in Synthesis Procedure 4, Steps 1-3 and 5-6) in DMF (3 mL). HOBT (74.8 mg, 0.55 mmol) and EDCI (193 mg, 1.0 mmol) were then added at room temperature. After 2 minutes, 2,5-diaza-bicyclo[2.2.1]heptane-2-carboxylic acid tert-butyl ester (100 mg, 0.5 mmol) (Aldrich, St., MO) was added and the resulting mixture was stirred at room temperature overnight. Cold water was then added and the product was extracted with ethyl acetate. The organic layer was washed with saturated brine solution, dried over sodium sulfate and concentrated under reduced pressure. The resulting residue was purified by column chromatography (using 60-120 silica gel and 80% ethyl acetate in hexane as eluent) to afford 110 mg (51%) of 5-{2-[(5-phenyl-1H-pyrazole-3-carbonyl)-amino]-acetyl}-2,5-diaza-bicyclo[2.2.1]heptane-2-carboxylic acid tert-butyl ester.

### Step 2

### Synthesis of 5-Phenyl-1H-pyrazole-3-carboxylic acid [2-(2,5-diaza-bicyclo[2.2.1]hept-2-yl)-2-oxo-ethyl]-amide hydrochloride

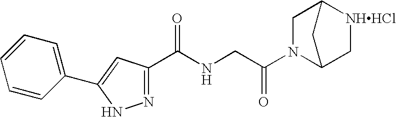

A solution of 5-{2-[(5-phenyl-1H-pyrazole-3-carbonyl)-amino]-acetyl}-2,5-diaza-bicyclo[2.2.1]heptane-2-carboxylic acid tert-butyl ester (110 mg, 0.25 mmol) in EtOAc. HCl (5 ml) was stirred at room temperature for 2 hr. The reaction mixture was then concentrated and the resulting residue was washed with diethyl ether to afford 70 mg (75%) of 5-phenyl-1H-pyrazole-3-carboxylic acid [2-(2,5-diaza-bicyclo[2.2.1]hept-2-yl)-2-oxo-ethyl]-amide hydrochloride.

### Step 3

### Synthesis of 5-Phenyl-1H-pyrazole-3-carboxylic acid {2-[5-(5-fluoro-2-trifluoromethyl-benzoyl)-2,5-diaza-bicyclo[2.2.1]hept-2-yl]-2-oxo-ethyl}-amide

DIPEA (103 mg, 0.14 mL, 0.8 mmol) was added to a stirred solution of 5-fluoro-2-trifluoromethyl-benzoic acid (41.6 mg, 0.2 mmol) in DMF (3 mL). HOBT (29.7 mg, 0.22 mmol) and EDCI (76.6 mg, 0.4 mmol) were then added at room temperature. After 2 minutes, 5-phenyl-1H-pyrazole-3-carboxylic acid [2-(2,5-diaza-bicyclo[2.2.1]hept-2-yl)-2-oxo-ethyl]amide hydrochloride (70 mg, 0.2 mmol) was added and the resulting mixture was stirred at room temperature overnight. Cold water was then added. The product was extracted with ethyl acetate and the organic layer was washed with saturated brine solution then dried over sodium sulfate. The organic layer was concentrated under reduced pressure and the resulting residue was purified by column chromatography (using 60-120 silica gel and 60% ethyl acetate in hexane as eluent) to afford 24 mg (23%) of 5-phenyl-1H-pyrazole-3-carboxylic acid {2-[5-(5-fluoro-2-trifluoromethyl-benzoyl)-2,5-diaza-bicyclo[2.2.1]hept-2-yl]-2-oxo-ethyl}-amide. LCMS Purity: 96.11%. ¹H NMR (DMSO-d₆): δ 13.7 (s, 2H), 8.6 (s, 1H), 8.2 (m, 1H), 8.1 (m, 1H), 8.0-7.85 (m, 2H), 7.85-7.7 (m, 4H), 7.7-7.6 (m, 1H), 7.6-7.3 (m, 8H), 7.1 (d, 1H), 6.6-6.5 (s, 1H), 5.0-4.7 (m, 3H), 4.2-3.9 (m, 5H), 3.7-3.4 (m, 5H), 3.0 (m, 1H), 2.0-1.4 (m, 4H).

### Example 26

### N-{2-[4-(5-Fluoro-2-trifluoromethyl-benzoyl)-piperazin-1-yl]-2-oxo-ethyl}-4-(1H-tetrazol-5-yl)-benzamide

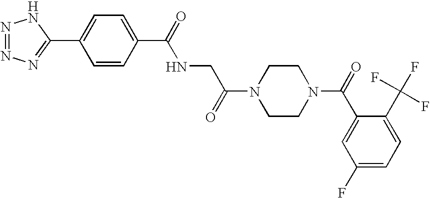

DIPEA (200 mg, 0.27 mL, 1.5 mmol) was added to a stirred solution of 4-(1H-tetrazol-5-yl)-benzoic acid (100 mg, 0.52 mmol) in DMF (2 mL). HOBT (85 mg, 0.63 mmol) and EDCI (121 mg, 0.63 mmol) were then added at room temperature. After 2 minutes, 2-amino-1-[4-(5-fluoro-2-trifluoromethyl-benzoyl)-piperazin-1-yl]-ethanone hydrochloride (233 mg, 0.63 mmol) (prepared according to a procedure similar to that described in Synthesis Procedure 1, using 5-fluoro-2-(trifluoromethyl)benzoic acid (Aldrich, St. Louis, Mo.) as a starting material) was added and the resulting mixture was stirred at room temperature overnight. Cold water was then added and the product was extracted with ethyl acetate. The organic layer was washed with saturated brine solution and dried over sodium sulfate. The organic layer collected was concentrated under reduced pressure to afford 169 mg (64%) of N-{2-[4-(5-fluoro-2-trifluoromethyl-benzoyl)-piperazin-1-yl]-2-oxo-ethyl}-4-(1H-tetrazol-5-yl)-benzamide. LCMS Purity: 91.03%. ¹H NMR (DMSO-d₆): δ 8.8 (bt, 1H), 8.2-8.0 (m, 4H), 8.0-7.9 (m, 1H), 7.6-7.5 (m, 2H), 4.3-4.1 (m, 2H), 3.8-3.4 (m, 6H), 3.3-3.0 (m, 2H).

### Example 27

### 1-[4-(5-Fluoro-2-trifluoromethyl-benzoyl)-piperazin-1-yl]-2-[(1-phenyl-1H-[1,2,3]triazol-4-ylmethyl)-amino]-ethanone hydrochloride

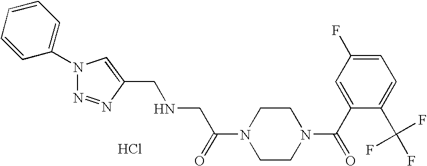

### Step 1

### Synthesis of (1-Phenyl-1H-[1,2,3]triazol-4-yl)-methanol

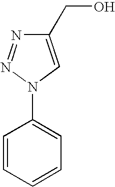

A stirred solution of 1-phenyl-1H-[1,2,3]triazole-4-carboxylic acid ethyl ester (prepared by a procedure similar to that described in Steps 1-3 of General Scheme 1) (269 mg, 1.25 mmol) in THF (6 mL) was heated to reflux. To the heated reaction mixture was added sodium borohydride (395 mg, 10.4 mmol) portionwise over a period of 15 minutes, followed by dropwise addition of MeOH (2.4 mL). Stirring was continued at reflux temperature for 1 hr. The reaction mixture was then quenched with ammonium chloride solution and the product was extracted with ethyl acetate. The organic layer was washed with saturated brine solution, dried over sodium sulfate and concentrated under reduced pressure to afford 245 mg (89%) of (1-phenyl-1H-[1,2,3]triazol-4-yl)-methanol.

### Step 2

### Synthesis of 1-Phenyl-1H-[1,2,3]triazole-4-carbaldehyde

MnO₂ (1.23 g, 14.14 mmol) was added to a stirred solution of (1-phenyl-1H-[1,2,3]triazol-4-yl)-methanol (245 mg, 1.4 mmol) in DCM (15 mL) and the resulting mixture was stirred at room temperature overnight. The mixture was filtered over a celite bed, and the filtrate was concentrated under reduced pressure to afford 271 mg (99%) of 1-phenyl-1H-[1,2,3]triazole-4-carbaldehyde.

### Step 3

### Synthesis of 1-[4-(5-Fluoro-2-trifluoromethyl-benzoyl)-piperazin-1-yl]-2-[(1-phenyl-1H-[1,2,3]triazol-4-ylmethyl)-amino]-ethanone

Sodium triacetoxy borohydride (400 mg, 1.8 mmol) was added portion wise to a stirred cold (0-5° C.) solution of 1-phenyl-1H-[1,2,3]triazole-4-carbaldehyde (243 mg, 1.42 mmol) and 2-amino-1-[4-(5-fluoro-2-trifluoromethyl-benzoyl)-piperazin-1-yl]-ethanone hydrochloride (577 mg, 1.5 mmol) (prepared according to a procedure similar to that described in Synthesis Procedure 1, using 5-fluoro-2-(trifluoromethyl)benzoic acid (Aldrich, St. Louis, Mo.) as a starting material) in DCE (15 mL) and the resulting mixture was stirred at room temperature overnight. Cold water was then added followed by saturated sodium bicarbonate solution and the product was extracted with dichloromethane. The organic layer was dried over sodium sulfate and concentrated under reduced pressure. The resulting residue was purified by preparative HPLC to afford 140 mg (20%) of 1-[4-(5-fluoro-2-trifluoromethyl-benzoyl)-piperazin-1-yl]-2-[(1-phenyl-1H-[1,2,3]triazol-4-ylmethyl)-amino]-ethanone. LCMS Purity: 100%.

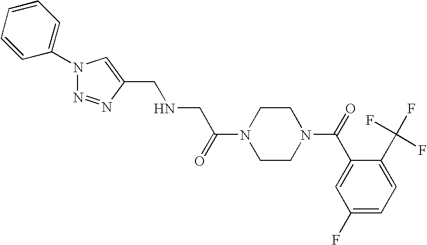

### Step 4

### Synthesis of 1-[4-(5-Fluoro-2-trifluoromethyl-benzoyl)-piperazin-1-yl]-2-[(1-phenyl-1H-[1,2,3]triazol-4-ylmethyl)-amino]-ethanone hydrochloride

A solution of 1-[4-(5-fluoro-2-trifluoromethyl-benzoyl)-piperazin-1-yl]-2-[(1-phenyl-1H-[1,2,3]triazol-4-ylmethyl)-amino]-ethanone (140 mg, 0.28 mmol) in ether. HCl (10 ml) was stirred at room temperature for 1 hr. The reaction mixture was then concentrated. The resulting residue was washed with diethyl ether followed by methanol to afford 90 mg (60%) of 1-[4-(5-fluoro-2-trifluoromethyl-benzoyl)-piperazin-1-yl]-2-[(1-phenyl-1H-[1,2,3]triazol-4-ylmethyl)-amino]-ethanone hydrochloride. LCMS Purity: 87.99%. ¹H NMR (DMSO-d₆): δ 9.6 (s, 2H), 8.94 (d, 1H), 8.0-7.84 (m, 3H), 7.7-7.5 (m, 5H), 4.4 (d, 2H), 4.28 (d, 2H), 3.86-3.4 (m, 6H), 3.24 (d, 2H).

### Example 28

### Imidazo[1,2-a]pyridine-2-carboxylic acid {2-[4-(5-fluoro-2-trifluoromethyl-benzoyl)-piperazin-1-yl]-2-oxo-ethyl}-amide

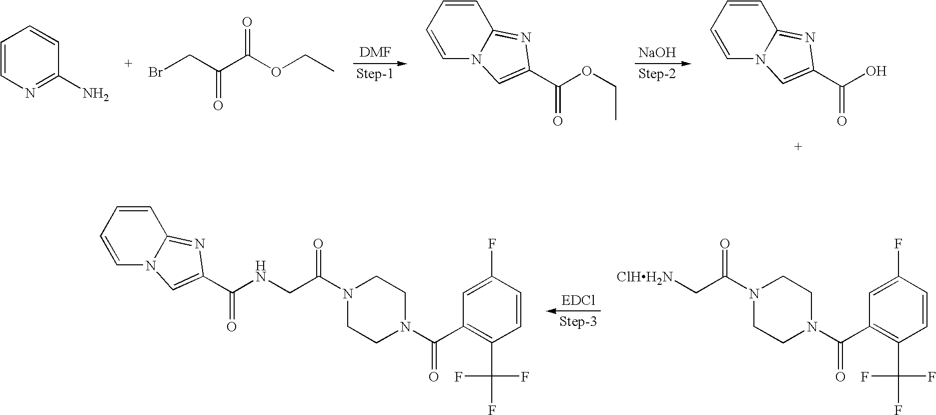

### Step 1

### Synthesis of Imidazo[1,2-a]pyridine-2-carboxylic acid ethyl ester

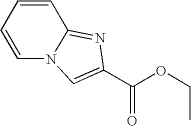

A solution of 2-aminopyridine (500 mg, 5.3 mmol) and 2-bromopyruvic acid ethyl ester (824 mg, 4.2 mmol) in DMF (5 ml) was stirred at 100° C. for 2 hours then at room temperature for 1 hr. The reaction mixture was then quenched with cold water and the product was extracted with ethyl acetate. The organic layer was dried over sodium sulfate and concentrated under reduced pressure. The resulting residue was purified by column chromatography (using 60-120 silica gel and 50% ethyl acetate in hexane as eluent) to afford 135 mg (20%) of imidazo[1,2-a]pyridine-2-carboxylic acid ethyl ester.

### Step 2

### Synthesis of Imidazo[1,2-a]pyridine-2-carboxylic acid

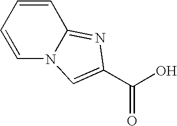

NaOH (85 mg, 2.1 mmol) was added to a stirred solution of imidazo[1,2-a]pyridine-2-carboxylic acid ethyl ester (135 mg, 0.7 mmol) in EtOH:H₂O (1:1, 5.0 mL) and the resulting mixture was heated at 60° C. for 3 hours. The reaction mixture was then concentrated under reduced pressure. Cold water was added, which was then acidified with aqueous 10% HCl solution. The resulting precipitate was isolated by filtration to afford 110 mg (73% yield) of imidazo[1,2-a]pyridine-2-carboxylic acid.

### Step 3

### Synthesis of Imidazo[1,2-a]pyridine-2-carboxylic acid {2-[4-(5-fluoro-2-trifluoromethyl-benzoyl)-piperazin-1-yl]-2-oxo-ethyl}-amide

DIPEA (220 mg, 0.3 mL, 1.7 mmol) was added to a stirred solution of imidazo[1,2-a]pyridine-2-carboxylic acid (55 mg, 0.34 mmol) in DMF (5 mL). HOBT (50 mg, 0.37 mmol) and EDCI (163 mg, 0.85 mmol) were then added at room temperature. After 2 minutes, 2-amino-1-[4-(5-fluoro-2-trifluoromethyl-benzoyl)-piperazin-1-yl]-ethanone hydrochloride (138 mg, 0.37 mmol) (prepared according to a procedure similar to that described in Synthesis Procedure 1, using 5-fluoro-2-(trifluoromethyl)benzoic acid (Aldrich, St. Louis, Mo.) as a starting material) was added and the resulting mixture was stirred at room temperature overnight. Cold water was then added and the resulting precipitate was isolated by filtration. Purification by recrystallisation from 30% ethyl acetate in hexane afforded 70 mg (43%) of imidazo[1,2-a]pyridine-2-carboxylic acid {2-[4-(5-fluoro-2-trifluoromethyl-benzoyl)-piperazin-1-yl]-2-oxo-ethyl}-amide. LCMS Purity: 90.06%. ¹H NMR (DMSO-d₆): δ 8.62 (d, 1H), 8.42 (s, 1H), 8.34 (s, 1H), 7.98 (s, 1H), 7.68-7.44 (m, 3H), 7.4 (t, 1H), 7.02 (t, 1H), 4.3 (d, 2H), 3.8-3.44 (m, 6H), 3.28 (bd, 2H).

### Example 29

### 1-Pyridin-2-yl-1H-[1,2,3]triazole-4-carboxylic acid {2-[4-(2-chloro-5-fluoro-benzoyl)-piperazin-1-yl]-2-oxo-ethyl}-amide

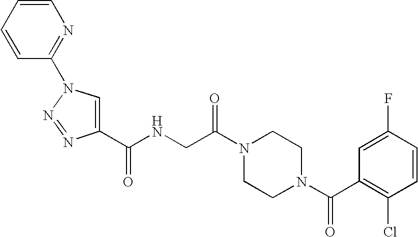

DIPEA (163 mg, 0.22 mL, 1.2 mmol) was added to a stirred solution of 1-pyridin-2-yl-1H-[1,2,3]triazole-4-carboxylic acid (prepared by a procedure similar to that described in Steps 1-3 of General Scheme 1) (60 mg, 0.31 mmol) in DMF (2 mL). HOBT (46.8 mg, 0.34 mmol) and EDCI (121 mg, 0.63 mmol) were then added at room temperature. After 2 minutes, 2-amino-1-[4-(2-chloro-5-fluoro-benzoyl)-piperazin-1-yl]-ethanone hydrochloride (106 mg, 0.31 mmol) (prepared according to a procedure similar to that described in Synthesis Procedure 1, using 2-chloro-5-fluoro benzoic acid (Aldrich, St. Louis, Mo.) as a starting material) was added and the resulting mixture was stirred at room temperature overnight. Cold water was then added and the product was extracted with ethyl acetate. The organic layer was washed with saturated brine solution then dried over sodium sulfate. The organic layer was concentrated under reduced pressure and the resulting residue was washed with a mixture of ethyl acetate and hexane to afford 66 mg (45%) of 1-pyridin-2-yl-1H-[1,2,3]triazole-4-carboxylic acid {2-[4-(2-chloro-5-fluoro-benzoyl)-piperazin-1-yl]-2-oxo-ethyl}-amide. LCMS Purity: 98.31%. ¹H NMR (DMSO-d₆): δ 9.2 (s, 1H), 8.7-8.6 (m, 2H), 8.2-8.1 (m, 2H), 7.66-7.56 (m, 2H), 7.5-7.3 (m, 2H), 4.3-4.2 (dd, 2H), 3.76-3.46 (m, 6H), 3.28-3.12 (m, 2H).

### Example 30

### 1-Pyridin-2-yl-1H-[1,2,3]triazole-4-carboxylic acid {2-oxo-2-[4-(3,4,5-trifluoro-benzoyl)-piperazin-1-yl]-ethyl}-amide

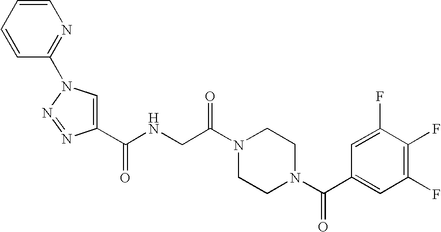

DIPEA (163 mg, 0.22 mL, 1.2 mmol) was added to a stirred solution of 1-pyridin-2-yl-1H-[1,2,3]triazole-4-carboxylic acid (prepared by a procedure similar to that described in Steps 1-3 of General Scheme 1) (60 mg, 0.31 mmol) in DMF (2 mL). HOBT (46.8 mg, 0.34 mmol) and EDCI (121 mg, 0.63 mmol) were then added at room temperature. After 2 minutes, 2-amino-1-[4-(3,4,5-trifluoro-benzoyl)-piperazin-1-yl]-ethanone hydrochloride (106 mg, 0.31 mmol) (prepared according to a procedure similar to that described in Synthesis Procedure 1, using 3,4,5-trifluoro benzoic acid (Aldrich, St. Louis, Mo.) as a starting material) was added and the resulting mixture was stirred at room temperature overnight. Cold water was then added. The resulting precipitate was isolated by filtration to afford 80 mg (54%) of 1-pyridin-2-yl-1H-[1,2,3]triazole-4-carboxylic acid {2-oxo-2-[4-(3,4,5-trifluoro-benzoyl)-piperazin-1-yl]-ethyl}-amide. LCMS Purity: 87.24%.

¹H NMR (DMSO-d₆): δ 9.2 (s, 1H), 8.7-8.6 (m, 2H), 8.2 (m, 2H), 7.66-7.6 (m, 1H), 7.55-7.45 (t, 2H), 4.3-4.2 (bs, 2H), 3.7-3.5 (bs, 6H), 3.45-3.35 (m, 2H).

### Example 31

### 1-Cyclopropyl-1H-[1,2,3]triazole-4-carboxylic acid {2-[4-(2-chloro-5-fluoro-benzoyl)-piperazin-1-yl]-2-oxo-ethyl}-amide

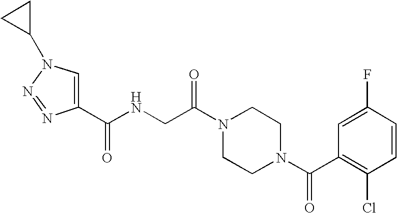

DIPEA (202 mg, 0.27 mL, 1.5 mmol) was added to a stirred solution of 1-cyclopropyl-1H-[1,2,3]triazole-4-carboxylic acid (prepared by a procedure similar to that described in Steps 1-3 of General Scheme 1) (60 mg, 0.39 mmol) in DMF (3 mL). HOBT (58.2 mg, 0.43 mmol) and EDCI (150 mg, 0.78 mmol) were then added at room temperature. After 2 minutes, 2-amino-1-[4-(2-chloro-5-fluoro-benzoyl)-piperazin-1-yl]-ethanone hydrochloride salt (131 mg, 0.39 mmol) (prepared according to a procedure similar to that described in Synthesis Procedure 1, using 2-chloro-5-fluoro benzoic acid (Aldrich, St. Louis, Mo.) as a starting material) was added and the resulting mixture was stirred at room temperature overnight. Cold water was then added. The product was extracted with ethyl acetate and the organic layer was washed with saturated brine solution, dried over sodium sulfate then concentrated under reduced pressure. The resulting residue was washed with a mixture of dichloromethane and hexane to afford 105 mg (62%) of 1-cyclopropyl-1H-[1,2,3]triazole-4-carboxylic acid {2-[4-(2-chloro-5-fluoro-benzoyl)-piperazin-1-yl]-2-oxo-ethyl}-amide. LCMS Purity: 95.07%. ¹H NMR (DMSO-d₆): δ 8.65 (s, 1H), 8.4-8.3 (t, 1H), 7.68-7.56 (m, 1H), 7.46-7.28 (m, 2H), 4.25-4.1 (dd, 2H), 4.1-4.0 (m, 1H), 3.8-3.4 (m, 7H), 3.3-3.1 (m, 2H), 1.3-1.15 (m, 2H), 1.15-1.1 (m, 2H).

### Example 32

### 1-Morpholin-4-yl-1H-[1,2,3]triazole-4-carboxylic acid {2-[4-(2-chloro-5-fluoro-benzoyl)-piperazin-1-yl]-2-oxo-ethyl}-amide

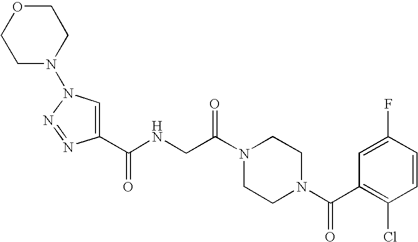

DIPEA (182 mg, 0.24 mL, 1.4 mmol) was added to a stirred solution of 1-morpholin-4-yl-1H-[1,2,3]triazole-4-carboxylic acid (prepared by a procedure similar to that described in Steps 1-3 of General Scheme 1) (70 mg, 0.35 mmol) in DMF (3 mL). HOBT (52.5 mg, 0.38 mmol) and EDCI (135 g, 0.70 mmol) were then added at room temperature. After 2 minutes, 2-amino-1-[4-(2-chloro-5-fluoro-benzoyl)-piperazin-1-yl]-ethanone hydrochloride (118.7 mg, 0.35 mmol) (prepared according to a procedure similar to that described in Synthesis Procedure 1, using 2-chloro-5-fluoro benzoic acid (Aldrich, St. Louis, Mo.) as a starting material) was added and the resulting mixture was stirred at room temperature overnight. Cold water was then added. The product was extracted with ethyl acetate and the organic layer was washed with saturated brine solution, dried over sodium sulfate then concentrated under reduced pressure. The resulting residue was washed with a mixture of dichloromethane and hexane to afford 67 mg (39%) of 1-morpholin-4-yl-1H-[1,2,3]triazole-4-carboxylic acid {2-[4-(2-chloro-5-fluoro-benzoyl)-piperazin-1-yl]-2-oxo-ethyl}-amide. ¹H NMR (DMSO-d₆): δ 8.8 (s, 1H), 8.4 (t, 1H), 7.66-7.58 (m, 1H), 7.48-7.3 (m, 2H), 4.26-4.1 (dd, 2H), 3.8 (m, 4H), 3.7 (m, 1H), 3.6 (m, 2H), 3.5 (m, 2H), 3.3 (m, 5H), 3.2 (m, 2H).

### Example 33

### N-{2-[4-(2-Chloro-5-fluoro-benzoyl)-piperazin-1-yl]-2-oxo-ethyl}-4-(2-oxo-pyrrolidin-1-yl)-benzamide

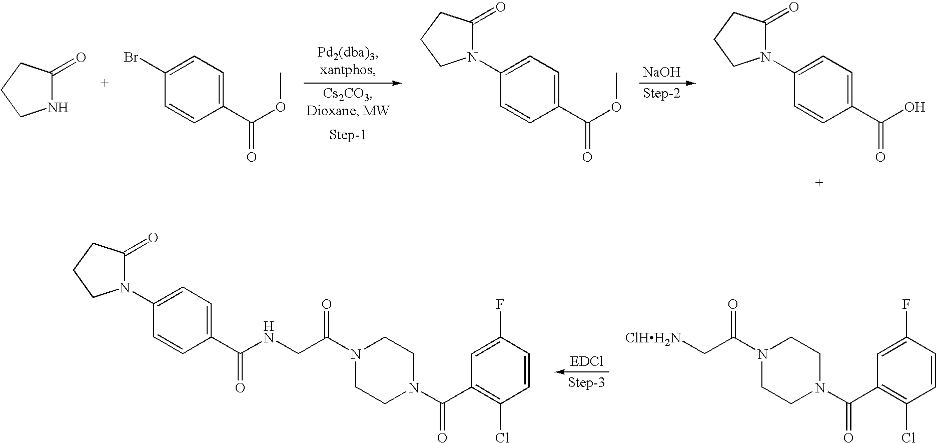

### Step 1

### Synthesis of 4-(2-Oxo-pyrrolidin-1-yl)-benzoic acid methyl ester

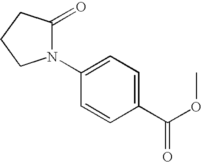

A mixture of pyrrolidine-2-one (500 mg, 5.9 mmol), 4-bromo benzoic acid methyl ester (1.5 g, 6.9 mmol), Pd₂(dba)₃ (135 mg, 0.14 mmol), xantphos (256 mg, 0.44 mmol) and cesium carbonate (2.7 g, 8.28 mmol) in dioxane (2 mL) in a seal tube closed with a teflon cap was subjected to reaction in a microwave reactor (time: 2 min, temp: 105° C., power: zero). The reaction mixture was filtered over celite and the filtrate collected was concentrated under reduced pressure. The resulting residue was diluted with cold water then the product was extracted with ethyl acetate. The organic layer was dried over sodium sulfate and concentrated under reduced pressure. The residue thus obtained was purified by column chromatography (using 60-120 silica gel and 50% ethyl acetate in hexane as eluent) to afford 485 mg (38%) of 1-phenyl-1H-imidazole-4-carboxylic acid methyl ester.

### Step 2

### Synthesis of 4-(2-Oxo-pyrrolidin-1-yl)-benzoic acid

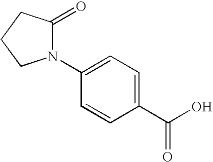

NaOH (295 mg, 7.4 mmol) was added to a stirred solution of 4-(2-oxo-pyrrolidin-1-yl)-benzoic acid methyl ester (485 mg, 0.7 mmol) in MeOH:H₂O (1:1, 5.0 mL) and stirring was continued with heating at 60° C. for 1 hr. The reaction mixture was then concentrated under reduced pressure. Cold water was then added to the residue, which was then acidified with aqueous 10% HCl solution. The resulting precipitated was isolated by filtration to afford 300 mg (66%) of 4-(2-Oxo-pyrrolidin-1-yl)-benzoic acid.

### Step 3

### Synthesis of N-{2-[4-(2-Chloro-5-fluoro-benzoyl)-piperazin-1-yl]-2-oxo-ethyl}-4-(2-oxo-pyrrolidin-1-yl)-benzamide

DIPEA (200 mg, 0.27 mL, 1.5 mmol) was added to a stirred solution of 4-(2-oxo-pyrrolidin-1-yl)-benzoic acid (70 mg, 0.34 mmol) in DMF (5 mL). HOBT (50 mg, 0.37 mmol) and EDCI (163 mg, 0.85 mmol) were then added at room temperature. After 2 minutes, 2-amino-1-[4-(2-chloro-5-fluoro-benzoyl)-piperazin-1-yl]-ethanone hydrochloride (126 mg, 0.37 mmol) (prepared according to a procedure similar to that described in Synthesis Procedure 1, using 2-chloro-5-fluoro benzoic acid (Aldrich, St. Louis, Mo.) as a starting material) was added and the resulting mixture was stirred at room temperature overnight. Cold water was then added. The resulting precipitate was isolated by filtration and purified by recrystallisation from 20% ethyl acetate in hexane to afford 101 mg (61%) of N-{2-[4-(2-chloro-5-fluoro-benzoyl)-piperazin-1-yl]-2-oxo-ethyl}-4-(2-oxo-pyrrolidin-1-yl)-benzamide. ¹H NMR (DMSO-d₆): δ 8.7 (t, 1H), 7.92 (d, 2H), 7.82 (d, 2H), 7.64 (bs, 1H), 7.48 (d, 2H), 4.2 (d, 2H), 3.9 (t, 2H), 3.8 (d, 4H), 3.6 (bs, 2H), 3.3 (d, 2H), 2.5 (m, 2H), 2.1 (m, 2H).

### Example 34

### 1-Cyclopentyl-1H-[1,2,3]triazole-4-carboxylic acid {2-oxo-2-[4-(3,4,5-trifluoro-benzoyl)-piperazin-1-yl]-ethyl}-amide

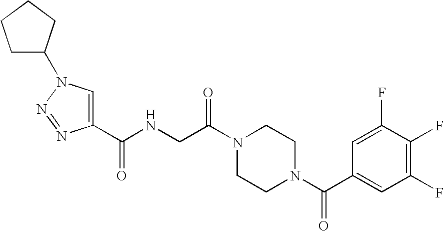

DIPEA (171 mg, 0.23 mL, 1.3 mmol) was added to a stirred solution of 1-cyclopentyl-1H-[1,2,3]triazole-4-carboxylic acid (prepared by a procedure similar to that described in Steps 1-3 of General Scheme 1) (60 mg, 0.33 mmol) in DMF (3 mL). HOBT (49.2 mg, 0.36 mmol) and EDCI (127 mg, 0.66 mmol) were then added at room temperature. After 2 minutes, 2-amino-1-[4-(3,4,5-trifluoro-benzoyl)-piperazin-1-yl]-ethanone hydrochloride salt (111.8 mg, 0.33 mmol) (prepared according to a procedure similar to that described in Synthesis Procedure 1, using 3,4,5-trifluoro benzoic acid (Aldrich, St. Louis, Mo.) as a starting material) was added and the resulting mixture was stirred at room temperature overnight. Cold water was then added. The resulting precipitate was isolated by filtration to afford 110 mg (73%) of 1-cyclopentyl-1H-[1,2,3]triazole-4-carboxylic acid {2-oxo-2-[4-(3,4,5-trifluoro-benzoyl)-piperazin-1-yl]-ethyl}-amide. ¹H NMR (DMSO-d₆): δ 8.65 (s, 1H), 8.35 (t, 1H), 7.5 (t, 2H), 5.0 (m, 1H), 4.2 (s, 2H), 3.7-3.4 (m, 6H), 2.3-2.1 (m, 2H), 2.1-1.9 (m, 2H), 1.85-1.75 (m, 2H), 1.75-1.6 (m, 2H).

### Example 35

### 1-Cyclopentyl-1H-[1,2,3]triazole-4-carboxylic acid {2-[4-(2-chloro-5-fluoro-benzoyl)-piperazin-1-yl]-2-oxo-ethyl}-amide

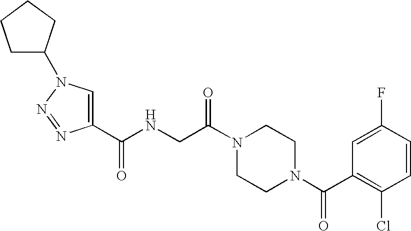

DIPEA (171 mg, 0.23 mL, 1.3 mmol) was added to a stirred solution of 1-cyclopentyl-1H-[1,2,3]triazole-4-carboxylic acid (prepared by a procedure similar to that described in Steps 1-3 of General Scheme 1) (60 mg, 0.33 mmol) in DMF (3 mL). HOBT (49.2 mg, 0.36 mmol) and EDCI (127 mg, 0.66 mmol) were then added at room temperature. After 2 minutes, 2-amino-1-[4-(2-chloro-5-fluoro-benzoyl)-piperazin-1-yl]-ethanone hydrochloride (111.3 mg, 0.33 mmol) (prepared according to a procedure similar to that described in Synthesis Procedure 1, using 2-chloro-5-fluoro benzoic acid (Aldrich, St. Louis, Mo.) as a starting material) was added and the resulting mixture was stirred at room temperature overnight. Cold water was then added. The resulting precipitate was isolated by filtration to afford 103 mg (67%) of 1-cyclopentyl-1H-[1,2,3]triazole-4-carboxylic acid {2-[4-(2-chloro-5-fluoro-benzoyl)-piperazin-1-yl]-2-oxo-ethyl}-amide. LCMS-purity: ¹H NMR (DMSO-d₆): δ 8.65 (s, 1H), 8.3 (t, 1H), 7.65-7.55 (m, 1H), 7.45-7.3 (m, 2H), 5.0 (q, 1H), 4.3-4.1 (dd, 2H), 3.8-3.55 (m, 4H), 3.55-3.45 (m, 2H), 3.3-3.1 (m, 2H), 2.3-2.1 (m, 2H), 2.1-1.9 (m, 2H), 1.9-1.4 (m, 4H).

### Example 36

### 5-Pyridin-3-yl-1H-pyrazole-3-carboxylic acid {2-[4-(5-fluoro-2-trifluoromethyl-benzoyl)-piperazin-1-yl]-2-oxo-ethyl}-amide

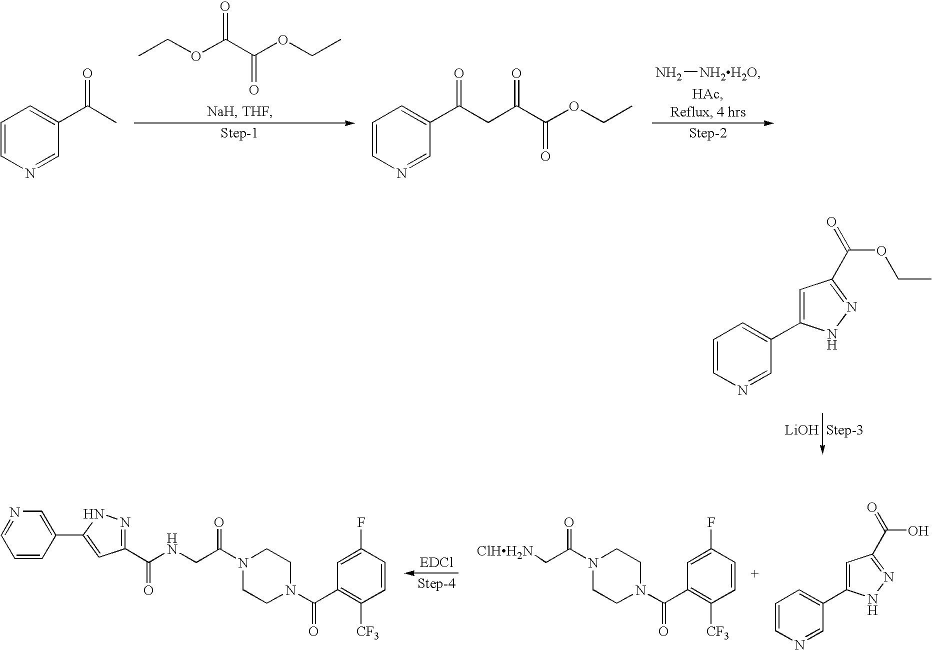

### Step 1

### Synthesis of 2,4-Dioxo-4-pyridin-3-yl-butyric acid ethyl ester

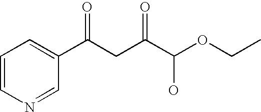

To a stirred solution of NaH (60% w/w dispersion in oil) (991 mg, 24.8 mmol) in THF (25 mL), was added diethyl oxalate (2.41 g, 16.52 mmol) and the resulting mixture was heated to reflux. At reflux temperature, 1-pyridin-3-yl-ethanone (1.0 g, 8.26 mmol) was added and stirring was continued at reflux temperature for a further 15 minutes. The reaction mixture was then quenched with ice cold water, acidified with aqueous 2.4N HCl solution and extracted with ethyl acetate. The organic layer was dried over sodium sulfate and concentrated under reduced pressure to afford 1.62 g (89%) of 2,4-dioxo-4-pyridin-3-yl-butyric acid ethyl ester.

### Step 2

### Synthesis of 5-Pyridin-3-yl-1H-pyrazole-3-carboxylic acid ethyl ester

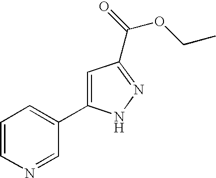

Hydrazine hydrate (398 mg, 7.96 mmol) was added to a solution of 2,4-dioxo-4-pyridin-3-yl-butyric acid ethyl ester (1.6 g, 7.23 mmol) in ethanol (40 mL) and stirring was continued at reflux temperature for 4 hrs. The reaction mixture was then concentrated under reduced pressure. The residue was dissolved in chloroform, washed with water and the organics were dried over sodium sulfate then concentrated under reduced pressure. The residue was washed with ethyl acetate to afford 541 mg (34%) of 5-pyridin-3-yl-1H-pyrazole-3-carboxylic acid ethyl ester.

### Step 3

### Synthesis of 5-Pyridin-3-yl-1H-pyrazole-3-carboxylic acid

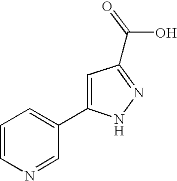

To a stirred solution of 5-pyridin-3-yl-1H-pyrazole-3-carboxylic acid ethyl ester (540 mg, 2.48 mmol) in a mixture of THF (20 mL), methanol (12 mL) and H₂O (6 mL) was added, LiOH.H₂O (313 mg, 7.4 mmol) and the resulting mixture was stirred at 50° C. overnight. The mixture was then concentrated under reduced pressure. The residue thus obtained was diluted with water, acidified with concentrated HCl solution and extracted with ethyl acetate. The organic layer was dried over sodium sulphate and concentrated under reduced pressure. The resulting residue was purified by washing with diethyl ether followed by methanol to afford 512 mg (91%) of 5-pyridin-3-yl-1H-pyrazole-3-carboxylic acid.

### Step 4

### Synthesis of 5-Pyridin-3-yl-1H-pyrazole-3-carboxylic acid {2-[4-(5-fluoro-2-trifluoromethyl-benzoyl)-piperazin-1-yl]-2-oxo-ethyl}-amide

DIPEA (291 mg, 0.4 mL, 2.25 mmol) was added drop wise to 5-pyridin-3-yl-1H-pyrazole-3-carboxylic acid (100 mg, 0.44 mmol) in DMF (2 mL). HOBT (63 mg, 0.46 mmol) and EDCI (90 mg, 0.46 mmol) were then added. After 2 minutes, 2-amino-1-[4-(5-fluoro-2-trifluoromethyl-benzoyl)-piperazin-1-yl]-ethanone hydrochloride (163.8 mg, 0.44 mmol) (prepared according to a procedure similar to that described in Synthesis Procedure 1, using 5-fluoro-2-(trifluoromethyl)benzoic acid (Aldrich, St. Louis, Mo.) as a starting material) was added. The resulting mixture was stirred at room temperature overnight. Cold water was then added and the product was extracted with ethyl acetate. The organic layer was dried over sodium sulphate and concentrated under reduced pressure. The residue thus obtained was purified by recrystallisation from ethyl acetate to afford 117 mg (52%) of 5-pyridin-3-yl-1H-pyrazole-3-carboxylic acid {2-[4-(5-fluoro-2-trifluoromethyl-benzoyl)-piperazin-1-yl]-2-oxo-ethyl}-amide. LCMS Purity: 96.24%. ¹H NMR (DMSO-d₆): δ 13.8 (s, 1H), 9.04 (s, 1H), 8.54 (s, 1H), 8.1 (m, 2H), 7.9 (m, 1H), 7.5 (m, 3H), 7.2 (m, 1H), 4.1 (m, 2H), 3.4 (m, 6H), 3.1 (d, 3H).

### Example 37

### 9H-Carbazole-3-carboxylic acid {2-[4-(5-fluoro-2-trifluoromethyl-benzoyl)-piperazin-1-yl]-2-oxo-ethyl}-amide

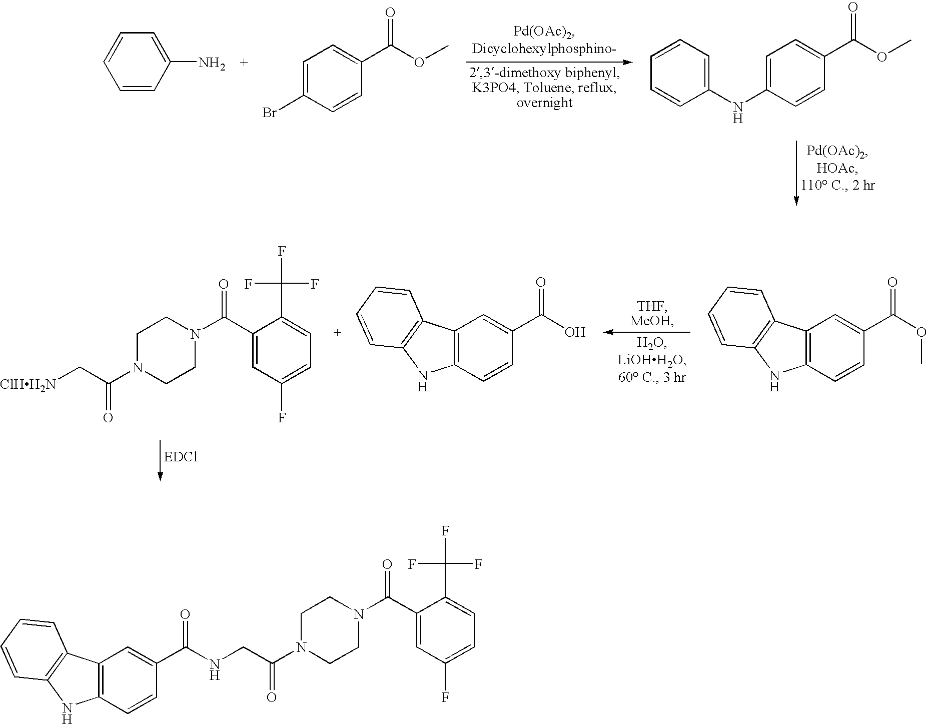

### Step 1

### Synthesis of 4-Phenylamino-benzoic acid methyl ester

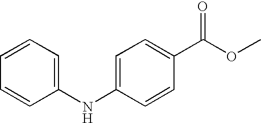

A mixture of Pd(OAc)₂ (5 mg, 0.02 mmol), dicyclohexylphosphino-2′,3′-dimethoxy biphenyl (17 mg, 0.04 mmol) in toluene (20 mL) was purged with argon gas for 10 minutes, To the resulting mixture was added, aniline (136 mg, 1.46 mmol) and K₃PO₄(136 mg, 1.46 mmol) and purged with argon gas for 10 minutes and to which added 4-bromobenzoicacid methyl ester (300 mg, 1.4 mmol) and heated to reflux overnight. The reaction mixture was diluted with ethyl acetate and filtered the residue; the filtrate collected was concentrated under reduced pressure to afford the crude mass. The resultant mass was purified by column chromatography (using 60-120 silica gel and 10% EtOAc in Hexane as eluent) to afford 201 mg (63.4%) of 4-Phenylamino-benzoic acid methyl ester.

### Step 2

### Synthesis of 9H-Carbazole-3-carboxylic acid methyl ester

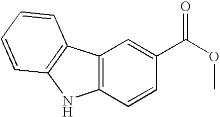

A mixture of 4-Phenylamino-benzoic acid methyl ester (103 mg, 0.45 mmol), Pd(OAc)₂ (203 mg, 0.9 mmol), in HOAc (2.5 mL) was heated at 110° C. for 2 hr. The reaction mixture was quenched with cold water, extracted with ethyl acetate, dried over sodium sulfate and concentrated under reduced pressure to afford the residue. The residue obtained was purified by column chromatography (using 60-120 silica gel and 10% EtOAc in Hexane as eluent) to afford 120 mg (60.3%) of 9H-Carbazole-3-carboxylic acid methyl ester.

### Step 3

### Synthesis of 9H-Carbazole-3-carboxylic acid

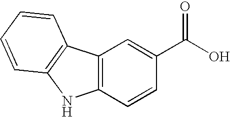

LiOH.H₂O (680 mg, 16.2 mmol) was added to a stirred solution of 9H-Carbazole-3-carboxylic acid methyl ester (120 mg, 0.5 mmol) in THF:H₂O:MeOH (3:1:1, 5 mL), and the resulting mixture was stirred at 60° C. for 3 hr. The reaction mixture was concentrated under reduced pressure to get the residue. Cold water was then added and acidified it with 10% aqueous HCl, filtered the solid precipitated to afford 104 mg (92.85% yield) of 9H-Carbazole-3-carboxylic acid.

### Step 4

### Synthesis of 9H-Carbazole-3-carboxylic acid {2-[4-(5-fluoro-2-trifluoromethyl-benzoyl)-piperazin-1-yl]-2-oxo-ethyl}-amide

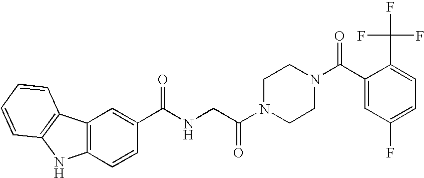

DIPEA (137 mg, 0.18 mL, 1.05 mmol) followed by HOBT (35 mg, 0.26 mmol) and EDCI (113 mg, 0.59 mmol) were added to a stirred solution of 9H-Carbazole-3-carboxylic acid (50 mg, 0.23 mmol) in DMF (5 mL) at room temperature. After 2 minutes 2-Amino-1-[4-(5-fluoro-2-trifluoromethyl-benzoyl)-piperazin-1-yl]-ethanone hydrochloride salt (prepared according to a procedure similar to that described in synthesis procedure 1, using 5-Fluoro-2-trifluoromethyl-benzoic acid (Aldrich, St. Louis, Mo.) as a starting material) (96 mg, 0.26 mmol) was added and the resulting mixture was stirred at room temperature overnight. Cold water was then added, filtered the solid precipitated. The solid obtained was purified by recrystallisation from water to afford 83 mg (66.93%) of 9H-Carbazole-3-carboxylic acid {2-[4-(5-fluoro-2-trifluoromethyl-benzoyl)-piperazin-1-yl]-2-oxo-ethyl}-amide. LCMS Purity: 91.51%. ¹H NMR (DMSO-d₆): δ 11.6 (s, 1H), 8.8 (s, 1H), 8.2 (d, 1H), 8.0 (d, 2H), 7.6 (m, 3H), 7.4 (t, 1H), 7.2 (t, 1H), 4.2 (d, 2H), 4.0-3.4 (m, 6H), 3.3 (d, 2H).

### Example 38

### 5-(3-Hydroxy-phenyl)-isoxazole-3-carboxylic acid {2-[4-(5-fluoro-2-trifluoromethyl-benzoyl)-piperazin-1-yl]-2-oxo-ethyl}-amide

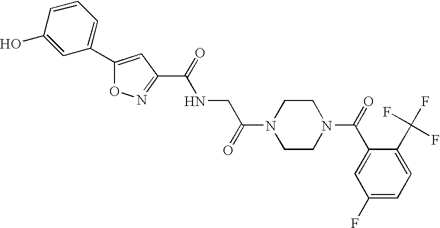

DIPEA (153 mg, 0.2 mL, 1.18 mmol) followed by HOBT (48 mg, 0.35 mmol) and EDCI (69 mg, 0.35 mmol) were added to a stirred solution of 5-(3-Hydroxy-phenyl)-isoxazole-3-carboxylic acid (69.3 mg, 0.34 mmol) (prepared according to a procedure similar to that described in synthesis procedure 3, steps 1-4-b, using 1-(3-hydroxyphenyl)ethanone (Aldrich, St. Louis, Mo.) as starting material) in DMF (2 mL) at room temperature. After 2 minutes 2-Amino-1-[4-(5-fluoro-2-trifluoromethyl-benzoyl)-piperazin-1-yl]-ethanone hydrochloride salt (prepared according to a procedure similar to that described in synthesis procedure 1, using 5-Fluoro-2-trifluoromethyl-benzoic acid (Aldrich, St. Louis, Mo.) as a starting material) (125 mg, 0.34 mmol) was added and the resulting mixture was stirred at room temperature overnight. Cold water was then added and extracted with ethyl acetate. The organic layer was washed with brine and dried over Na₂SO₄, concentrated under reduced pressure to afford the residue. The residue obtained was purified by recrystallisation from 10% EtOAc in Hexane to afford 48 mg (27.5%) of 5-(3-Hydroxy-phenyl)-isoxazole-3-carboxylic acid {2-[4-(5-fluoro-2-trifluoromethyl-benzoyl)-piperazin-1-yl]-2-oxo-ethyl}-amide. LCMS Purity: 96.77%. ¹H NMR (DMSO-d₆): δ 9.9 (s, 1H), 8.7 (t, 1H), 7.9 (t, 1H), 7.6 (m, 2H), 7.3 (m, 4H), 6.9 (m, 1H), 4.1 (m, 2H), 3.4 (m, 6H), 3.0 (m, 2H).

### Example 39

### 5-(4-Hydroxy-phenyl)-isoxazole-3-carboxylic acid {2-[4-(5-fluoro-2-trifluoromethyl-benzoyl)-piperazin-1-yl]-2-oxo-ethyl}-amide

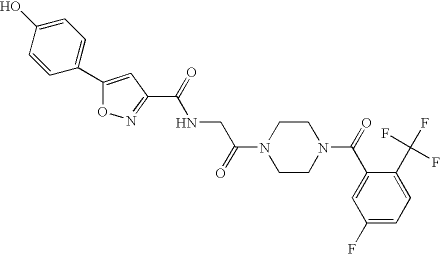

DIPEA (153 mg, 0.2 mL, 1.18 mmol) followed by HOBT (48 mg, 0.35 mmol) and EDCI (69 mg, 0.35 mmol) were added to a stirred solution of 5-(4-Hydroxy-phenyl)-isoxazole-3-carboxylic acid (69.3 mg, 0.34 mmol) (prepared according to a procedure similar to that described in synthesis procedure 3, steps 1-4-b, using 1-(4-hydroxyphenyl)ethanone (Aldrich, St. Louis, Mo.) as starting material) in DMF (2 mL) at room temperature. After 2 minutes 2-Amino-1-[4-(5-fluoro-2-trifluoromethyl-benzoyl)-piperazin-1-yl]-ethanone hydrochloride salt (prepared according to a procedure similar to that described in synthesis procedure 1, using 5-Fluoro-2-trifluoromethyl-benzoic acid (Aldrich, St. Louis, Mo.) as a starting material) (125 mg, 0.34 mmol) was added and the resulting mixture was stirred at room temperature overnight. Cold water was then added and extracted with ethyl acetate. The organic layer was washed with brine and dried over Na₂SO₄, concentrated under reduced pressure to afford the residue. The residue obtained was purified by stirring in ethyl acetate at room temperature for 10 minutes and then filtered to afford 101 mg (57.3%) of 5-(4-Hydroxy-phenyl)-isoxazole-3-carboxylic acid {2-[4-(5-fluoro-2-trifluoromethyl-benzoyl)-piperazin-1-yl]-2-oxo-ethyl}-amide. LCMS Purity: 93.08%. ¹H NMR (DMSO-d₆): δ 10.2 (s, 1H), 8.6 (t, 1H), 7.9 (m, 1H), 7.76 (d, 2H), 7.5 (m, 2H), 7.16 (s, 1H), 6.9 (d, 2H), 4.1 (m, 2H), 3.4 (m, 6H), 3.0 (m, 2H).

### Example 40

### 5-(3-Fluoro-phenyl)-isoxazole-3-carboxylic acid {2-[4-(5-fluoro-2-trifluoromethyl-benzoyl)-piperazin-1-yl]-2-oxo-ethyl}-amide

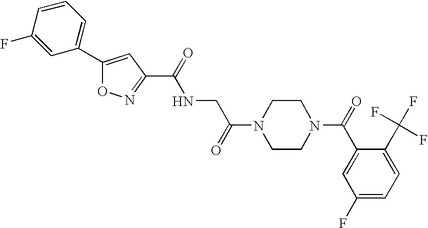

DIPEA (153 mg, 0.2 mL, 1.18 mmol) followed by HOBT (48 mg, 0.35 mmol) and EDCI (69 mg, 0.35 mmol) were added to a stirred solution of 5-(3-Fluoro-phenyl)-isoxazole-3-carboxylic acid (69.96 mg, 0.34 mmol) (prepared according to a procedure similar to that described in synthesis procedure 3, steps 1-4-b, using 3′-Fluoro-acetophenone (Aldrich, St. Louis, Mo.) as starting material) in DMF (2 mL) at room temperature. After 2 minutes 2-Amino-1-[4-(5-fluoro-2-trifluoromethyl-benzoyl)-piperazin-1-yl]-ethanone hydrochloride salt (prepared according to a procedure similar to that described in synthesis procedure 1, using 5-Fluoro-2-trifluoromethyl-benzoic acid (Aldrich, St. Louis, Mo.) as a starting material) (125 mg, 0.34 mmol) was added and the resulting mixture was stirred at room temperature overnight. Cold water was then added and extracted with ethyl acetate. The organic layer was washed with brine and dried over Na₂SO₄, concentrated under reduced pressure to afford the residue. The residue obtained was purified by recrystallisation from 10% EtOAc in Hexane to afford 45 mg (57.3%) of 5-(3-Fluoro-phenyl)-isoxazole-3-carboxylic acid {2-[4-(5-fluoro-2-trifluoromethyl-benzoyl)-piperazin-1-yl]-2-oxo-ethyl}-amide. LCMS Purity: 92.46%. ¹H NMR (DMSO-d₆): δ 8.72 (m, 1H), 7.86 (m, 1H), 7.78 (m, 2H), 7.44 (m, 4H), 7.38 (m, 1H), 4.1 (m, 2H), 3.4 (m, 6H), 3.0 (m, 2H).

### Example 41

### 5-Pyridin-2-yl-1H-pyrazole-3-carboxylic acid {2-[4-(5-fluoro-2-trifluoromethyl-benzoyl)-piperazin-1-yl]-2-oxo-ethyl}-amide

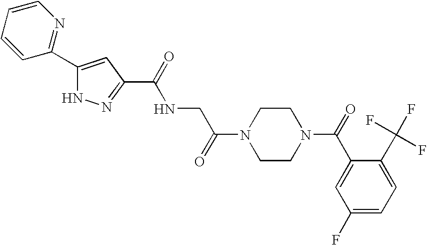

DIPEA (315 mg, 0.42 mL, 2.44 mmol) followed by HOBT (70 mg, 0.52 mmol) and EDCI (98.3 mg, 0.52 mmol) were added to a stirred solution of 5-Pyridin-2-yl-1H-pyrazole-3-carboxylic acid (110 mg, 0.48 mmol) (prepared according to a procedure similar to that described in Example-36, steps 1-3, using 1-pyridin-2-yl-ethanone (Aldrich, St. Louis, Mo.) as starting material) in DMF (2.5 mL) at room temperature. After 2 minutes 2-Amino-1-[4-(5-fluoro-2-trifluoromethyl-benzoyl)-piperazin-1-yl]-ethanone hydrochloride salt (prepared according to a procedure similar to that described in synthesis procedure 1, using 5-Fluoro-2-trifluoromethyl-benzoic acid (Aldrich, St. Louis, Mo.) as a starting material) (180 mg, 0.48 mmol) was added and the resulting mixture was stirred at room temperature overnight. Cold water was then added and extracted with ethyl acetate. The organic layer was washed with brine and dried over Na₂SO₄, concentrated under reduced pressure to afford the residue. The residue obtained was purified by recrystallisation from methanol to afford 30 mg (12.3%) of 5-Pyridin-2-yl-1H-pyrazole-3-carboxylic acid {2-[4-(5-fluoro-2-trifluoromethyl-benzoyl)-piperazin-1-yl]-2-oxo-ethyl}-amide. LCMS Purity: 93.66%. ¹H NMR (DMSO-d₆): δ 14.0 (s, 1H), 8.7 (m, 1H), 8.6 (m, 1H), 8.06 (m, 1H), 7.9 (m, 3H), 7.5 (m, 2H), 7.1 (m, 2H), 4.1 (m, 2H), 3.4 (m, 6H), 3.1 (m, 2H).

### Example 42

### 1-(3-Fluoro-phenyl)-1H-[1,2,3]triazole-4-carboxylic acid {2-[4-(5-fluoro-2-trifluoromethyl-benzoyl)-piperazin-1-yl]-2-oxo-ethyl}-amide

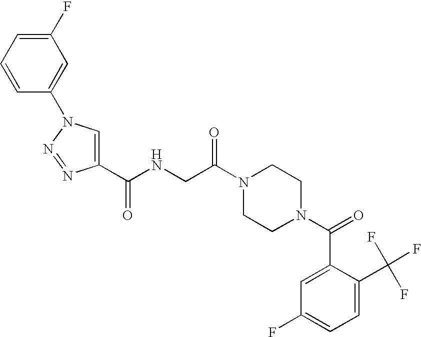

DIPEA (168 mg, 0.22 mL, 1.3 mmol) followed by HOBT (43 mg, 0.32 mmol) and EDCI (139 mg, 0.72 mmol) were added to a stirred solution of 1-(3-Fluoro-phenyl)-1H-[1,2,3]triazole-4-carboxylic acid (60 mg, 0.29 mmol) (prepared according to a procedure similar to that described in steps 1-3 of General scheme-A from 3-Fluoro aniline) in DMF (5 mL) at room temperature. After 2 minutes 2-Amino-1-[4-(5-fluoro-2-trifluoromethyl-benzoyl)-piperazin-1-yl]-ethanone hydrochloride salt (prepared according to a procedure similar to that described in synthesis procedure 1, using 5-Fluoro-2-trifluoromethyl-benzoic acid (Aldrich, St. Louis, Mo.) as a starting material) (118 mg, 0.32 mmol) was added and the resulting mixture was stirred at room temperature overnight. Cold water was then added and filtered the solid precipitated, dried to afford 110 mg (72.8%) of 1-(3-Fluoro-phenyl)-1H-[1,2,3]triazole-4-carboxylic acid {2-[4-(5-fluoro-2-trifluoromethyl-benzoyl)-piperazin-1-yl]-2-oxo-ethyl}-amide. LCMS Purity: 94.83%. ¹H NMR (DMSO-d₆): δ 9.44 (s, 1H), 8.6 (t, 1H), 8.0 (t, 3H), 7.74 (q, 1H), 7.64 (m, 2H), 7.46 (t, 1H), 4.3 (d, 2H), 3.8-3.4 (m, 6H), 3.1 (d, 2H).

### Example 43

### 1-(3-Fluoro-phenyl)-1H-[1,2,3]triazole-4-carboxylic acid {2-oxo-2-[4-(3,4,5-trifluoro-benzoyl)-piperazin-1-yl]-ethyl}-amide

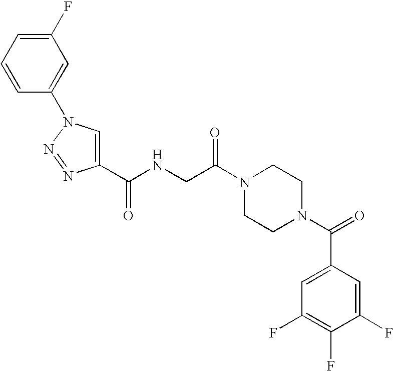

DIPEA (168 mg, 0.22 mL, 1.3 mmol) followed by HOBT (43 mg, 0.32 mmol) and EDCI (139 mg, 0.72 mmol) were added to a stirred solution of 1-(3-Fluoro-phenyl)-1H-[1,2,3]triazole-4-carboxylic acid (60 mg, 0.29 mmol) (prepared according to a procedure similar to that described in steps 1-3 of General scheme-A from 3-Fluoro aniline) in DMF (5 mL) at room temperature. After 2 minutes 2-Amino-1-[4-(3,4,5-trifluoro-benzoyl)-piperazin-1-yl]ethanone hydrochloride salt (prepared according to a procedure similar to that described in synthesis procedure 1, using 3,4,5-trifluoro-benzoic acid (Aldrich, St. Louis, Mo.) as a starting material) (107 mg, 0.32 mmol) was added and the resulting mixture was stirred at room temperature overnight. Cold water was then added and filtered the solid precipitated, dried to afford 121 mg (85.2%) of 1-(3-Fluoro-phenyl)-1H-[1,2,3]triazole-4-carboxylic acid {2-oxo-2-[4-(3,4,5-trifluoro-benzoyl)-piperazin-1-yl]-ethyl}-amide. LCMS Purity: 93.74%. ¹H NMR (DMSO-d₆): δ 9.44 (s, 1H), 8.6 (t, 1H), 8.0 (t, 2H), 7.74 (q, 1H), 7.56-7.34 (m, 2H), 4.3 (b, 2H), 3.76-3.48 (m, 6H), 3.4 (b, 2H).

### Example 44

### 1-(3-Fluoro-phenyl)-1H-[1,2,3]triazole-4-carboxylic acid {2-[4-(2-chloro-5-fluoro-benzoyl)-piperazin-1-yl]-2-oxo-ethyl}-amide

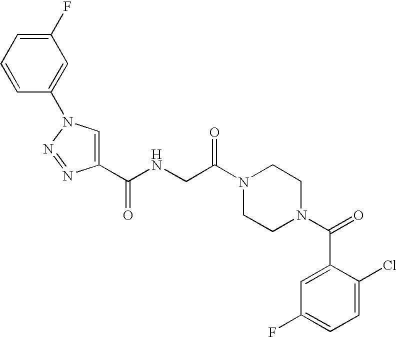

DIPEA (168 mg, 0.22 mL, 1.3 mmol) followed by HOBT (43 mg, 0.32 mmol) and EDCI (139 mg, 0.72 mmol) were added to a stirred solution of 1-(3-Fluoro-phenyl)-1H-[1,2,3]triazole-4-carboxylic acid (60 mg, 0.29 mmol) (prepared according to a procedure similar to that described in steps 1-3 of General scheme-A from 3-Fluoro aniline) in DMF (5 mL) at room temperature. After 2 minutes 2-Amino-1-[4-(2-Chloro-5-fluoro-benzoyl)-piperazin-1-yl]-ethanone hydrochloride salt (prepared according to a procedure similar to that described in synthesis procedure 1, using 2-Chloro-5-fluoro-benzoic acid (Aldrich, St. Louis, Mo.) as a starting material) (107 mg, 0.32 mmol) was added and the resulting mixture was stirred at room temperature overnight. Cold water was then added and filtered the solid precipitated, dried to afford 120 mg (85%) of 1-(3-Fluoro-phenyl)-1H-[1,2,3]triazole-4-carboxylic acid {2-[4-(2-chloro-5-fluoro-benzoyl)-piperazin-1-yl]-2-oxo-ethyl}-amide. LCMS Purity: 95.29%. ¹H NMR (DMSO-d₆): δ 9.49 (s, 1H), 8.6 (t, 1H), 8.0 (t, 2H), 7.74-7.56 (m, 2H), 7.48-7.3 (m, 2H), 4.1 (d, 2H), 3.8-3.44 (m, 6H), 3.26 (d, 2H).

### Example 45

### 1-(2-Fluoro-phenyl)-1H-[1,2,3]triazole-4-carboxylic acid {2-[4-(2-chloro-5-fluoro-benzoyl)-piperazin-1-yl]-2-oxo-ethyl}-amide

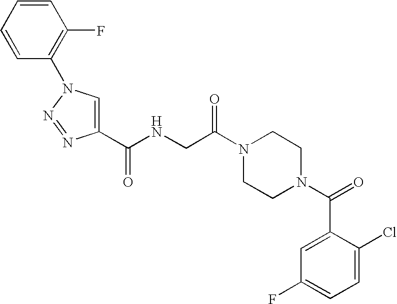

DIPEA (162 mg, 0.22 mL, 1.25 mmol) followed by HOBT (46.6 mg, 0.34 mmol) and EDCI (120 mg, 0.62 mmol) were added to a stirred solution of 1-(2-Fluoro-phenyl)-1H-[1,2,3]triazole-4-carboxylic acid (65 mg, 0.3 mmol) (prepared according to a procedure similar to that described in steps 1-3 of General scheme-A from 2-Fluoro aniline) in DMF (3 mL) at room temperature. After 2 minutes 2-Amino-1-[4-(2-Chloro-5-fluoro-benzoyl)-piperazin-1-yl]-ethanone hydrochloride salt (prepared according to a procedure similar to that described in synthesis procedure 1, using 2-Chloro-5-fluoro-benzoic acid (Aldrich, St. Louis, Mo.) as a starting material) (105.4 mg, 0.31 mmol) was added and the resulting mixture was stirred at room temperature overnight. Cold water was then added and filtered the solid precipitated. The solid obtained was purified by recrystallisation from a mixture of EtOAc and hexane to afford 71 mg (46.4%) of 1-(2-Fluoro-phenyl)-1H-[1,2,3]triazole-4-carboxylic acid {2-[4-(2-chloro-5-fluoro-benzoyl)-piperazin-1-yl]-2-oxo-ethyl}-amide. LCMS Purity: 97.16%. ¹H NMR (DMSO-d₆): δ 9.1 (s, 1H), 8.6 (t, 1H), 7.9 (t, 1H), 7.7-7.54 (m, 3H), 7.52-7.4 (m, 3H), 4.3-4.1 (d, 2H), 3.8-3.55 (m, 4H), 3.5 (s, 2H), 3.28-3.14 (m, 2H).

### Example 46

### 1-(2-Fluoro-phenyl)-1H-[1,2,3]triazole-4-carboxylic acid {2-[4-(2-chloro-5-fluoro-benzoyl)-piperazin-1-yl]-2-oxo-ethyl}-amide

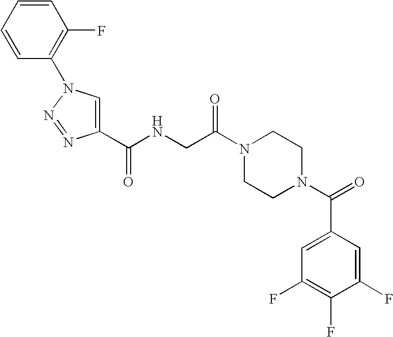

DIPEA (162 mg, 0.22 mL, 1.25 mmol) followed by HOBT (46.6 mg, 0.34 mmol) and EDCI (120 mg, 0.62 mmol) were added to a stirred solution of 1-(2-Fluoro-phenyl)-1H-[1,2,3]triazole-4-carboxylic acid (65 mg, 0.3 mmol) (prepared according to a procedure similar to that described in steps 1-3 of General scheme-A from 2-Fluoro aniline) in DMF (3 mL) at room temperature. After 2 minutes 2-Amino-1-[4-(3,4,5-trifluoro-benzoyl)-piperazin-1-yl]ethanone hydrochloride salt (prepared according to a procedure similar to that described in synthesis procedure 1, using 3,4,5-trifluoro-benzoic acid (Aldrich, St. Louis, Mo.) as a starting material) (105.9 mg, 0.31 mmol) was added and the resulting mixture was stirred at room temperature overnight. Cold water was then added and filtered the solid precipitated, washed with hexane to afford 110 mg (71.5%) of 1-(2-Fluoro-phenyl)-1H-[1,2,3]triazole-4-carboxylic acid {2-[4-(2-chloro-5-fluoro-benzoyl)-piperazin-1-yl]-2-oxo-ethyl}-amide. LCMS Purity: 96.2%. ¹H NMR (DMSO-d₆): δ 9.1 (s, 1H), 8.6-8.5 (t, 1H), 7.9-7.8 (t, 1H), 7.7-7.54 (m, 2H), 7.52-7.4 (q, 3H), 4.2 (s, 2H), 3.75-3.4 (m, 8H).

### Example 47

### 4-(2-Fluoro-phenyl)-pyrazole-1-carboxylic acid {2-oxo-2-[4-(4-trifluoromethyl-pyridine-3-carbonyl)-piperazin-1-yl]-ethyl}-amide

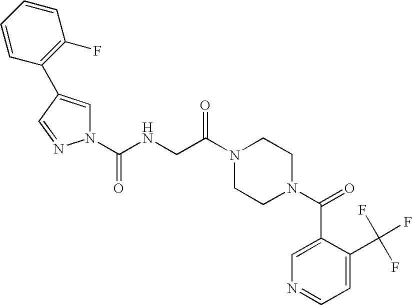

A mixture of 4-(2-Fluoro-phenyl)-1H-pyrazole (prepared according to synthesis procedure 4, as example 15)(100 mg, 0.5 mmol), DIPEA (143 mg, 0.19 ml, 1.1 mmol) and DCM (3.5 ml) was added to a stirred solution of triphosgene (60 mg, 0.2 mmol) in DCM (8 mL) at room temperature. After 30 minutes, to the above solution, a mixture of 2-amino-1-[4-(4-trifluoromethyl-pyridine-3-carbonyl)-piperazin-1-yl]-ethanone hydrochloride salt (prepared by the similar method described in Synthesis procedure 1 using 4-trifluoromethyl nicotinic acid) (177 mg, 0.5 mmol), DIPEA (143 mg, 0.19 ml, 1.1 mmol) and DCM (3.5 ml) was added and the resulting mixture was stirred at room temperature for 30 minutes. Cold water was then added and the product was extracted with EtOAc and the organic layer was washed with brine. The organic phase was dried over Na₂SO₄ and concentrated under reduced pressure to get the residue. The obtained residue was purified by column chromatography (using silica gel of 60-120 mesh and 50% EtOAc in Hexane as eluent) to afford 100 mg (39.2% yield 4-(2-Fluoro-phenyl)-pyrazole-1-carboxylic acid {2-oxo-2-[4-(4-trifluoromethyl-pyridine-3-carbonyl)-piperazin-1-yl]-ethyl}-amide. LCMS Purity: 97.68%. ¹H NMR (DMSO-d₆): δ 9.0-8.8 (dd, 2H), 8.7-8.6 (s, 1H), 8.5 (t, 1H), 8.3 (s, 1H), 7.9-7.8 (m, 2H), 7.4-7.2 (m, 3H), 4.3-4.1 (dd, 2H), 3.8-3.55 (m, 4H), 3.55-3.4 (m, 2H), 3.3-3.05 (m, 2H).

### Example 48

### 1-m-Tolyl-1H-[1,2,3]triazole-4-carboxylic acid {2-[4-(5-fluoro-2-trifluoromethyl-benzoyl)-piperazin-1-yl]-2-oxo-ethyl}-amide

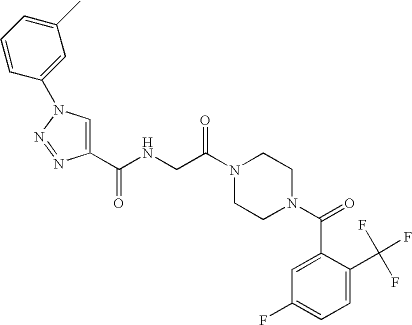

DIPEA (178 mg, 0.24 mL, 1.37 mmol) followed by HOBT (51 mg, 0.38 mmol) and EDCI (132 mg, 0.68 mmol) were added to a stirred solution of 1-m-Tolyl-1H-[1,2,3]triazole-4-carboxylic acid (70 mg, 0.29 mmol) (prepared according to a procedure similar to that described in steps 1-3 of General scheme-A from m-Tolylamine) in DMF (2 mL) at room temperature. After 2 minutes 2-Amino-1-[4-(5-fluoro-2-trifluoromethyl-benzoyl)-piperazin-1-yl]-ethanone hydrochloride salt (prepared according to a procedure similar to that described in synthesis procedure 1, using 5-Fluoro-2-trifluoromethyl-benzoic acid (Aldrich, St. Louis, Mo.) as a starting material) (120 mg, 0.32 mmol) was added and the resulting mixture was stirred at room temperature overnight. Cold water was then added and filtered the solid precipitated. The solid obtained was purified by recrystallisation from a mixture of DCM and hexane and further washing with diethyl ether to afford 83 mg (46.6%) of 1-m-Tolyl-1H-[1,2,3]triazole-4-carboxylic acid {2-[4-(5-fluoro-2-trifluoromethyl-benzoyl)-piperazin-1-yl]-2-oxo-ethyl}-amide. LCMS Purity: 94.55%. ¹H NMR (DMSO-d₆): δ 9.3 (s, 1H), 8.5 (t, 1H), 8.0-7.9 (m, 1H), 7.9-7.8 (s, 1H), 7.8-7.7 (d, 1H), 7.6-7.45 (m, 3H), 7.4-7.3 (d, 1H), 4.3-4.15 (dd, 2H), 3.8-3.4 (m, 6H), 3.3-3.1 (m, 2H), 2.4 (s, 3H).

### Example 49

### 1-m-Tolyl-1H-[1,2,3]triazole-4-carboxylic acid {2-[4-(2-chloro-5-fluoro-benzoyl)-piperazin-1-yl]-2-oxo-ethyl}-amide

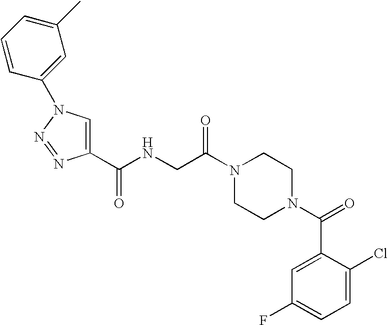

DIPEA (152.6 mg, 0.2 mL, 1.2 mmol) followed by HOBT (39.8 mg, 0.29 mmol) and EDCI (113 mg, 0.59 mmol) were added to a stirred solution of 1-m-Tolyl-1H-[1,2,3]triazole-4-carboxylic acid (60 mg, 0.29 mmol) (prepared according to a procedure similar to that described in steps 1-3 of General scheme-A from m-Tolylamine) in DMF (2 mL) at room temperature. After 2 minutes 2-Amino-1-[4-(2-chloro-5-fluoro-benzoyl)-piperazin-1-yl]-ethanone hydrochloride salt (prepared according to a procedure similar to that described in synthesis procedure 1, using 2-chloro-5-fluoro-benzoic acid (Aldrich, St. Louis, Mo.) as a starting material) (99 mg, 0.29 mmol) was added and the resulting mixture was stirred at room temperature overnight. Cold water was then added and filtered the solid precipitated. The solid obtained was purified by recrystallisation from a mixture of DCM and hexane and further washing with diethyl ether to afford 95 mg (66.4%) of 1-m-Tolyl-1H-[1,2,3]triazole-4-carboxylic acid {2-[4-(2-chloro-5-fluoro-benzoyl)-piperazin-1-yl]-2-oxo-ethyl}-amide. LCMS Purity: 95.26%. ¹H NMR (DMSO-d₆): δ 9.3 (s, 1H), 8.5 (t, 1H), 7.9-7.8 (s, 1H), 7.8-7.72 (d, 1H), 7.66-7.58 (m, 1H), 7.55-7.3 (m, 4H), 4.3-4.15 (dd, 2H), 3.8-3.46 (m, 6H), 3.26-3.16 (m, 2H), 2.4 (s, 3H).

### Example 50

1-o-Tolyl-1H-[1,2,3]triazole-4-carboxylic acid {2-[4-(5-fluoro-2-trifluoromethyl-benzoyl)-piperazin-1-yl]-2-oxo-ethyl}-amide

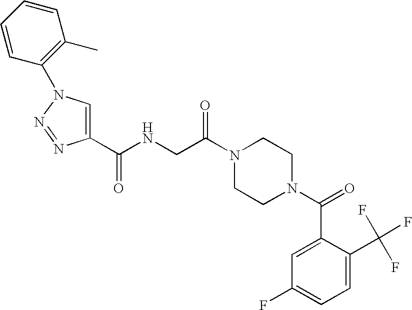

DIPEA (171 mg, 0.22 mL, 1.3 mmol) followed by HOBT (44 mg, 0.32 mmol) and EDCI (141 mg, 0.73 mmol) were added to a stirred solution of 1-o-Tolyl-1H-[1,2,3]triazole-4-carboxylic acid (60 mg, 0.29 mmol) (prepared according to a procedure similar to that described in steps 1-3 of General scheme-A from o-Tolylamine) in DMF (5 mL) at room temperature. After 2 minutes 2-Amino-1-[4-(5-fluoro-2-trifluoromethyl-benzoyl)-piperazin-1-yl]-ethanone hydrochloride salt (prepared according to a procedure similar to that described in synthesis procedure 1, using 5-Fluoro-2-trifluoromethyl-benzoic acid (Aldrich, St. Louis, Mo.) as a starting material) (120 mg, 0.32 mmol) was added and the resulting mixture was stirred at room temperature overnight. Cold water was then added and filtered the solid precipitated. The solid obtained was purified from a mixture of 20% EtOAc in hexane, 3 drops of water and 3 drops of MeOH to afford 133 mg (86.9%) of 1-o-Tolyl-1H-[1,2,3]triazole-4-carboxylic acid {2-[4-(5-fluoro-2-trifluoromethyl-benzoyl)-piperazin-1-yl]-2-oxo-ethyl}-amide. LCMS Purity: 97.81%. ¹H NMR (DMSO-d₆): δ 9.2 (s, 1H), 8.56 (t, 1H), 7.98 (t, 1H), 7.64-7.4 (m, 6H), 4.3 (d, 2H), 3.82-3.56 (m, 4H), 3.5 (s, 2H), 3.26 (d, 2H), 2.2 (s, 3H).

### Example 51

### 1-m-Tolyl-1H-[1,2,3]triazole-4-carboxylic acid {2-oxo-2-[4-(3,4,5-trifluoro-benzoyl)-piperazin-1-yl]-ethyl}-amide

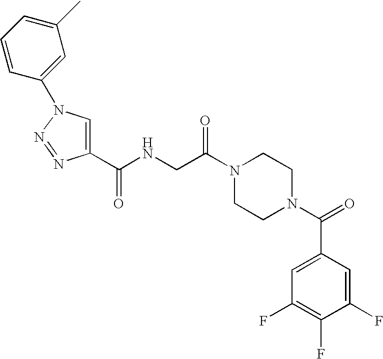

DIPEA (152 mg, 0.2 mL, 1.2 mmol) followed by HOBT (40 mg, 0.29 mmol) and EDCI (113 mg, 0.59 mmol) were added to a stirred solution of 1-m-Tolyl-1H-[1,2,3]triazole-4-carboxylic acid (60 mg, 0.29 mmol) (prepared according to a procedure similar to that described in steps 1-3 of General scheme-A from m-Tolylamine) in DMF (2 mL) at room temperature. After 2 minutes 2-Amino-1-[4-(3,4,5-trifluoro-benzoyl)-piperazin-1-yl]-ethanone hydrochloride salt (prepared according to a procedure similar to that described in synthesis procedure 1, using 3,4,5-trifluoro-benzoic acid (Aldrich, St. Louis, Mo.) as a starting material) (99.7 mg, 0.29 mmol) was added and the resulting mixture was stirred at room temperature overnight. Cold water was then added and filtered the solid precipitated. The solid obtained was purified by recrystallisation from a mixture of DCM and hexane and further washing with diethyl ether to afford 133 mg (86.9%) of 1-m-Tolyl-1H-[1,2,3]triazole-4-carboxylic acid {2-oxo-2-[4-(3,4,5-trifluoro-benzoyl)-piperazin-1-yl]-ethyl}-amide. LCMS Purity: 93.49%. ¹H NMR (DMSO-d₆): δ 9.35 (s, 1H), 8.6-8.5 (t, 1H), 7.9 (s, 1H), 7.8 (d, 1H), 7.6-7.5 (t, 3H), 7.4 (d, 1H), 4.3 (s, 2H), 3.8-3.6 (m, 6H), 3.5 (m, 2H), 2.5 (s, 3H).

### Example 52

### 1-(2-Cyano-phenyl)-1H-[1,2,3]triazole-4-carboxylic acid {2-[4-(2-chloro-5-fluoro-benzoyl)-piperazin-1-yl]-2-oxo-ethyl}-amide

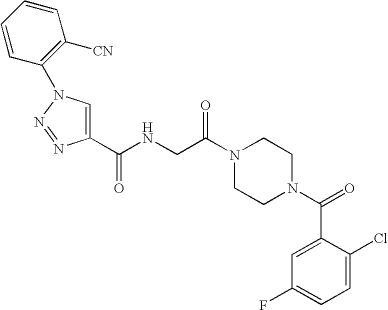

DIPEA (145 mg, 0.2 mL, 1.1 mmol) followed by HOBT (41.6 mg, 0.3 mmol) and EDCI (107 mg, 0.5 mmol) were added to a stirred solution of 1-(2-Cyano-phenyl)-1H-[1,2,3]triazole-4-carboxylic acid (60 mg, 0.28 mmol) (prepared according to a procedure similar to that described in steps 1-3 of General scheme-A from 2-Amino-benzonitrile) in DMF (2 mL) at room temperature. After 2 minutes 2-Amino-1-[4-(2-chloro-5-fluoro-benzoyl)-piperazin-1-yl]-ethanone hydrochloride salt (prepared according to a procedure similar to that described in synthesis procedure 1, using 2-chloro-5-fluoro-benzoic acid (Aldrich, St. Louis, Mo.) as a starting material) (94 mg, 0.28 mmol) was added and the resulting mixture was stirred at room temperature overnight. Cold water was then added and filtered the solid precipitated. The solid obtained was purified by recrystallisation from a mixture of DCM and hexane and further washing with diethyl ether to afford 45 mg (32.3%) of 1-(2-Cyano-phenyl)-1H-[1,2,3]triazole-4-carboxylic acid {2-[4-(2-chloro-5-fluoro-benzoyl)-piperazin-1-yl]-2-oxo-ethyl}-amide. LCMS Purity: 97.46%. ¹H NMR (DMSO-d₆): δ 9.2 (s, 1H), 8.6 (t, 1H), 8.2 (d, 1H), 8.0 (m, 2H), 7.8 (m, 1H), 7.6 (m, 1H), 7.5-7.3 (m, 3H), 4.4-4.2 (dd, 2H), 3.8 (m, 2H), 3.6 (m, 2H), 3.5 (m, 2H), 3.2 (m, 2H).

### Example 53

### 5-(4-Fluoro-phenyl)-isoxazole-3-carboxylic acid {2-[4-(2-chloro-5-fluoro-benzoyl)-piperazin-1-yl]-2-oxo-ethyl}-amide

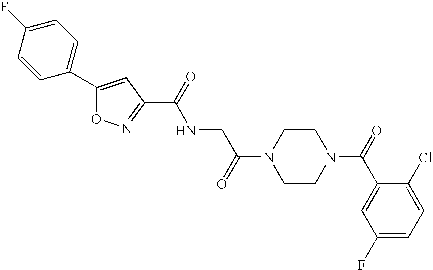

DIPEA (164 mg, 0.22 mL, 1.26 mmol) followed by HOBT (51.4 mg, 0.38 mmol) and EDCI (73 mg, 0.38 mmol) were added to a stirred solution of 5-(4-Fluoro-phenyl)-isoxazole-3-carboxylic acid (75 mg, 0.36 mmol) (prepared according to a procedure similar to that described in synthesis procedure 3, steps 1-4-b, using 4′-Fluoro-acetophenone (Aldrich, St. Louis, Mo.) as starting material) in DMF (2 mL) at room temperature. After 2 minutes 2 minutes 2-Amino-1-[4-(2-chloro-5-fluoro-benzoyl)-piperazin-1-yl]-ethanone hydrochloride salt (prepared according to a procedure similar to that described in synthesis procedure 1, using 2-chloro-5-fluoro-benzoic acid (Aldrich, St. Louis, Mo.) as a starting material) (122 mg, 0.36 mmol) was added and the resulting mixture was stirred at room temperature overnight. Cold water was then added and extracted with ethyl acetate. The organic layer was washed with brine and dried over Na₂SO₄, concentrated under reduced pressure to afford the residue. The residue obtained was purified by column chromatography (using 60-120 silica gel and 40% EtOAc in Hexane as eluent) to afford 42 mg (23.7%) of 5-(4-Fluoro-phenyl)-isoxazole-3-carboxylic acid {2-[4-(2-chloro-5-fluoro-benzoyl)-piperazin-1-yl]-2-oxo-ethyl}-amide. LCMS Purity: 88.2%. ¹H NMR (DMSO-d₆): δ 8.64 (t, 1H), 7.98 (m, 2H), 7.58 (m, 1H), 7.32 (m, 5H), 7.38 (m, 1H), 4.12 (dd, 2H), 3.46 (m, 6H), 3.14 (m, 2H).

### Example 54

6-Pyrazol-1-yl-imidazo[1,2-a]pyridine-2-carboxylic acid {2-[4-(2-chloro-5-fluoro-benzoyl)-piperazin-1-yl]-2-oxo-ethyl}-amide

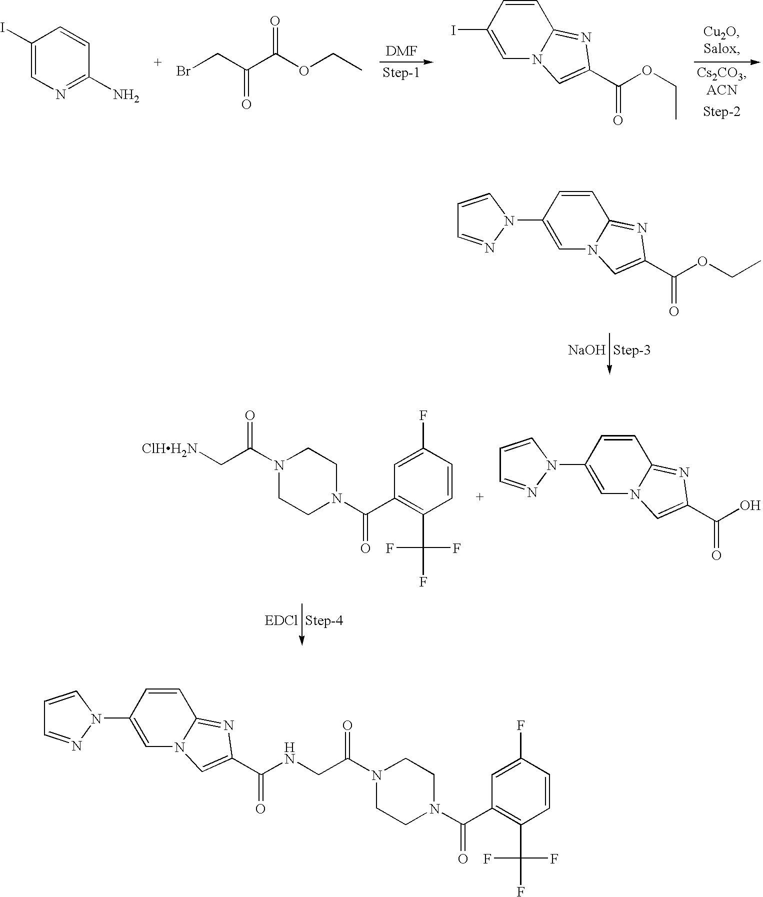

### Step 1

### Synthesis of 6-Iodo-imidazo[1,2-a]pyridine-2-carboxylic acid ethyl ester

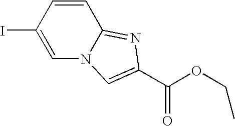

The title compound is prepared by similar method described in procedure, step-1 of example-28 using 5-Iodo-2-amino-pyridine (Aldrich, St. Louis. MO) as starting material.

### Step 2

### Synthesis of 6-Pyrazol-1-yl-imidazo[1,2-a]pyridine-2-carboxylic acid ethyl ester

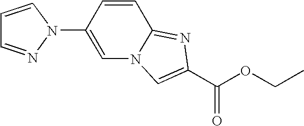

A mixture of Pyrazole (513 mg, 7.5 mmol), 6-Iodo-imidazo[1,2-a]pyridine-2-carboxylic acid ethyl ester (1.6 g, 5.03 mmol), Cu₂O (71 mg, 0.5 mmol), salox (137 mg, 1.0 mmol), Cs₂CO₃(3.27 g, 10.06 mmol) in acetonitrile (5 mL) in a microwave tube was purged with N₂ gas for 10 minutes. After which, subjected to microwave irradiation at 85° C. (Biotage microwave reactor), for 15 min(zero power and zero pressure). The reaction mixture was diluted with ethylacetate, washed with water, followed by brine solution. The organic layer was separated, dried over sodium sulphate, concentrated under reduced pressure to afford the residue. The residue thus obtained was purified by column chromatography (using neutral alumina and 0.2% MeOH in DCM as eluent) to afford 400 mg (31%) of 6-Pyrazol-1-yl-imidazo[1,2-a]pyridine-2-carboxylic acid ethyl ester.

### Step 3

### Synthesis of 6-Pyrazol-1-yl-imidazo[1,2-a]pyridine-2-carboxylic acid

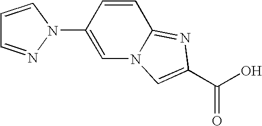

A mixture of 6-Pyrazol-1-yl-imidazo[1,2-a]pyridine-2-carboxylic acid ethyl ester (250 mg, 0.97 mmol) in 8N aqueous HCl solution (3 mL) was heated to 100° C. for 2 hr. The reaction mixture was concentrated to afford 130 mg (58.5%) of 6-Pyrazol-1-yl-imidazo[1,2-a]pyridine-2-carboxylic acid.

### Step 4

6-Pyrazol-1-yl-imidazo[7,2-a]pyridine-2-carboxylic acid {2-[4-(2-chloro-5-fluoro-benzoyl)-piperazin-1-yl]-2-oxo-ethyl}-amide

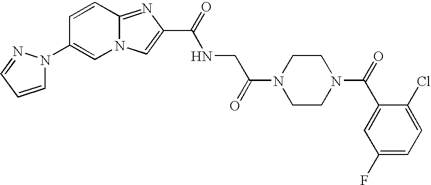

DIPEA (226 mg, 0.3 mL, 1.75 mmol) followed by HOBT (52 mg, 0.38 mmol) and EDCI (100 mg, 0.52 mmol) were added to a stirred solution of 6-Pyrazol-1-yl-imidazo[1,2-a]pyridine-2-carboxylic acid (80 mg, 0.35 mmol) in DMF (1 mL) at room temperature. After 2 minutes 2 minutes 2-Amino-1-[4-(2-chloro-5-fluoro-benzoyl)-piperazin-1-yl]-ethanone hydrochloride salt (prepared according to a procedure similar to that described in synthesis procedure 1, using 2-chloro-5-fluoro-benzoic acid (Aldrich, St. Louis, Mo.) as a starting material) (117 mg, 0.35 mmol) was added and the resulting mixture was stirred at room temperature overnight. Cold water was then added and extracted with ethyl acetate. The organic layer was washed with brine and dried over Na₂SO₄, concentrated under reduced pressure to afford the residue. The residue obtained was purified by preparative HPLC to afford 38 mg (20%) of 6-Pyrazol-1-yl-imidazo[1,2-a]pyridine-2-carboxylic acid {2-[4-(2-chloro-5-fluoro-benzoyl)-piperazin-1-yl]-2-oxo-ethyl}-amide. LCMS Purity: 96.78%. ¹H NMR (DMSO-d₆): δ 8.92 (s, 1H), 8.5 (dd, 2H), 8.3 (t, 1H), 8.0 (dd, 1H), 7.8 (d, 2H), 7.6 (m, 1H), 7.4 (m, 2H), 4.2 (dd, 2H), 3.7 (m, 4H), 3.5 (m, 2H), 3.2 (m, 2H).

### Example 55

### 4-(5-Fluoro-2-trifluoromethyl-benzoyl)-1-{2-[(5-phenyl-1H-pyrazole-3-carbonyl)-amino]-acetyl}-piperazine-2-carboxylic acid

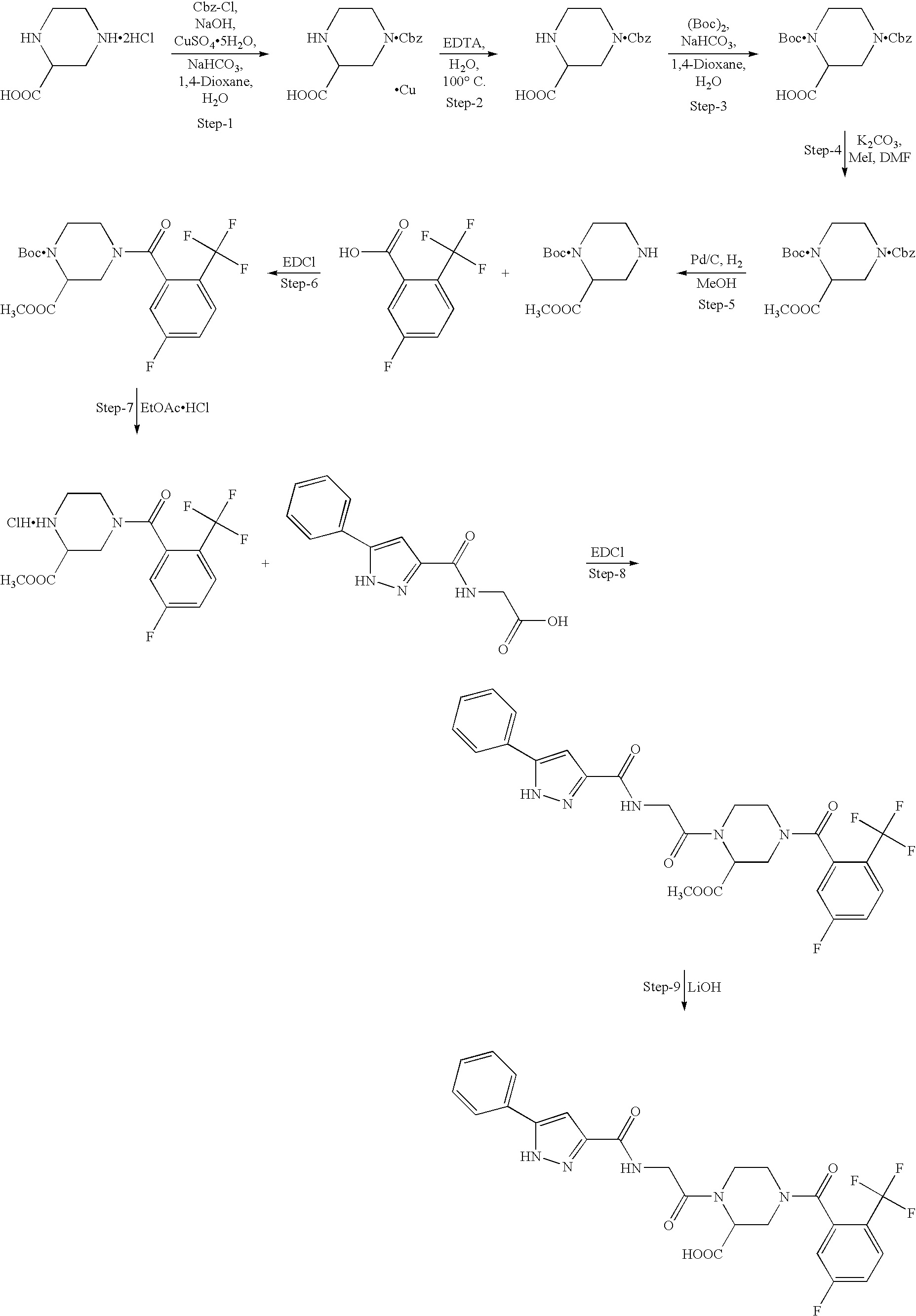

### Step 1

### Synthesis of piperazine-1,3-dicarboxylic acid 1-benzyl ester copper complex

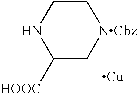

Aqueous 2.5M NaOH solution (4 mL) was added to a clear solution of piperazine-2-carboxylic acid dihydrochloride (1.0 g, 4.9 mmol) in water (8 mL) followed by addition of CuSO₄.5H₂O (635 mg, 2.46 mmol) in water (8 mL) dropwise. The resultant deep blue solution was cooled to 0° C. and was added solid NaHCO₃ (496 mg, 5.9 mmol) and stirred or 5 minutes. Cbz-Cl (921 mg, 5.4 mmol) in 1,4-dioxane (4 mL) was added to the above mixture dropwise and stirred for 16 hr. The pH was maintained >7 throughout the reaction. The reaction mass was filtered and the solid obtained was dried under vacuum to afford 1.6 g of piperazine-1,3-dicarboxylic acid 1-benzyl ester copper complex.

### Step 2

### Synthesis of piperazine-1,3-dicarboxylic acid 1-benzyl ester

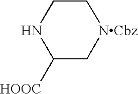

EDTA (800 mg) in water (25 mL) was added to piperazine-1,3-dicarboxylic acid 1-benzyl ester copper complex and heated to 100° C. for 5 hr. The reaction mass was concentrated under reduced pressure to afford 1.9 g of piperazine-1,3-dicarboxylic acid 1-benzyl ester.

### Step 3

### Synthesis of piperazine-1,2,4-tricarboxylic acid 4-benzyl ester 1-tert-butyl ester

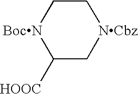

NaHCO₃(1.9 g, 22.6 mmol) was added to a stirred solution of piperazine-1,3-dicarboxylic acid 1-benzyl ester (1.9 g, 7.2 mmol) in water (10 mL) and stirring was continued for 30 minutes at ambient temperature. To the above mixture at 0° C., was added di-tert-butyl dicarbonate (4 g, 19.8 mmol) dropwise and stirring was continued at ambient temperature overnight. The reaction mixture was diluted with cold water and further acidified with aqueous 2N HCl to pH 5 and extracted with ethyl acetate and dried over sodium sulphate, concentrated under reduced pressure to afford the residue. The residue obtained was purified by column chromatography (using 60-120 silica gel and 1% MeOH in CHCl₃ as eluent) to afford 1.25 g (48%) of piperazine-1,2,4-tricarboxylic acid 4-benzyl ester 1-tert-butyl ester.

### Step 4

### Synthesis of piperazine-1,2,4-tricarboxylic acid 4-benzyl ester 1-tert-butyl ester 2-methyl ester

K₂CO₃ (910 mg, 6.6 mmol) was added to a stirred solution of piperazine-1,2,4-tricarboxylic acid 4-benzyl ester 1-tert-butyl ester (1.2 g, 3.3 mmol) in DMF (10 mL) at ambient temperature. To the above mixture was added at 0° C., CH₃I (1.4 g, 9.9 mmol) and stirred 20° C. for 2 hr. The reaction mixture was diluted with cold water, extracted with ethyl acetate and dried over sodium sulphate, concentrated under reduced pressure to afford 1.2 g(96.4%) of piperazine-1,2,4-tricarboxylic acid 4-benzyl ester 1-tert-butyl ester 2-methyl ester.

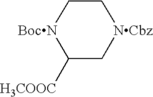

### Step 5

### Synthesis of piperazine-1,2-dicarboxylic acid 1-tert-butyl ester 2-methyl ester

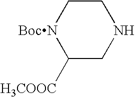

10% Pd/C (250 mg) was added to a solution of piperazine-1,2,4-tricarboxylic acid 4-benzyl ester 1-tert-butyl ester 2-methyl ester (1.2 g, 3.173 mmol) in MeOH (15 mL) under inert atmosphere and stirring was continued under hydrogen atmosphere for 2 hrs. The reaction mixture was filtered over celite, filtrate collected was concentrated under reduced pressure to afford 650 mg (83.97%) of piperazine-1,2-dicarboxylic acid 1-tert-butyl ester 2-methyl ester.

### Step 6

### Synthesis of 4-(5-Fluoro-2-trifluoromethyl-benzoyl)-piperazine-1,2-dicarboxylic acid 1-tert-butyl ester 2-methyl ester

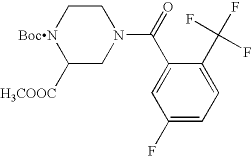

DIPEA (430 mg, 0.58 mL, 3.27 mmol) followed by HOBT (121 mg, 0.9 mmol) and EDCI (230 mg, 1.22 mmol) were added to a stirred solution of 5-Fluoro-2-trifluoromethyl-benzoic acid (170 mg, 0.82 mmol) in DMF (2.0 mL) at room temperature. After 2 minutes 2 minutes piperazine-1,2-dicarboxylic acid 1-tert-butyl ester 2-methyl ester (200 mg, 0.82 mmol) was added and the resulting mixture was stirred at room temperature overnight. Cold water was then added, filtered the solid precipitated to afford 305 mg (85.9%) of 4-(5-Fluoro-2-trifluoromethyl-benzoyl)-piperazine-1,2-dicarboxylic acid 1-tert-butyl ester 2-methyl ester.

### Step 7

### Synthesis of 4-(5-Fluoro-2-trifluoromethyl-benzoyl)-piperazine-2-carboxylic acid methyl ester hydrochloride

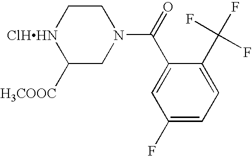

A mixture of 4-(5-Fluoro-2-trifluoromethyl-benzoyl)-piperazine-1,2-dicarboxylic acid 1-tert-butyl ester 2-methyl ester (300 mg, 0.69 mmol) in EtOAc-HCl (5 mL) was stirred at 0° C. for 2 hr. The reaction mixture was concentrated under reduced pressure to afford the residue. The residue obtained was washed with diethyl ether to afford 250 mg (97.6%) of 4-(5-Fluoro-2-trifluoromethyl-benzoyl)-piperazine-2-carboxylic acid methyl ester hydrochloride.

### Step 8

### Synthesis of 4-(5-Fluoro-2-trifluoromethyl-benzoyl)-1-{2-[(5-phenyl-1H-pyrazole-3-carbonyl)-amino]-acetyl}-piperazine-2-carboxylic acid methyl ester

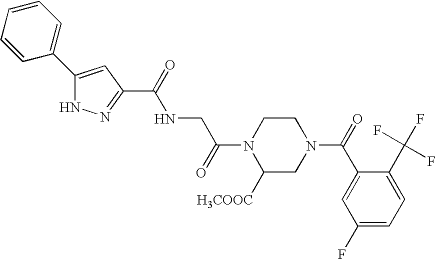

DIPEA (418 mg, 0.56 mL, 3.23 mmol) followed by HOBT (96 mg, 0.71 mmol) and EDCI (186 mg, 0.97 mmol) were added to a stirred solution of [(5-Phenyl-1H-pyrazole-3-carbonyl)-amino]-acetic acid (prepared according to a procedure similar to that described in synthesis procedure 4, Steps 1-3 and 5-6) (174 mg, 0.72 mmol) in DMF (2.5 mL) at room temperature. After 2 minutes 2 minutes 4-(5-Fluoro-2-trifluoromethyl-benzoyl)-piperazine-2-carboxylic acid methyl ester hydrochloride salt (240 mg, 0.64 mmol) was added and the resulting mixture was stirred at room temperature overnight. Cold water was then added, filtered the solid precipitated to afford 205 mg (56.4%) of 4-(5-Fluoro-2-trifluoromethyl-benzoyl)-1-{2-[(5-phenyl-1H-pyrazole-3-carbonyl)-amino]-acetyl}-piperazine-2-carboxylic acid methyl ester.

### Step 9

### Synthesis of 4-(5-Fluoro-2-trifluoromethyl-benzoyl)-1-{2-[(5-phenyl-1H-pyrazole-3-carbonyl)-amino]-acetyl}-piperazine-2-carboxylic acid

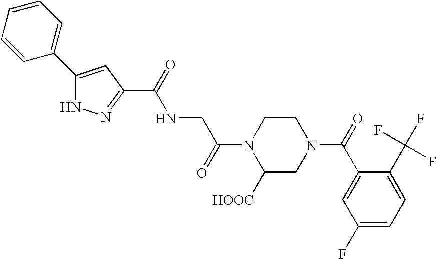

LiOH.H₂O (44 mg, 1.07 mmol) was added to a stirred solution of 4-(5-Fluoro-2-trifluoromethyl-benzoyl)-1-{2-[(5-phenyl-1H-pyrazole-3-carbonyl)-amino]-acetyl}-piperazine-2-carboxylic acid methyl ester (200 mg, 0.35 mmol) in THF:H2O:MeOH (2:1:0.6, 3.6 mL), and the resulting mixture was stirred at room temperature for 2 hr. The reaction mixture was concentrated under reduced pressure to get the residue. Cold water was then added and acidified it with 10% aqueous HCl, extracted with ethyl acetate. The organic layer collected was dried over sodium sulphate and concentrated under reduced pressure to afford the residue. The residue obtained was purified by preparative HPLC to afford 23 mg (11.8%) of 4-(5-Fluoro-2-trifluoromethyl-benzoyl)-1-{2-[(5-phenyl-1H-pyrazole-3-carbonyl)-amino]-acetyl}-piperazine-2-carboxylic acid. LCMS Purity: 91.14%. ¹H NMR (DMSO-d₆): δ 13.5 (bs, 1H), 8.2 (bs, 1H), 7.9 (bs, 1H), 7.8 (d, 2H), 7.6-7.3 (m, 5H), 7.2-7.0 (m, 1H), 5.1-4.8 (m, 2H), 4.5-4.3 (m, 2H), 4.2-3.5 (m, 4H), 3.1 (m, 1H).

### Example 56

### 1-(5-Fluoro-2-trifluoromethyl-benzoyl)-4-{2-[(5-phenyl-1H-pyrazole-3-carbonyl)-amino]-acetyl}-piperazine-2-carboxylic acid

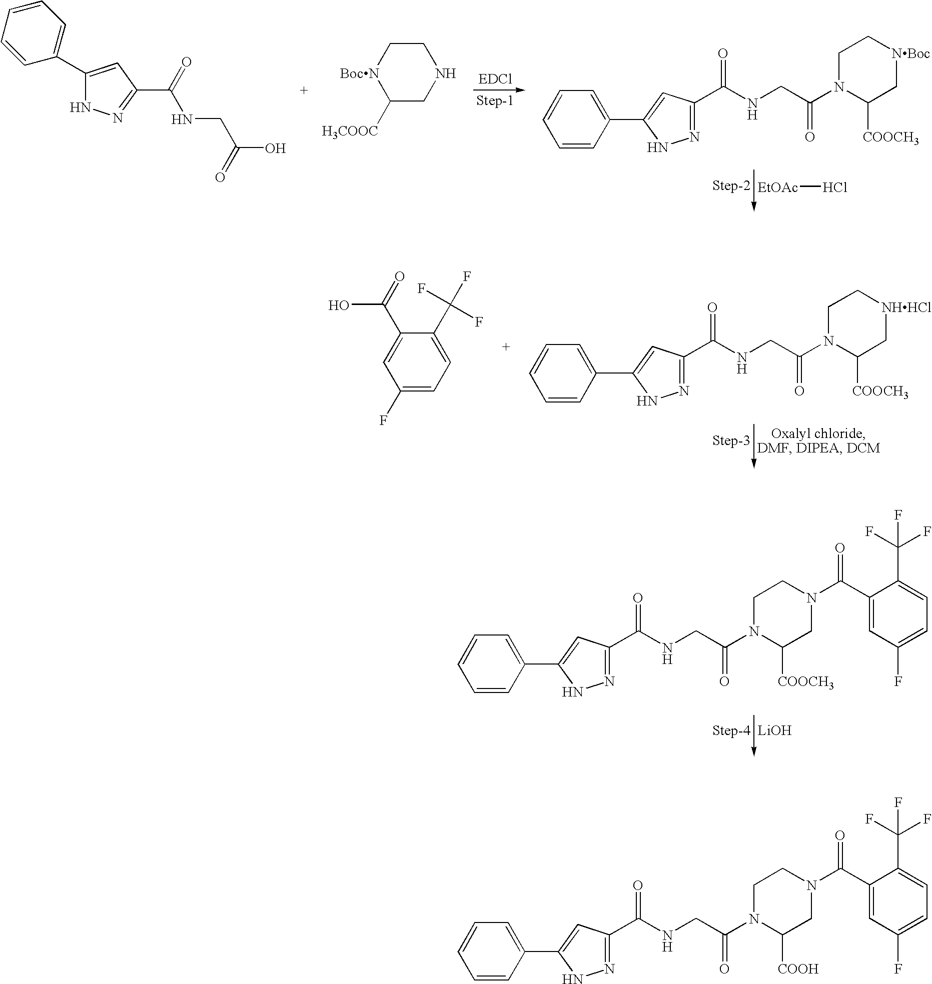

### Step 1

### Synthesis of 4-{2-[(5-Phenyl-1H-pyrazole-3-carbonyl)-amino]-acetyl}-piperazine-1,2-dicarboxylic acid 1-tert-butyl ester 2-methyl ester

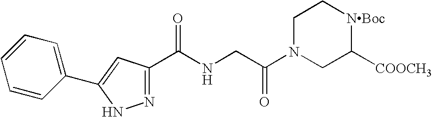

DIPEA (317 mg, 0.43 mL, 2.45 mmol) followed by HOBT (121 mg, 0.9 mmol) and EDCI (234 mg, 1.23 mmol) were added to a stirred solution of [(5-Phenyl-1H-pyrazole-3-carbonyl)-amino]-acetic acid (prepared according to a procedure similar to that described in example 25, Steps 1-3 and 5-6) (200 mg, 0.8 mmol) in DMF (2.0 mL) at room temperature. After 2 minutes piperazine-1,2-dicarboxylic acid 1-tert-butyl ester 2-methyl ester (200 mg, 0.8 mmol) was added and the resulting mixture was stirred at room temperature overnight. Cold water was then added, filtered the solid precipitated to afford 280 mg (72.53%) of 4-{2-[(5-Phenyl-1H-pyrazole-3-carbonyl)-amino]-acetyl}-piperazine-1,2-dicarboxylic acid 1-tert-butyl ester 2-methyl ester.

### Step 2

### Synthesis of 4-{2-[(5-Phenyl-1H-pyrazole-3-carbonyl)-amino]-acetyl}-piperazine-2-carboxylic acid methyl ester

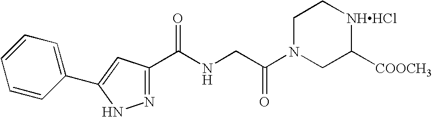

A mixture of 4-{2-[(5-Phenyl-1H-pyrazole-3-carbonyl)-amino]-acetyl}-piperazine-1,2-dicarboxylic acid 1-tert-butyl ester 2-methyl ester (290 mg, 0.61 mmol) in EtOAc-HCl (2 mL) was stirred at 0° C. for 2 hr. The reaction mixture was concentrated under reduced pressure to afford the residue. The residue obtained was washed with diethyl ether to afford 185 mg (73.7%) of 4-{2-[(5-Phenyl-1H-pyrazole-3-carbonyl)-amino]-acetyl}-piperazine-2-carboxylic acid methyl ester hydrochloride.

### Step 3

### Synthesis of 1-(5-Fluoro-2-trifluoromethyl-benzoyl)-4-{2-[(5-phenyl-1H-pyrazole-3-carbonyl)-amino]-acetyl}-piperazine-2-carboxylic acid methyl ester

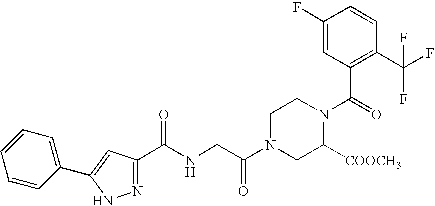

Oxalyl chloride (67 mg, 0.53 mmol) was added to an ice cold (0° C.) solution of 5-Fluoro-2-trifluoromethyl-benzoic acid (91 mg, 0.44 mmol) in DCM (3 mL) followed by 1 drop of DMF and stirred at ambient temperature for 30 minutes. The reaction mixture was concentrated under reduced pressure to afford the residue. The residue obtained was diluted with DCM (3 mL) and added to a cold (0° C.) mixture of 4-{2-[(5-Phenyl-1H-pyrazole-3-carbonyl)-amino]-acetyl}-piperazine-2-carboxylic acid methyl ester (180 mg, 0.44 mmol), DIPEA (171 mg, 0.23 mL, 1.32 mmol) in DCM (4 mL) and stirred at ambient temperature for 1 hr. The reaction mass was diluted with DCM, washed the organic layer with water followed by brine solution, dried over sodium sulphate and concentrated under reduced pressure to afford 200 mg(80.74%) of 1-(5-Fluoro-2-trifluoromethyl-benzoyl)-4-{2-[(5-phenyl-1H-pyrazole-3-carbonyl)-amino]-acetyl}-piperazine-2-carboxylic acid methyl ester.

### Step 4

### Synthesis of 1-(5-Fluoro-2-trifluoromethyl-benzoyl)-4-{2-[(5-phenyl-1H-pyrazole-3-carbonyl)-amino]-acetyl}-piperazine-2-carboxylic acid

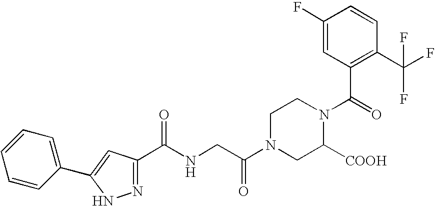

LiOH.H₂O (44 mg, 1.07 mmol) was added to a stirred solution of 1-(5-Fluoro-2-trifluoromethyl-benzoyl)-4-{2-[(5-phenyl-1H-pyrazole-3-carbonyl)-amino]-acetyl}-piperazine-2-carboxylic acid methyl ester (200 mg, 0.35 mmol) in THF:H2O:MeOH (2:1:0.6, 3.6 mL), and the resulting mixture was stirred at room temperature for 2 hr. The reaction mixture was concentrated under reduced pressure to get the residue. Cold water was then added and acidified it with 10% aqueous HCl to pH 7, extracted with ethyl acetate. The organic layer collected was dried over sodium sulphate and concentrated under reduced pressure to afford the residue. The residue obtained was purified by preparative HPLC to afford 30 mg (15.3%) 1-(5-Fluoro-2-trifluoromethyl-benzoyl)-4-{2-[(5-phenyl-1H-pyrazole-3-carbonyl)-amino]-acetyl}-piperazine-2-carboxylic acid. LCMS Purity: 98.5%. ¹H NMR (DMSO-d₆): δ 13.8 (bs, 1H), 8.2 (bs, 1H), 8.0 (m, 1H), 7.8 (d, 2H), 7.7 (d, 1H), 7.6 (d, 1H), 7.5 (t, 2H), 7.4 (t, 1H), 7.2 (s, 1H), 5.4-5.0 (m, 1H), 4.8 (m, 1H), 4.5 (m, 1H), 4.3-4.0 (m, 3H), 3.4-3.0 (m, 4H), 2.9-2.7 (m, 1H).

### Example 57

### 2-{2-Oxo-2-[4-(2-trifluoromethyl-benzoyl)-piperazin-1-yl]-acetylamino}-benzoic acid methyl ester

### Step 1

### Synthesis of 3-Amino-benzoic acid methyl ester

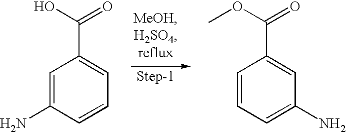

Concentrated H₂SO₄ (3 mL) was added to solution of 3-Amino-benzoic acid (7.5 g, 54.74 mmol) in MeOH (200 mL) and refluxed overnight. The reaction mixture was concentrated under reduced pressure to afford the residue. The residue was diluted with water, basified, extracted with ethyl acetate, washed with brine solution, dried over sodium sulfate and concentrated under reduced pressure to afford 2.7 g (33%) of 3-Amino-benzoic acid methyl ester. LCMS Purity: 98.9%.

### Step 2

### Synthesis of Oxo-[4-(2-trifluoromethyl-benzoyl)-piperazin-1-yl]-acetyl chloride

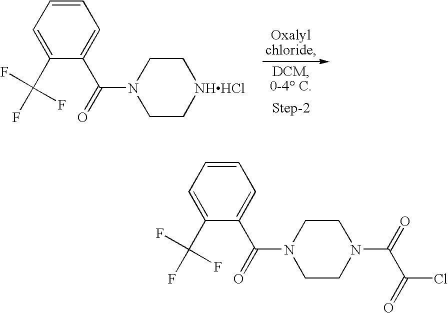

Oxalyl chloride (220 mg, 0.15 mL, 1.7 mmol) was added to cold (0-4° C.) solution of piperazin-1-yl-(2-trifluoromethyl-phenyl)-methanone hydrochloride salt (508 mg, 1.7 mmol) (prepared by the similar method as described in the earlier example using 2-Trifluoromethyl-benzoic acid as starting material Aldrich, St. Louis, Mo.) in DCM (18 mL) and stirring was continued at the same temperature for 1 hr to afford Oxo-[4-(2-trifluoromethyl-benzoyl)-piperazin-1-yl]-acetyl chloride.

### Step 3

### Synthesis of 2-{2-Oxo-2-[4-(2-trifluoromethyl-benzoyl)-piperazin-1-yl]-acetylamino}-benzoic acid methyl ester

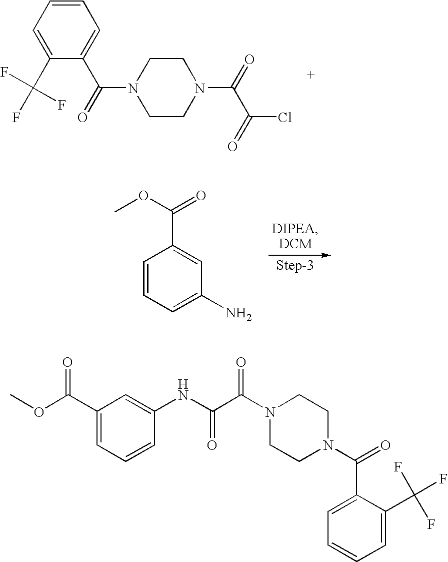

3-Amino-benzoic acid methyl ester (207 mg, 1.37 mmol) to the reaction mixture obtained from Step-2, followed by DIPEA (713 mg, 0.96 mL, 5.5 mmol) and stirring was continued at ambient temperature overnight. The reaction mixture was quenched with cold water, extracted with ethyl acetate and the organic layer collected was washed with aqueous saturated NaHCO₃ solution followed by brine solution. The organic layer thus collected was dried over sodium sulphate, concentrated under reduced pressure to afford the residue. The residue obtained was purified by column chromatography (using 60-120 silica gel and 60% EtOAc in Hexane as eluent) to afford 225 mg (28.66%) of 2-{2-Oxo-2-[4-(2-trifluoromethyl-benzoyl)-piperazin-1-yl]-acetylamino}-benzoic acid methyl ester. LCMS Purity: 96.23%. ¹H NMR (CDCl₃): δ 9.5 (d, 1H), 8.2 (d, 1H), 7.9-7.8 (m, 2H), 7.7 (d, 1H), 7.7-7.5 (m, 2H), 7.4 (q, 1H), 7.3 (d, 1H), 4.5 (d, 1H), 4.4-4.2 (m, 1H), 4.2-4.0 (m, 2H), 3.9 (d, 3H), 3.9-3.6 (m, 3H), 3.5 (b, 1H), 3.2 (s, 2H).

### Example 58

### 2-Oxo-N-(6-phenyl-pyridin-3-yl)-2-[4-(2-trifluoromethyl-benzoyl)-piperazin-1-yl]-acetamide

### Step 1

### Synthesis of 5-Nitro-2-phenyl-pyridine

Sodium carbonate (802 mg, 4.54 mmol) was added to a mixture of toluene (15 mL) and water (5 mL) and degassed with argon gas for 5 min. This was followed by the addition of phenyl boronic acid (587 mg, 4.54 mmol) and 2-Chloro-5-nitro-pyridine (600 mg, 3.78 mmol) and degassed the mixture with argon gas for 5 min. To the above resulting mixture added tetrakis palladium triphenyl phosphine (878 mg, 0.76 mmol) and degassed the mixture with argon gas for 5 min. The resulting reaction mixture was heated to reflux for 3 hrs. The reaction mixture was diluted with ethylacetate, washed with water followed by brine solution. The ethylacetate layer was dried over sodium sulphate and concentrated under reduced pressure to afford the residue. The residue obtained was purified by column chromatography using 60-120 silica gel and 5% Ethylacetate in Hexane as eluent) to afford 500 mg (66%) of 5-Nitro-2-phenyl-pyridine. LCMS purity: 98.2%

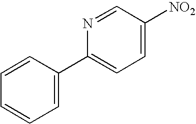

### Step 2

### Synthesis of 6-Phenyl-pyridin-3-ylamine

Ammonium chloride (1.1 g, 20.0 mmol) dissolved in water (15 mL) was added to a stirred solution of 5-Nitro-2-phenyl-pyridine (500 mg, 2.5 mmol) in THF (10 mL), followed by MeOH (5 mL) to afford a clear solution. This was followed by portion wise addition of Zinc powder (1.3 g, 20.0 mmol) at room temperature with stirring for 1 hr. Filtered the mixture over celite bed, concentrated the filtrate to afford the residue. The residue obtained was extracted with ethyl acetate, washed the organic layer with brine solution, dried over Na₂SO₄, and concentrated under reduced pressure to afford 350 mg (82%) of 6-Phenyl-pyridin-3-ylamine. LCMS purity: 87.9%

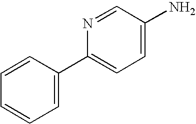

### Step 3

### Synthesis of Oxo-[4-(2-trifluoromethyl-benzoyl)-piperazin-1-yl]-acetyl chloride

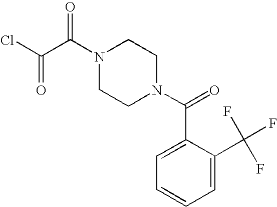

The preparation of title compound follows the similar method as mentioned in example 57, Step-2 to afford Oxo-[4-(2-trifluoromethyl-benzoyl)-piperazin-1-yl]-acetyl chloride

### Step 4

### Synthesis of 2-Oxo-N-(6-phenyl-pyridin-3-yl)-2-[4-(2-trifluoromethyl-benzoyl)-piperazin-1-yl]-acetamide

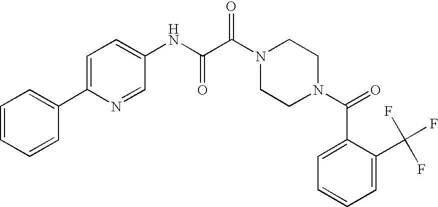

The preparation of title compound follows the similar method as mentioned in example 57, Step-3.

The residue obtained was initially purified by column chromatography (60-120 silica gel and 1% MeOH in CHCl₃ as eluent) and further purified by preparative HPLC to afford 28 mg (17.17%) of 2-Oxo-N-(6-phenyl-pyridin-3-yl)-2-[4-(2-trifluoromethyl-benzoyl)-piperazin-1-yl]-acetamide. LCMS Purity: 95.71%. ¹H NMR (DMSO-d₆): δ 11.2 (d, 1H), 9.0-8.8 (d, 1H), 8.3-8.1 (m, 1H), 8.1-7.9 (m, 3H), 7.9-7.6 (m, 3H), 7.6-7.4 (m, 4H), 3.9-3.6 (m, 6H), 3.3-3.1 (m, 3H).

### Example 59

### N-Biphenyl-4-yl-2-[4-(5-fluoro-2-trifluoromethyl-benzoyl)-piperazin-1-yl]-2-oxo-acetamide

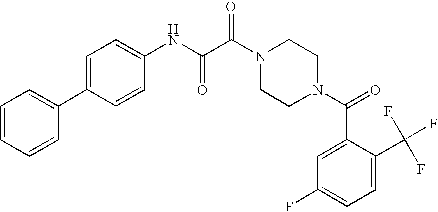

The preparation follows similar method as described in example 58 Step-3 and Step-4 (using Biphenyl-4-ylamine as starting material, Aldrich, St. Louis, Mo.) to afford 38 mg(15.8%) of N-Biphenyl-4-yl-2-[4-(5-fluoro-2-trifluoromethyl-benzoyl)-piperazin-1-yl]-2-oxo-acetamide. LCMS Purity: 97.87%. ¹H NMR (DMSO-d₆): δ 10.9 (d, 1H), 8.0-7.9 (m, 1H), 7.8-7.6 (m, 6H), 7.6-7.4 (m, 4H), 7.4-7.3 (m, 1H), 3.9-3.6 (m, 2H), 3.6 (m, 2H), 3.6-3.4 (m, 2H), 3.3-3.1 (m, 2H).

### Example 60

### Step 1

### [4-(5-Fluoro-2-trifluoromethyl-benzoyl)-piperazin-1-yl]-oxo-acetic acid ethyl ester

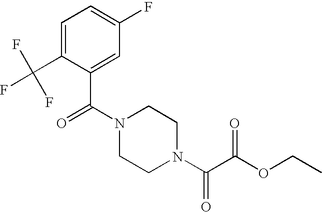

Chloro-oxo-acetic acid ethyl ester (480 mg, 3.51 mmol) was added a cold (0° C.) mixture of 5-Fluoro-2-trifluoromethyl-phenyl)-piperazin-1-yl-methanone hydrochloride salt (1.0 g, 3.2 mmol) prepared by the similar method as described in earlier example using 5-fluoro-2-trifluoromethyl benzoic acid as starting material, Aldrich, St. Louis, Mo.), Et₃N (809 mg, 1.1 mL, 8.0 mmol) in DCM (10 mL) and stirring was continued at ambient temperature for 1 hr. The reaction mixture was diluted with water, extracted with ethyl acetate, washed the organic layer with brine solution, dried over Na₂SO₄, and concentrated under reduced pressure to afford 1.1 g (91.4%) of [4-(5-Fluoro-2-trifluoromethyl-benzoyl)-piperazin-1-yl]-oxo-acetic acid ethyl ester.

### Step 2

### Synthesis of [4-(5-Fluoro-2-trifluoromethyl-benzoyl)-piperazin-1-yl]-oxo-acetic acid

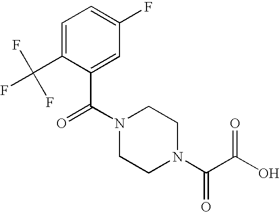

NaOH (53 mg, 1.32 mmol) in water (10 mL) was added to a stirred cold(0° C.) solution of [4-(5-Fluoro-2-trifluoromethyl-benzoyl)-piperazin-1-yl]oxo-acetic acid ethyl ester (100 mg, 0.26 mmol) in THF (10 mL) and the resulting mixture was stirred at ambient temperature for 1 hr. The reaction mixture was bubbled with N₂ gas to remove THF to afford the residue. The pH of the residue was adjusted to ‘5’ using 1N HCl. The aqueous layer was extracted with diethyl ether, washed with water, followed by brine solution. The organic layer thus collected dried over sodium sulphate, concentrated under reduced pressure to afford 40 mg (43.2%) of [4-(5-Fluoro-2-trifluoromethyl-benzoyl)-piperazin-1-yl]-oxo-acetic acid.

### Step 3

### Synthesis of [4-(5-Fluoro-2-trifluoromethyl-benzoyl)-piperazin-1-yl]-oxo-acetyl chloride

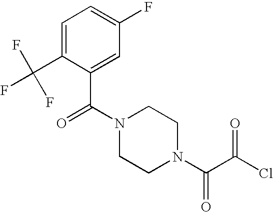

Oxalyl chloride (43 mg, 0.34 mmol) was added to an ice cold (0° C.) solution of [4-(5-Fluoro-2-trifluoromethyl-benzoyl)-piperazin-1-yl]-oxo-acetic acid (100 mg, 0.28 mmol) in DCM (3 mL) followed by 1 drop of DMF and stirred at ambient temperature for 30 minutes. The reaction mixture was concentrated under reduced pressure at ambient temperature under dry condition to afford 105 mg of [4-(5-Fluoro-2-trifluoromethyl-benzoyl)-piperazin-1-yl]-oxo-acetyl chloride.

### Step 4

### Synthesis of N-Biphenyl-2-yl-2-[4-(5-fluoro-2-trifluoromethyl-benzoyl)-piperazin-1-yl]-2-oxo-acetamide

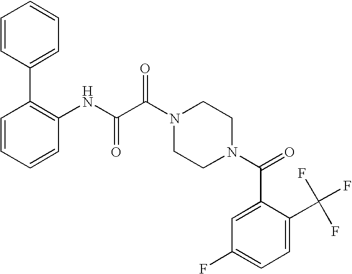

The preparation follows similar method as described in example 60, Step-1 to 3 and Step 4 (using 2-amino biphenyl as starting material, Aldrich, St. Louis., Mo.) to afford 55 mg(38.46%) of N-Biphenyl-2-yl-2-[4-(5-fluoro-2-trifluoromethyl-benzoyl)-piperazin-1-yl]-2-oxo-acetamide. LCMS Purity: 95.99%. ¹H NMR (DMSO-d₆): δ 10.2 (d, 1H), 8.0 (m, 1H), 7.6-7.45 (m, 3H), 7.45-7.3 (m, 7H), 7.2 (m, 1H), 3.8-3.4 (m, 4H), 3.2-2.8 (m, 4H).

### Example 61

### 3-{2-[4-(5-Fluoro-2-trifluoromethyl-benzoyl)-piperazin-1-yl]-2-oxo-acetylamino}-N-methyl-benzamide

### Step 1

### Synthesis of 3-Nitro-benzoyl chloride

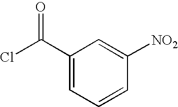

Oxalyl chloride (98.7 g, 777 mmol) was added to an ice cold (0° C.) solution of 3-nitro benzoic acid (100 g, 598 mmol) in DCM (400 mL) followed by DMF (0.5 mL) and stirred at ambient temperature for 4 hr. The reaction mixture was concentrated under reduced pressure to afford 111 g of 3-Nitro-benzoyl chloride.

### Step 2

### Synthesis of N-Methyl-3-nitro-benzamide

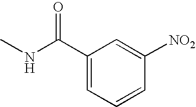

3-Nitro-benzoyl chloride (111 g, 598 mmol) was added dropwise to a cold (0° C.) solution of Methyl amine solution (40% aqueous solution) (55.72 g, 1794 mmol) in water (300 mL), filtered the solid precipitated to afford 86.34 g (80.16%) of N-Methyl-3-nitro-benzamide. LCMS Purity: 93%.

### Step 3

### Synthesis of 3-Amino-N-methyl-benzamide

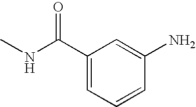

Ammonium chloride (254 g, 4793 mmol) dissolved in water (1000 mL) was added to a stirred solution of N-Methyl-3-nitro-benzamide (86.3 g, 479 mmol) in THF (700 mL), followed by MeOH (300 mL) to afford a clear solution. This was followed by portion wise addition of Zinc powder (245 g, 3834 mmol) with stirring and temperature while addition of zinc was maintained below 50° C. The reaction mixture was stirred at ambient temperature for 3 hr. filtered the mixture over celite bed, concentrated the filtrate to afford the residue. The residue obtained was extracted with ethyl acetate, washed with brine solution, dried over Na₂SO₄, and concentrated under reduced pressure to afford the crude solid. The crude solid was purified by recrystallisation from ethyl acetate to afford 63 g (87.6%) of 3-Amino-N-methyl-benzamide. LCMS purity: 96.26%.

### Step 4

### Synthesis of 3-{2-[4-(5-Fluoro-2-trifluoromethyl-benzoyl)-piperazin-1-yl]-2-oxo-acetylamino}-N-methyl-benzamide

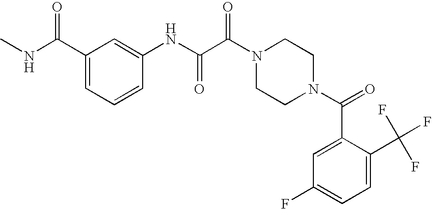

[4-(5-Fluoro-2-trifluoromethyl-benzoyl)-piperazin-1-yl]-oxo-acetyl chloride (105 mg, 0.286 mmol) (prepared by the similar method described in example 57, Step-2 using 5-Fluoro-2-trifluoromethyl-phenyl)-piperazin-1-yl-methanone hydrochloride salt as starting material which is being prepared by the general method mentioned in the earlier examples using 5-fluoro-2-trifluoromethyl benzoic acid as starting material, Aldrich, St. Louis, Mo.) in DCM (2.5 mL) was added to cold(0° C.) solution of 3-Amino-N-methyl-benzamide (43 mg, 0.28 mmol), Et₃N (86 mg, 0.12 mL, 0.86 mmol) in DCM (2.5 mL) and stirring was continued at ambient temperature for 2 hr. The reaction mixture was diluted with DCM, washed with cold water, followed by 2N HCl and brine solution. The organic layer thus collected was dried over sodium sulphate, concentrated under reduced pressure to afford the residue. The residue obtained was purified by recrystallisation from a mixture of ethyl acetate and hexane to afford 70 mg (51.09%) of 3-{2-[4-(5-Fluoro-2-trifluoromethyl-benzoyl)-piperazin-1-yl]-2-oxo-acetylamino}-N-methyl-benzamide. LCMS Purity: 99.7%. ¹H NMR (DMSO-d₆): δ 10.95 (d, 1H), 8.4 (m, 1H), 8.2-8.0 (d, 1H), 8.6-7.9 (m, 1H), 7.8-7.6 (m, 1H), 7.6-7.4 (m, 4H), 3.9-3.4 (m, 6H), 3.3-3.1 (m, 2H), 2.8 (m, 3H).

### Example 62

2-[4-(5-Fluoro-2-trifluoromethyl-benzoyl)-piperazin-1-yl]-2-oxo-N-(4-phenoxy-phenyl)-acetamide

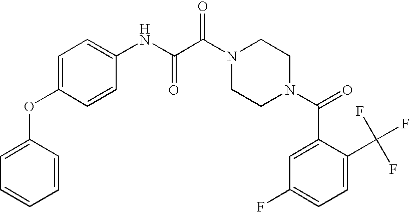

The preparation follows similar method as described in example 58, Step-4 using 4-phenoxy phenyl amine as starting material to afford 60 mg (40.67%) of 2-[4-(5-Fluoro-2-trifluoromethyl-benzoyl)-piperazin-1-yl]-2-oxo-N-(4-phenoxy-phenyl)-acetamide. LCMS Purity: 98.65%. ¹H NMR (DMSO-d₆): δ 10.8 (d, 1H), 7.9 (m, 1H), 7.5-7.3 (m, 4H), 7.4 (m, 2H), 7.2-7.0 (m, 5H), 3.9-3.4 (m, 6H), 3.2 (m, 2H).

### Example 63

### Step 1

### Synthesis of N-Biphenyl-4-yl-oxalamic acid ethyl ester

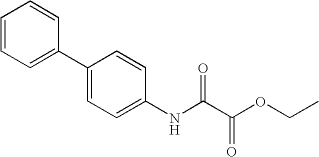

Chloro-oxo-acetic acid ethyl ester (847 mg, 6.2 mmol) was added a cold (0° C.) mixture of Biphenyl-4-ylamine (1.0 g, 5.9 mmol)(Aldrich, St. Louis, Mo.), DIPEA (2.3 mg, 3.9 mL, 17.7 mmol) in DCM (10 mL) and stirring was continued at ambient temperature for 1 hr. The reaction mixture was diluted with water, extracted with ethyl acetate, washed the organic layer with brine solution, dried over Na₂SO₄, and concentrated under reduced pressure to afford the residue. The residue obtained was purified by column chromatography (using 60-120 silica gel and 7% EtOAc in Hexane as eluent) to afford 1.2 g (75.4%) N-Biphenyl-4-yl-oxalamic acid ethyl ester.

### Step 2

### Synthesis of N-Biphenyl-4-yl-oxalamic acid

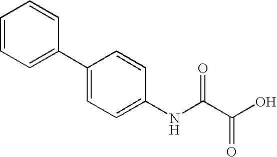

NaOH (891 mg, 22.27 mmol) in water (50 mL) was added to a stirred cold (0° C.) solution of N-Biphenyl-4-yl-oxalamic acid ethyl ester (1.2 mg, 4.4 mmol) in THF (50 mL) and the resulting mixture was stirred at ambient temperature for 1 hr. The reaction mixture was bubbled with N₂ gas to remove THF to afford the residue. The pH of the residue was adjusted to ‘5’ using 2N HCl. Filtered the solid precipitated to afford 1.0 g (93.4%) of N-Biphenyl-4-yl-oxalamic acid.

### Step 3

### Synthesis of (Biphenyl-4-ylamino)-oxo-acetyl chloride

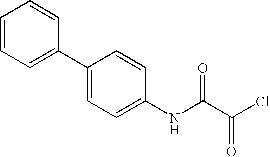

The preparation follows similar method as described in example 60, Step-3 to afford 107 mg of (Biphenyl-4-ylamino)-oxo-acetyl chloride.

### Step 4

### Synthesis of N-Biphenyl-4-yl-2-[4-(2-bromo-benzoyl)-piperazin-1-yl]-2-oxo-acetamide

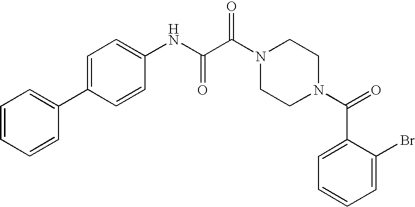

(Biphenyl-4-ylamino)-oxo-acetyl chloride (107 mg, 0.41 mmol) in DCM (2.5 mL) was added to cold (0° C.) solution of (2-Bromo-phenyl)-piperazin-1-yl-methanone hydrochloride (125 mg, 0.41 mmol) (prepared by the general method in earlier examples using 2-bromo benzoic acid as starting material, Aldrich, St. Louis, Mo.) and DIPEA (159 mg, 0.2 mL, 1.23 mmol) in DCM (2.5 mL) and stirring was continued at ambient temperature 1 hr. The reaction mixture was diluted with DCM, washed with water by brine solution. The organic layer thus collected was dried over sodium sulphate and concentrated under reduced pressure to afford the residue. The residue obtained was purified by column chromatography (using 60-120 silica gel and 15% EtOAc in Hexane as eluent) to afford 100 mg (49.3%) of N-Biphenyl-4-yl-2-[4-(2-bromo-benzoyl)-piperazin-1-yl]-2-oxo-acetamide. LCMS Purity: 96.22%. ¹H NMR (DMSO-d₆): δ 10.9 (d, 1H), 7.8-7.6 (m, 7H), 7.5-7.3 (m, 6H), 3.8-3.5 (m, 6H), 3.25 (t, 2H).

### Example 64

### N-Biphenyl-4-yl-2-oxo-2-[4-(3,4,5-trifluoro-benzoyl)-piperazin-1-yl]-acetamide

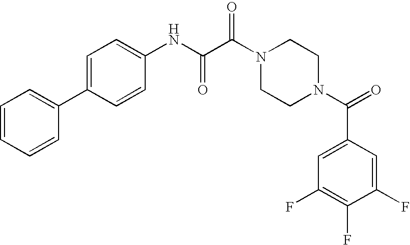

(Biphenyl-4-ylamino)-oxo-acetyl chloride (107 mg, 0.41 mmol) in DCM (5 mL) was added to cold (0° C.) solution of piperazin-1-yl-(3,4,5-trifluoro-phenyl)-methanone hydrochloride (115 mg, 0.41 mmol) prepared by the general method in earlier examples using 3,4,5-trifluoro benzoic acid as starting material, Aldrich, St. Louis, Mo.) and DIPEA (159 mg, 0.2 mL, 1.23 mmol) in DCM (5 mL) and stirring was continued at ambient temperature 1 hr. The reaction mixture was diluted with DCM, washed with water by brine solution. The organic layer thus collected was dried over sodium sulphate and concentrated under reduced pressure to afford the residue. The residue obtained was purified by column chromatography (using 60-120 silica gel and 18% EtOAc in Hexane as eluent) to afford 100 mg (49.3%) of N-Biphenyl-4-yl-2-oxo-2-[4-(3,4,5-trifluoro-benzoyl)-piperazin-1-yl]acetamide. LCMS Purity: 90.55%. ¹H NMR (DMSO-d₆): δ 11.0 (s, 1H), 7.0 (m, 2H), 7.7 (m, 4H), 7.6-7.3 (m, 4H), 7.4 (m, 1H), 3.7-3.33 (b, 8H).

### Enzyme Activity

Rat liver microsomes that have been induced to overexpress SCD-1 have been used as a source of SCD-1 activity. Tritiated stearoyl CoA labeled at position 9 and 10 is used as the substrate in the in vitro assay. SCD-1 activity of a compound is measured by detecting tritiated water released upon desaturation of the substrate. Desaturation by SCD-1 (a delta 9 desaturase) results in release of tritiated water which is measured in a scintillation counter. The compounds of the present invention typically show greater than 50% inhibition of SCD1 enzyme at 10 μM concentration.

The entire disclosures of all applications, patents and publications cited herein are hereby incorporated by reference.

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

What is claimed is:

1. A compound with chemical structure: 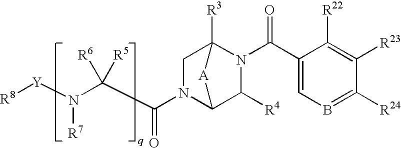
wherein
R²² is hydrogen, halogenated alkyl or halogen;
B is N or CR²⁵ where R²⁵ is hydrogen or halogen;
Y is —C(O)— or —CH₂—;
A is absent or is —CH₂—;
R³ and R⁴ are each, independently, hydrogen or alkyl;
R⁵ to R⁷ are each, independently, hydrogen or alkyl;
R⁸ is aryl or heteroaryl;
q is 0 or 1;
provided, however, that when q is 0, then y is —C(O)—, A is absent, and B is CR²⁵;
provided, however, that when R²² is hydrogen or halogen, then B is CR²⁵, and R²⁵ is halogen;
provided, however, that when R²² is hydrogen, then R²³ to R²⁵ are each independently halogen, and B is CR²⁵;
provided, however, that when R²² is halogenated alkyl, then R²³ to R²⁴ are both hydrogen and B is CR²⁵ or N;
wherein, when present, any aryl, heteroaryl, or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkylhio, arylthio, alkylsulfinyl, alkylsulfonyl, arylsulfinyl, arylsulfonyl, heteroarylsulfinyl, heteroarylsulfonyl alkoxycarbonyl, aryloxycarbonyl, heteroaryloxycarbonyl, and combinations thereof;
and pharmaceutically acceptable salts or solvates or N-oxides thereof, or solvates of pharmaceutically acceptable salts thereof, or pharmaceutically acceptable salts or solvates of N-oxides thereof; or prodrugs thereof.

2. A compound according to claim 1, selected from the group consisting of
N-{2-Oxo-2-[4-(2-trifluoromethyl-benzoyl)-piperazin-1-yl]-ethyl}-4-pyridin-3-yl-benzamide,
5-(2-Hydroxy-phenyl)-isoxazole-3-carboxylic acid {2-[4-(5-fluoro-2-trifluoromethyl-benzoyl)-piperazin-1-yl]-2-oxo-ethyl}-amide,
5-(2-Amino-phenyl)-isoxazole-3-carboxylic acid {2-oxo-2-[4-(2-trifluoromethyl-benzoyl)-piperazin-1-yl]-ethyl}-amide,
3-[1,3,4]Oxadiazol-2-yl-N-{2-oxo-2-[4-(2-trifluoromethyl-benzoyl)-piperazin-1-yl]-ethyl}-benzamide,
Naphthalene-2-carboxylic acid {2-[4-(5-fluoro-2-trifluoromethyl-benzoyl)-piperazin-1-yl]-2-oxo-ethyl}-amide,
5-Phenyl-1H-pyrazole-3-carboxylic acid {2-[3,5-dimethyl-4-(2-trifluoromethyl-benzoyl)-piperazin-1-yl]-2-oxo-ethyl}-amide,
1-Phenyl-1H-[1,2,3]triazole-4-carboxylic acid {2-oxo-2-[4-(4-trifluoromethyl-pyridine-3-carbonyl)-piperazin-1-yl]-ethyl}-amide,
5-Phenyl-1H-pyrazole-3-carboxylic acid {2-[5-(5-fluoro-2-trifluoromethyl-benzoyl)-2,5-diaza-bicyclo[2.2.1]hept-2-yl]-2-oxo-ethyl}-amide,
N-{2-[4-(5-Fluoro-2-trifluoromethyl-benzoyl)-piperazin-1-yl]-2-oxo-ethyl}-4-(1H-tetrazol-5-yl)-benzamide,
1-(2-Fluoro-phenyl)-1H-[1,2,3]triazole-4-carboxylic acid {2-[4-(5-fluoro-2-trifluoromethyl-benzoyl)-piperazin-1-yl]-2-oxo-ethyl}-amide,
1-(5-Methyl-isoxazol-3-yl)-1H-[1,2,3]triazole-4-carboxylic acid {2-[4-(5-fluoro-2-trifluoromethyl-benzoyl)-piperazin-1-yl]-2-oxo-ethyl}-amide,
1-[4-(5-Fluoro-2-trifluoromethyl-benzoyl)-piperazin-1-yl]-2-[(1-phenyl-1H-[1,2,3]triazol-4-ylmethyl)-amino]-ethanone,
1-(2-Hydroxy-phenyl)-1H-[1,2,3]triazole-4-carboxylic acid {2-[4-(5-fluoro-2-trifluoromethyl-benzoyl)-piperazin-1-yl]-2-oxo-ethyl}-amide,
5-(3-Fluoro-phenyl)-1H-pyrazole-3-carboxylic acid {2-[4-(5-fluoro-2-trifluoromethyl-benzoyl)-piperazin-1-yl]-2-oxo-ethyl}-amide,
5-(4-Fluoro-phenyl)-1H-pyrazole-3-carboxylic acid {2-[4-(5-fluoro-2-trifluoromethyl-benzoyl)-piperazin-1-yl]-2-oxo-ethyl}-amide,
4-Phenyl-pyrazole-1-carboxylic acid {2-oxo-2-[4-(4-trifluoromethyl-pyridine-3-carbonyl)-piperazin-1-yl]-ethyl}-amide,
5-(4-Fluoro-phenyl)-isoxazole-3-carboxylic acid {2-[4-(5-fluoro-2-trifluoromethyl-benzoyl)-piperazin-1-yl]-2-oxo-ethyl}-amide,
N-{2-[4-(5-Fluoro-2-trifluoromethyl-benzoyl)-piperazin-1-yl]-2-oxo-ethyl}-6-pyrrolidin-1-yl-nicotinamide,
1-Pyridin-3-yl-1H-[1,2,3]triazole-4-carboxylic acid {2-[4-(5-fluoro-2-trifluoromethyl-benzoyl)-piperazin-1-yl]-2-oxo-ethyl}-amide,
Imidazo[1,2-a]pyridine-2-carboxylic acid {2-[4-(5-fluoro-2-trifluoromethyl-benzoyl)-piperazin-1-yl]-2-oxo-ethyl}-amide,
5-(3-Hydroxy-phenyl)-1H-pyrazole-3-carboxylic acid {2-[4-(5-fluoro-2-trifluoromethyl-benzoyl)-piperazin-1-yl]-2-oxo-ethyl}-amide,
5-(4-Hydroxy-phenyl)-1H-pyrazole-3-carboxylic acid {2-[4-(5-fluoro-2-trifluoromethyl-benzoyl)-piperazin-1-yl]-2-oxo-ethyl}-amide, and
5-Pyridin-3-yl-1H-pyrazole-3-carboxylic acid {2-[4-(5-fluoro-2-trifluoromethyl-benzoyl)-piperazin-1-yl]-2-oxo-ethyl}-amide,
1-[4-(5-Fluoro-2-trifluoromethyl-benzoyl)-piperazin-1-yl]-2-[(1-phenyl-1H-[1,2,3]triazol-4-ylmethyl)-amino]-ethanone hydrochloride,
5-(2-Hydroxy-phenyl)-1H-pyrazole-3-carboxylic acid {2-oxo-2-[4-(3,4,5-trifluoro-benzoyl)-piperazin-1-yl]-ethyl}-amide,
2-[(Biphenyl-4-ylmethyl)-amino]-1-[4-(3,4,5-trifluoro-benzoyl)-piperazin-1-yl]-ethanone,
1-Pyridin-3-yl-1H-[1,2,3]triazole-4-carboxylic acid {2-oxo-2-[4-(3,4,5-trifluoro-benzoyl)-piperazin-1-yl]-ethyl}-amide,
1-Pyridin-2-yl-1H-[1,2,3]triazole-4-carboxylic acid {2-oxo-2-[4-(3,4,5-trifluoro-benzoyl)-piperazin-1-yl]-ethyl}-amide,
1-Cyclopentyl-1H-[1,2,3]triazole-4-carboxylic acid {2-oxo-2-[4-(3,4,5-trifluoro-benzoyl)-piperazin-1-yl]-ethyl}-amide,
2-[(Biphenyl-4-ylmethyl)-amino]-1-[4-(3,4,5-trifluoro-benzoyl)-piperazin-1-yl]-ethanone hydrochloride.
1-Pyridin-3-yl-1H-[1,2,3]triazole-4-carboxylic acid {2-[4-(2-chloro-5-fluoro-benzoyl)-piperazin-1-yl]-2-oxo-ethyl}-amide,
1-Pyridin-2-yl-1H-[1,2,3]triazole-4-carboxylic acid {2-[4-(2-chloro-5-fluoro-benzoyl)-piperazin-1-yl]-2-oxo-ethyl}-amide,
1-Phenyl-1H-imidazole-4-carboxylic acid {2-[4-(2-chloro-5-fluoro-benzoyl)-piperazin-1-yl]-2-oxo-ethyl}-amide,
1-Cyclopropyl-1H-[1,2,3]triazole-4-carboxylic acid {2-[4-(2-chloro-5-fluoro-benzoyl)-piperazin-1-yl]-2-oxo-ethyl}-amide,
1-Morpholin-4-yl-1H-[1,2,3]triazole-4-carboxylic acid {2-[4-(2-chloro-5-fluoro-benzoyl)-piperazin-1-yl]-2-oxo-ethyl}-amide,
N-{2-[4-(2-Chloro-5-fluoro-benzoyl)-piperazin-1-yl]-2-oxo-ethyl}-4-(2-oxo-pyrrolidin-1-yl)-benzamide,
1-Cyclopentyl-1H-[1,2,3]triazole-4-carboxylic acid {2-[4-(2-chloro-5-fluoro-benzoyl)-piperazin-1-yl]-2-oxo-ethyl}-amide,
9H-Carbazole-3-carboxylic acid {2-[4-(5-fluoro-2-trifluoromethyl-benzoyl)-piperazin-1-yl]-2-oxo-ethyl}-amide,
5-(3-Hydroxy-phenyl)-isoxazole-3-carboxylic acid {2-[4-(5-fluoro-2-trifluoromethyl-benzoyl)-piperazin-1-yl]-2-oxo-ethyl}-amide,
5-(4-Hydroxy-phenyl)-isoxazole-3-carboxylic acid {2-[4-(5-fluoro-2-trifluoromethyl-benzoyl)-piperazin-1-yl]-2-oxo-ethyl}-amide,
5-(3-Fluoro-phenyl)-isoxazole-3-carboxylic acid {2-[4-(5-fluoro-2-trifluoromethyl-benzoyl)-piperazin-1-yl]-2-oxo-ethyl}-amide,
5-Pyridin-2-yl-1H-pyrazole-3-carboxylic acid {2-[4-(5-fluoro-2-trifluoromethyl-benzoyl)-piperazin-1-yl]-2-oxo-ethyl}-amide,
1-(3-Fluoro-phenyl)-1H-[1,2,3]triazole-4-carboxylic acid {2-[4-(5-fluoro-2-trifluoromethyl-benzoyl)-piperazin-1-yl]-2-oxo-ethyl}-amide,
1-(3-Fluoro-phenyl)-1H-[1,2,3]triazole-4-carboxylic acid {2-oxo-2-[4-(3,4,5-trifluoro-benzoyl)-piperazin-1-yl]-ethyl}-amide,
1-(3-Fluoro-phenyl)-1H-[1,2,3]triazole-4-carboxylic acid {2-[4-(2-chloro-5-fluoro-benzoyl)-piperazin-1-yl]-2-oxo-ethyl}-amide,
1-(2-Fluoro-phenyl)-1H-[1,2,3]triazole-4-carboxylic acid {2-[4-(2-chloro-5-fluoro-benzoyl)-piperazin-1-yl]-2-oxo-ethyl}-amide,
1-(2-Fluoro-phenyl)-1H-[1,2,3]triazole-4-carboxylic acid {2-[4-(2-chloro-5-fluoro-benzoyl)-piperazin-1-yl]-2-oxo-ethyl}-amide,
4-(2-Fluoro-phenyl)-pyrazole-1-carboxylic acid {2-oxo-2-[4-(4-trifluoromethyl-pyridine-3-carbonyl)-piperazin-1-yl]-ethyl}-amide,
1-m-Tolyl-1H-[1,2,3]triazole-4-carboxylic acid {2-[4-(5-fluoro-2-trifluoromethyl-benzoyl)-piperazin-1-yl]-2-oxo-ethyl}-amide,
1-m-Tolyl-1H-[1,2,3]triazole-4-carboxylic acid {2-[4-(2-chloro-5-fluoro-benzoyl)-piperazin-1-yl]-2-oxo-ethyl}-amide,
1-o-Tolyl-1H-[1,2,3]triazole-4-carboxylic acid {2-[4-(5-fluoro-2-trifluoromethyl-benzoyl)-piperazin-1-yl]-2-oxo-ethyl}-amide,
1-m-Tolyl-1H-[1,2,3]triazole-4-carboxylic acid {2-oxo-2-[4-(3,4,5-trifluoro-benzoyl)-piperazin-1-yl]-ethyl}-amide,
1-(2-Cyano-phenyl)-1H-[1,2,3]triazole-4-carboxylic acid {2-[4-(2-chloro-5-fluoro-benzoyl)-piperazin-1-yl]-2-oxo-ethyl}-amide,
5-(4-Fluoro-phenyl)-isoxazole-3-carboxylic acid {2-[4-(2-chloro-5-fluoro-benzoyl)-piperazin-1-yl]-2-oxo-ethyl}-amide,
6-Pyrazol-1-yl-imidazo[1,2-a]pyridine-2-carboxylic acid {2-[4-(2-chloro-5-fluoro-benzoyl)-piperazin-1-yl]-2-oxo-ethyl}-amide,
4-(5-Fluoro-2-trifluoromethyl-benzoyl)-1-{2-[(5-phenyl-1H-pyrazole-3-carbonyl)-amino]-acetyl}-piperazine-2-carboxylic acid,
1-(5-Fluoro-2-trifluoromethyl-benzoyl)-4-{2-[(5-phenyl-1H-pyrazole-3-carbonyl)-amino]-acetyl}-piperazine-2-carboxylic acid,
and pharmaceutically acceptable salts thereof, pharmaceutically acceptable solvates thereof, and solvates of pharmaceutically acceptable salts thereof.

3. A compound chosen from:
2-{2-Oxo-2-[4-(2-trifluoromethyl-benzoyl)-piperazin-1-yl]-acetylamino}-benzoic acid methyl ester,
2-Oxo-N-(6-phenyl-pyridin-3-yl)-2-[4-(2-trifluoromethyl-benzoyl)-piperazin-1-yl]-acetamide,
N-Biphenyl-4-yl-2-[4-(5-fluoro-2-trifluoromethyl-benzoyl)-piperazin-1-yl]-2-oxo-acetamide,
[4-(5-Fluoro-2-trifluoromethyl-benzoyl)-piperazin-1-yl]-oxo-acetic acid ethyl ester,
3-{2-[4-(5-Fluoro-2-trifluoromethyl-benzoyl)-piperazin-1-yl]-2-oxo-acetylamino}-N-methyl-benzamide,
2-[4-(5-Fluoro-2-trifluoromethyl-benzoyl)-piperazin-1-yl]-2-oxo-N-(4-phenoxy-phenyl)-acetamide,
N-Biphenyl-4-yl-2-[4-(2-bromo-benzoyl)-piperazin-1-yl]-2-oxo-acetamide, and
N-Biphenyl-4-yl-2-oxo-2-[4-(3,4,5-trifluoro-benzoyl)-piperazin-1-yl]-acetamide,
and pharmaceutically acceptable salts thereof, pharmaceutically acceptable solvates thereof, and solvates of pharmaceutically acceptable salts thereof.

4. A pharmaceutical composition comprising a compound according to claim 1 and a pharmaceutically acceptable carrier.

5. A method for treating a condition that responds to a stearoyl-CoA desaturase inhibitor comprising administering to a patient in need thereof an effective amount of a composition according to claim 4.

6. The method according to claim 5, wherein the condition is obesity or diabetes.
